(19) United States
(12) Patent Application Publication
(10) Pub. No.: US 2025/0157574 A1
(43) Pub. Date: May 15, 2025

(54) BYPASSING SANGER CONFIRMATION FOR SMALL VARIANTS IN GENETIC DISORDER CLINICAL TESTING

(71) Applicant: Laboratory Corporation of America Holdings, Burlington, NC (US)

(72) Inventors: Muqing Yan, Austin, TX (US); Jennifer Elizabeth Reiner, Framingham, MA (US); Qiandong Zeng, Belmont, MA (US); Stanley Ian Letovsky, Milton, MA (US); Angela Antoinette Kenyon, Worcester, MA (US); Natalia Tszine Leach, Burlington, NC (US)

(21) Appl. No.: 18/942,335

(22) Filed: Nov. 8, 2024

Related U.S. Application Data
(60) Provisional application No. 63/597,231, filed on Nov. 8, 2023.

(51) Int. Cl. G16B 20/20; G16B 40/20

(52) U.S. Cl. CPC G16B 20/20; G16B 40/20

(57) ABSTRACT

The present disclosure relates to a sequencing platform and workflow that leverages machine learning algorithms in genetic assays to bypass confirmatory Sange sequencing for high-confidence variants. Aspects are directed towards performing next generation sequencing (NGS) on nucleic acid obtained from a biological sample of a subject to generate sequencing data; extracting variant information from the sequencing data, wherein the information includes variant types and quality features; clustering variants into a subset of variants based on the variant types; generating a predicted status of each variant in the subset of variants based on the one or more quality features using a first machine learning model; generating a confirmatory status of each variant with an unknown status as the predicted status using a second machine learning model; and performing Sanger sequencing on nucleic acid molecules comprising variants with the absence status.

### CROSS-REFERENCES TO RELATED APPLICATIONS

The present application claims priority and benefit from U.S. Provisional Application No. 63/597,231, filed Nov. 8, 2023, the entire contents of which are incorporated herein by reference for all purposes.

### FIELD

The present disclosure relates to a clinical laboratory Next Generation Sequencing (NGS) assay platform, and in particular, to techniques that leverage machine learning algorithms in diagnostic and screening assays to bypass confirmatory testing for high-confidence variants.

### BACKGROUND

Genetic screening is a process that involves examining the DNA of an individual in the search for changes or mutations that may be associated with illness or disease. Types of mutations detected in these screens can include single base alterations (e.g., single nucleotide variants (SNVs)) and small and large structural alterations (e.g., insertion-deletions (indels), copy number variants (CNVs), and chromosomal rearrangements/translocations). Clinical laboratories perform a number of different genetic screenings related to wellness (e.g., predictive/predispositional testing for cancers) and fertility/pregnancy (e.g., newborn screening, carrier screening, prenatal diagnostic testing) as part of routine care for their patients. Samples for testing are frequently collected in the form of blood, making the collection process relatively easy, safe, and noninvasive. Historically, genetic screening was conducted on a catalog of known disease-causing variants, but contemporary practices now typically involve full gene sequencing of anywhere from 1 to hundreds of genes associated with any number of health conditions.

For genetic screening to have robust performance and provide accurate results, sufficient sequencing coverage of the targeted regions must be achieved. The predominant sequencing techniques used in clinical laboratories include Sanger sequencing and Next Generation Sequencing methods. Sanger sequencing is a first-generation DNA sequencing method that has long been considered the gold standard for the accurate detection of small sequence variants. First generation sequencing techniques, like Sanger, utilize a chain-termination method wherein specialized fluorescently labelled DNA bases (dideoxynucleotides or ddNTPs) are randomly incorporated into growing DNA chains of nucleotides (A, C, G, T) generating different length DNA fragments. Fragments are size separated by capillary electrophoresis and a laser is used to excite the unique fluorescence signal associated with each ddNTP. As the fluorescence signal is recorded, a chromatograph is generated, showing which base is present at a given location of the target region being sequenced. In the clinical setting, Sanger provides flexibility for testing single or small batch samples for prenatal or carrier testing and can provide results in a relatively short period of time. However, Sanger sequencing is labor intensive and not amenable to high-throughput sequencing of large panels.

Next Generation Sequencing (NGS) has largely replaced Sanger sequencing due to its massively parallel sequencing capabilities, allowing for millions of bases to be concurrently sequenced instead of just a few hundred by Sanger. Briefly, NGS uses a process known as clonal amplification to amplify the DNA fragments of a patient sample and bind them to a flow cell. Then, a sequencing by synthesis method is used where fluorescently labeled nucleotides compete for addition onto a growing chain based on the sequence of the template. A light source is used to excite the unique fluorescence signal associated with each nucleotide and the emission wavelength and fluorescence signal intensity determine the base call. Each lane in a flow cell can hold hundreds to millions of DNA templates, giving NGS its massively parallel sequencing capabilities. Importantly, NGS technologies have greatly improved the flexibility of genetic screenings, providing highly sensitive and accurate high-throughput platforms for large-scale genomic testing, including sequencing of entire genomes and exomes.

Targeted genomic sequencing (TGS), whole genome sequencing (WGS), and whole exome sequencing (WES) are three sequencing approaches used in the analysis of genetic material, each with its own unique applications and benefits. TGS focuses on a panel of genes or targets known to contain DNA alterations with strong associations to the pathogenesis of disease and/or clinical relevance. DNA alterations typically include single nucleotide variants (SNVs), deletions and/or insertions (indels), inversions, translocations/fusions, and copy number variations (CNV). Because only specific regions of interests from the genome are interrogated in TGS, a much greater sequencing depth is achieved (number of times a given nucleotide is sequenced), and highly accurate variant calls are obtained at a significantly reduced cost and data burden compared to more global NGS methods such as WGS and WES. Moreover, TGS can identify low frequency variants in targeted regions with high confidence and is thus suitable for profiling low-quality and fragmented clinical DNA samples (e.g., as seen in cell-free DNA). This approach is often employed in clinical settings where specific genetic markers are being investigated, such as in the diagnosis of certain cancers or inherited genetic disorders.

WGS, on the other hand, involves sequencing the entire genome, providing a comprehensive overview of all genetic material, including coding and non-coding regions (e.g., covering all or substantially all the 3 billion DNA base pairs that make up an entire human genome). WGS offers an unbiased approach to genetic analysis, capturing a wide array of genetic variations, including single nucleotide variants, insertions, deletions, copy number variations, and structural variants. This method is invaluable for research and clinical diagnostics when a holistic view of the genome is required, for instance, in complex diseases with multifactorial genetic contributions such as cancer diagnostics.

Whole exome sequencing (WES) falls somewhere in between TGS and WGS. WES focuses exclusively on the exonic regions of the genome, which constitute about 1-2% of the genome but harbor approximately 85% of known disease-causing mutations. Exons are defined as the sequences in a gene that encode proteins as well as the upstream and downstream untranslated regions (UTRs) that mediate transcript stability, localization, and translation. Approximately 2% of the human genome is comprised of exons. Because the exome is so much smaller than the genome, exomes can be sequenced at a much greater depth (number of times a given nucleotide is sequenced) for lower cost. This greater depth of coverage improves calling accuracy and reduces the likelihood of missing deleterious variants. Exome sequencing also provides an advantage to clinical laboratories that use computational tools to create in silico panels from an exome library as updates to the panel can be made without redesigning and revalidating an assay. That is, WES provides a more cost-effective solution than WGS while still covering a significant portion of clinically relevant genetic information, making it a popular choice for diagnosing certain diseases (e.g., Mendelian disorders) and uncovering novel genetic mutations linked to diseases.

### BRIEF SUMMARY

In various embodiments, a computer-implemented method is provided that comprises: inputting an annotated file for a one or more variants into an assay pipeline, where the annotated file was generated as part of performing a whole exome sequencing assay, the one or more variants comprise alterations to a DNA sequence not found in a reference sequence and the alterations can be heterozygous single nucleotide variants, homozygous single nucleotide variants, heterozygous insertion-deletions, or homozygous insertion-deletions, the assay pipeline comprises a first tier and a second tier, the first tier comprises at least two machine learning models, and the second tier comprises a third machine learning model; classifying the one or more variants based on one or more nucleotides or chromosomal regions affected, wherein the one or more variants are a heterozygous single nucleotide variants; determining if the heterozygous single nucleotide variants, using the first-tier machine learning models, is absent, present, or unknown; bypassing Sanger sequencing confirmation when the one or more heterozygous single nucleotide variants are classified as present and meet criteria and quality thresholds of the first-tier machine learning models, or confirming that Sanger sequencing is required when the one or more heterozygous single nucleotide variants are classified as present and do not meet the criteria and the quality thresholds of the first-tier machine learning models; and generating a report that identifies which variants require Sanger sequencing confirmation.

In some embodiments, the annotated file comprises quality features characteristic of the whole exome sequencing assay.

In some embodiments, the quality features are selected from a list comprising read count, read coverage, frequency, forward count, reverse count, forward/reverse ratio, average quality, probability, read position probability, read direction probability, homopolymer, homopolymer length, and complex region.

In some embodiments, the at least two machine learning models comprise a logistic regression model and a random forest classifier; and the second tier comprising the third machine learning model comprises a gradient boosting model.

In some embodiments, the criteria of the first-tier machine learning models include a probability threshold of a logistic regression model and a probability threshold of a random forest classifier.

In some embodiments, the quality thresholds of the first-tier machine learning models refer to allele frequency and read coverage.

In some embodiments, when the heterozygous single nucleotide variants are classified as absent, Sanger sequencing confirmation is required.

In some embodiments, when the heterozygous single nucleotide variants are classified as unknown, a determination is made as to whether the heterozygous single nucleotide variants (i) meet the quality thresholds of the first-tier machine learning models and are input into the second-tier machine learning model, or (ii) do not meet quality thresholds of the first-tier machine learning models and Sanger sequencing confirmation is required.

In some embodiments, the computer-implemented method further comprises: determining whether the unknown heterozygous single nucleotide variants, using the second-tier machine learning model, is absent or present, wherein when the unknown heterozygous single nucleotide variants are classified as absent, Sanger sequencing confirmation is required, and when the unknown heterozygous single nucleotide variants are classified as present, Sanger sequencing confirmation is bypassed.

In some embodiments, the computer-implemented method further comprises: when the heterozygous single nucleotide variants are classified as unknown, determining the heterozygous single nucleotide variants do not meet the quality thresholds of the at least two first-tier machine learning models; and performing Sanger sequencing confirmation on the unknown heterozygous single nucleotide variants.

In some embodiments, the homozygous single nucleotide variants require Sanger sequencing confirmation.

In some embodiments, the homozygous insertion-deletion variants require Sanger sequencing confirmation; and the heterozygous insertion-deletion variants either (i) pass exemption criteria and are bypassed for Sanger sequencing confirmation, or (ii) do not pass the exemption criteria and Sanger sequencing confirmation is required.

In some embodiments, the exemption criteria include the heterozygous insertion-deletion variants being on a predetermined exemption list and meeting the quality thresholds of the first-tier machine learning models.

In some embodiments, the computer-implemented method further comprising when Sanger confirmation is required, executing Sanger sequencing on the one or more variants that fail to meet the criteria and quality thresholds of the first-tier machine learning models and display quality features significantly associated with false positive variants, and when Sanger sequencing is not required, bypassing Sanger sequencing confirmation on the one or more variants that do meet the criteria and quality thresholds of the first-tier machine learning models and display quality features significantly associated with true positive variants.

In various embodiments, a computer-implemented method is provided that comprises: training a one or more machine learning models to predict whether one or more variants are true positives or false positives, wherein training comprises: accessing high-confidence variant data that are labeled as truths; accessing annotated files that comprise the one or more variants and their quality features, wherein the annotated files were generated as part of performing a whole exome sequencing assay; generating a labeled variant dataset by annotating the one or more variants with truth labels based on the high-confidence variant data; splitting the labeled variant dataset, using stratification of the truth labels, to generate a first subset of training data and a first subset of testing data; executing a first training and testing phase, using the first subset of training data, wherein the first training and testing phase comprises performing a leave-one-out cross-validation (LOOCV) method to evaluate a false positive capture rate and a true positive flagging rate for the one or more machine learning models across different genomic backgrounds; executing a second training and testing phase, using the first subset of training data and the first subset of testing data, wherein the second training and testing phase comprises performing a one or more rounds of classical training to generate a one or more final machine learning models to be used in a first-tier and a second tier of a pipeline; selecting at least two of the one or more final machine learning models for the first-tier and one of the one or more final machine learning models for the second tier to generate the pipeline; and executing a final validation phase, using the labeled variant dataset, on the pipeline to validate the first-tier and second-tier final machine learning models; and providing the validated first-tier and second-tier machine learning models.

In some embodiments, the one or more machine learning models comprises a logistic regression model, a random forest model, an EasyEnsemble model, an AdaBoost model, or a gradient boosting model.

In some embodiments, the quality features comprise: read count, read coverage, frequency, forward count, reverse count, forward/reverse ratio, average quality, probability, read position probability, read direction probability, homopolymer, homopolymer length, and complex region.

In some embodiments, the labeled variant dataset comprises variants with a true positive label and a false positive label, wherein the true positive label refers to variants found in both the high-confidence variant data and the annotated files, and the false positive label refers to variants absent in the high-confidence variant data but present in the annotated files.

In some embodiments, the false positive capture rate refers to the sensitivity of the one or more machine learning models to capture false positive variants and the true positive flagging rate refers to the false positive rate of one or more machine learning models to tag a true positive variant as a false positive variant.

In some embodiments, the leave-one-out cross-validation (LOOCV) method comprises iterative operations of training the one or more machine learning models on one less of the total number of samples, testing one or more partially trained machine learning models on the left-out sample, repeating the LOOCV method based on the total number of samples so that each sample is left out once, calculating the false positive capture rate and the true positive flagging rate, and generating one or more cross-validated machine learning models.

In some embodiments, the LOOCV method further comprises: splitting the first subset of training data into a cross-validation training dataset and a cross-validation testing dataset, wherein: the cross-validation training dataset comprise one less of the total number of samples from the first subset of training data and is used in the LOOCV method, and the cross-validation testing dataset comprises the left-out sample and is used in the LOOCV method; and evaluating, using all the quality features, the false positive capture rate and true positive flagging rate of a one or more cross-validated machine learning models across different genetic backgrounds.

In some embodiments, a first of the one or more rounds of classical training comprises: scaling the quality features comprising the first subset of training data to generate a scaled subset of training data; training, the one or more cross-validated machine learning models, on the scaled subset of training data to generate one or more post-trained machine learning models, wherein training comprises fine tuning a set of parameters for the one or more cross-validated machine learning models that maximizes the false positive capture rate and minimizes the true positive flagging rate so that a value of the loss or error function using the set of parameters is smaller than a value of the loss or error function using another set of parameters in a previous iteration; evaluating, for the one or more post-trained machine learning models, coefficient values or importance values of the quality features to identify high-impact quality features and to select the one or more post-trained machine learning models that did not show an improvement in their coefficient values or importance values; repeating the training, using the first subset of training data without scaling, for the selected one or more post-trained machine learning models that did not show an improvement in their coefficient values or importance values; generating one or more post-trained machine learning models trained on all the quality features; testing, using the first subset of testing data, the one or more post-trained machine learning models trained on the high-impact quality features and the one or more post-trained machine learning models trained on all the quality features to validate that training on the high-impact quality features or all the quality features improves the false positive capture rate and the true positive flagging rate of the one or more optimized machine learning models and to generate one or more improved machine learning models; and generating the one or more improved machine learning models trained on either: (i) the high-impact quality features or (ii) all the quality features.

In some embodiments, the one or more high-impact quality features are selected from the quality features.

In some embodiments, the coefficient values or the importance values for the quality features reflect the relative contribution of each quality feature to the associated true positive or false positive variant label.

In some embodiments, a second of the one or more rounds of the classical training method comprises: a second of the one or more rounds of the classical training method comprises: oversampling, the false positive variants comprising the first subset of training data to generate a balanced dataset; training, the one or more improved machine learning models trained on the high-impact quality features and the one or more improved machine learning models trained on all the quality features, on the balanced dataset to generate one or more optimized machine learning models, wherein training comprises fine tuning a set of parameters for the one or more improved machine learning models trained on the high-impact quality features and the one or more improved machine learning models trained on all the quality features that maximizes the false positive capture rate and minimizes the true positive flagging rate so that a value of the loss or error function using the set of parameters is smaller than a value of the loss or error function using another set of parameters in a previous iteration; evaluating, for the one or more optimized machine learning models trained on the high-impact quality features and balanced data and the one or more optimized machine learning models trained on all the quality features and balanced data, the false positive capture rate and the true positive flagging rate for each of the one or more optimized machine learning models trained on the balanced dataset to select the one or more optimized machine learning models that did not show an improvement in the false positive capture rate and the true positive flagging rate; repeating the training, using the first subset of training data without oversampling, for the selected one or more optimized machine learning models trained on the high-impact quality features and the one or more optimized machine learning models trained on all the quality features that did not show improvement in their false positive capture rate and true positive flagging rate after training on balanced data generating one or more optimized machine learning models trained on the high-impact quality features and imbalance data and one or more optimized machine learning models trained on all the quality features and imbalance data that show an improvement in the false positive capture rate and the true positive flagging rate, wherein the imbalanced data is the first subset of training data without oversampling; testing, using the first subset of testing data, the one or more optimized machine learning models trained on the high-impact quality features and balanced data, the one or more optimized machine learning models trained on all the quality features and balanced data, the one or more optimized machine learning models trained on the high-impact quality features and imbalance data, and the one or more optimized machine learning models trained on all the quality features and imbalance data to validate that training on the balanced dataset or the imbalanced dataset improves the false positive capture rate and the true positive flagging rate of the one or more optimized machine learning models and to generate one or more final machine learning models; and generating the one or more final machine learning models trained on either: (i) all the quality features and the imbalanced data, (ii) all the quality features and the balanced data, (iii) the high impact quality features and the imbalanced data, or (iv) the high impact quality features and the balanced data.

In some embodiments, the oversampling comprises either simple oversampling (SOS) or synthetic minority oversampling (SMOTE).

In some embodiments, selecting the at least two machine learning models from the set of final machine learning models for the first-tier comprises selecting the logistic regression model and the random forest classifier, and selecting one of the one or more final machine learning models for the second tier comprise selecting the gradient boosting model.

In some embodiments, the logistic regression model is trained on the high-impact quality features and SOS balanced data, the random forest classifier is trained all the quality features and the imbalanced data, and the gradient boosting model is trained on all the quality features and the imbalanced data.

In various embodiments, a computer-implemented method is provided that comprises: performing next generation sequencing (NGS) on nucleic acid obtained from a biological sample of a subject to generate sequencing data; extracting information of a set of variants from the sequencing data, wherein the information of the set of variants comprises a type of each variant in the set of variants and one or more quality features of each variant in the set of variants; clustering the set of variants into one or more subsets of variants based on the type of each variant in the set of variants; generating, using a first machine learning model, a predicted status of each variant in at least one subset of the one or more subsets of variants based on the one or more quality features, wherein the predicted status is a presence status, an absence status, or an unknown status; generating, using a second machine learning model, a confirmatory status of each variant with the unknown status as the predicted status, wherein the confirmatory status is a presence status or an absence status; and performing Sanger sequencing on nucleic acid molecules comprising variants with the absence status as the predicted status or the confirmatory status to confirm an existence of the variants.

In some embodiments, the computer-implemented method further comprises generating a testing report for the subject based on the sequencing data, the information of the set of variants, the predicted status of each variant in the at least one subset of the one or more subsets of variants, the confirmatory status of each variant with the unknown status as the predicted status, and/or results of the Sanger sequencing.

In some embodiments, the type of each variant is a heterozygous single nucleotide variant (SNV), a homozygous SNV, a heterozygous insertion-deletion (indel), or a homozygous indel.

In some embodiments, each variant in the at least one subset of the one or more subsets of variants is a heterozygous SNV.

In some embodiments, the computer-implemented method further comprises performing Sanger sequencing on regions corresponding to homozygous SNVs or homozygous indels.

In some embodiments, the NGS is whole exome sequencing or targeted sequencing.

In some embodiments, the computer-implemented method further comprises extracting information of a second set of variants from the sequencing data, wherein the second set of variants comprises variants in complexity regions; and performing Sanger sequencing on regions corresponding to the second set of variants.

In some embodiments, the computer-implemented method further comprises determining (i) an allele frequency and (ii) a read coverage for a variant with a present or unknown status as the predicted status; determining (i) the allele frequency or (ii) the read coverage failing a predetermined criterion; and performing Sanger sequencing on a region corresponding to the variant.

In some embodiments, the predetermined criterion comprises (i) an allele frequency of between about 36% and about 65% and (ii) an average read coverage of at least 30×.

In some embodiments, the one or more quality features comprise features selected from the group consisting of: read count, read coverage, frequency, forward count, reverse count, forward/reverse ratio, average quality, probability, read position probability, read direction probability, homopolymer, homopolymer length, and complex region.

In some embodiments, the computer-implemented method further comprises performing NGS on reference samples obtained from a database to generate reference sequencing data; and training the first machine learning model and the second machine learning model using labeled variant data obtained from the database and the reference sequencing data.

In some embodiments, the first machine learning model is a model combining logistic regression and random forest, and/or the second machine learning model is a gradient boosting model.

In some embodiments, a computer-program product is provided that is tangibly embodied in a non-transitory machine-readable medium and that includes instructions configured to cause one or more data processors to perform part or all of one or more methods disclosed herein.

In some embodiments, a system is provided that includes one or more data processors and a non-transitory computer readable medium containing instructions which, when executed on the one or more data processors, cause the one or more data processors to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the disclosure. Thus, it should be understood that although the present application has been specifically disclosed by embodiments and optional features, modification and variation of the concepts herein disclosed may be resorted to by those skilled in the art, and that such modifications and variations are considered to be within the scope of this application as defined by the appended claims.

### BRIEF DESCRIPTION OF THE DRAWINGS

The drawings illustrate certain embodiments of the technology and are not limiting. For clarity and ease of illustration, the drawings are not made to scale and, in some instances, various aspects may be shown exaggerated or enlarged to facilitate an understanding of particular embodiments.

FIG. 1 shows an exemplary computing environment for implementing technologies for bypassing Sanger confirmation in accordance with various embodiments.

FIG. 2 shows an exemplary Sanger bypassing system using machine learning models to make predictions on whether Sanger confirmation is required in accordance with various embodiments.

FIG. 3 shows a flowchart illustrating a process for utilizing Sanger bypassing techniques in genetic assays to determine which variants can be bypassed for Sanger sequencing confirmation and which variants requires Sanger sequencing confirmation in accordance with various embodiments.

FIG. 4 shows a block diagram of an exemplary machine learning pipeline comprising several subsystems that work together to train, validate, and implement one or more machine learning models in accordance with various embodiments.

FIGS. 5A-5D show block diagrams illustrating a multi-phase training and validation method for one or more machine learning models in accordance with various embodiments.

FIG. 6 shows a flowchart illustrating a process for training and validating one or more machine learning methods in accordance with various embodiments.

FIG. 7 is a flowchart illustrating how the Sanger bypass assay platform or system uses trained machine learning models to determine which variants qualify for Sanger sequencing bypass in accordance with various embodiments.

FIGS. 8A-8G shows density plots of quality features wherein FIGS. 8A-8D display features with positive effects that have a higher probability of being associated with false positive variants and FIGS. 8E-8G display features with negative effects that have a higher probability of being associated with true positive variants, in accordance with various embodiments.

FIGS. 9A-9F show comparisons of machine-learning model performance after training with balanced vs. imbalanced datasets and all features verse select high-impact features in accordance with various embodiments.

FIG. 10 shows a table comparing machine-learning model performance after training with balanced vs. imbalanced datasets and all features verse select high-impact features in accordance with various embodiments.

### TERMS

As used herein, the singular forms “a,” “an,” and “the” include plural references unless the context clearly dictates otherwise. For example, references to “the method” include one or more methods, and/or steps of the type described herein, which will become apparent to those persons skilled in the art upon reading this disclosure and so forth. Additionally, the term “nucleic acid” or “nucleic acid molecule” includes a plurality of nucleic acids, including mixtures thereof.

As used herein, the term “allele” refers to any alternative forms of a gene at a particular locus. There may be one or more alternative forms, all of which may relate to one trait or characteristic at the specific locus. In a diploid cell of an organism, alleles of a given gene can be located at a specific location, or locus (loci plural) on a chromosome. The genetic sequences that differ between different alleles at each locus are termed “variants,” “polymorphisms,” or “mutations.” The term “single nucleotide polymorphisms” (SNPs) can be used interchangeably with “single nucleotide variants” (SNVs). As used herein, the term “allele frequency” may refer to how often a particular allele appears within a population. The allele frequency may be calculated by dividing the number of times a specific allele appears in the population by the total number of alleles for that gene in the population. In some instances, the terms “allele frequency” and “population allele frequency” are used interchangeably.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1 percent, 1 percent, 5 percent, and 10 percent, etc. Moreover, the term terms “about,” “similarly,” “substantially,” and “approximately” are used to provide flexibility to a numerical range endpoint by providing that a given value may be slightly above or slightly below the endpoint without affecting the desired result.

As used herein, when an action is “based on” something, this means the action is based at least in part on at least a part of the something.

As used herein, the term “likely” refers to a probability range of about 80%-99% when describing the significance of an event. In some instances, “likely” is 95%-98%. For example, a “likely benign” variant has a 95%-98% chance of being benign, and a “likely pathogenic” variant has a 95%-98% chance of being pathogenic. Different ranges may be used for different events.

As used herein, the term “sample,” “biological sample,” “patient sample,” “tissue,” and “tissue sample” refer to any sample including a biomolecule (such as a protein, a peptide, a nucleic acid, a lipid, a carbohydrate, or a combination thereof) that is obtained from any organism including viruses, and the terms may be used interchangeably. Other examples of organisms include mammals (such as humans; veterinary animals like cats, dogs, horses, cattle, and swine; and laboratory animals like mice, rats and primates), insects, annelids, arachnids, marsupials, reptiles, amphibians, bacteria, and fungi. Biological samples include tissue samples (such as tissue sections and needle biopsies of tissue), cell samples (such as cytological smears such as Pap smears or blood smears or samples of cells obtained by microdissection), or cell fractions, fragments or organelles (such as obtained by lysing cells and separating their components by centrifugation or otherwise). Other examples of biological samples include blood, serum, urine, semen, fecal matter, cerebrospinal fluid, interstitial fluid, mucous, tears, sweat, pus, biopsied tissue (for example, obtained by a surgical biopsy or a needle biopsy), nipple aspirates, cerumen, milk, vaginal fluid, saliva, swabs (such as buccal swabs), or any material containing biomolecules that is derived from a first biological sample. In certain embodiments, the term “biological sample” as used herein refers to a sample (such as a homogenized or liquefied sample) prepared from a tumor or a portion thereof obtained from a subject.

As used herein, the terms “standard” or “reference,” refer to a substance which is prepared to certain pre-defined criteria and can be used to assess certain aspects of, for example, an assay. Standards or references preferably yield reproducible, consistent, and reliable results. These aspects may include performance metrics, examples of which include, but are not limited to, accuracy, specificity, sensitivity, linearity, reproducibility, limit of detection and/or limit of quantitation. Standards or references may be used for assay development, assay validation, and/or assay optimization. Standards may be used to evaluate quantitative and qualitative aspects of an assay. In some instances, applications may include monitoring, comparing and/or otherwise assessing a QC sample/control, an assay control (product), a filler sample, a training sample, and/or lot-to-lot performance for a given assay. As used herein, the term “reference genome” can refer to any particular known, sequenced or characterized genome, whether partial or complete, of any organism or virus which may be used to reference identified sequences from a subject.

Unless defined otherwise, all technical and scientific terms used herein have the same meaning as commonly understood by one of ordinary skill in the art to which this disclosure belongs. Although any methods and materials similar to or equivalent to those described herein can be used in the practice or testing of the application, the preferred methods and materials are now described.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

NGS has become very popular in clinical care and research due to its massively parallel sequencing abilities; however, Sanger sequencing remains the current standard of care for validating variants detected by NGS. This is despite several studies reporting that NGS is just as accurate, when appropriate quality thresholds are met, with concordance rates of >99% being reported for SNVs and indels in high-complexity regions. As a result, Sanger is taking on a new role where it is mostly being used to confirm variant calls in regions where NGS is unable to achieve sufficient depth coverage, regions with homology to other regions, regions with low complexity, repeat expansions, and methylation, or before variants are clinically reported.

The continued advancement in sequencing technologies has opened the door for the discovery and detection of even more disease-causing variants, allowing clinicians to better serve their patients. However, the increased demand for genetic screenings has exponentially increased the number of samples being submitted to the laboratories for testing. More specifically, in terms of scalability and throughput. NGS platforms are designed for high-throughput sequencing, enabling the analysis of large volumes of data efficiently. However, scaling up NGS operations to meet increased demand requires substantial investment in additional equipment, software, and skilled personnel. This expansion can be both costly and time-consuming. Furthermore, the verification of NGS results using Sanger sequencing, a more labor-intensive and lower-throughput method, can create bottlenecks in the workflow. The nature of Sanger sequencing verification processes can slow down the overall turnaround time for delivering conclusive results, thereby impacting the timely diagnosis and treatment of patients in clinical settings.

Additionally, the challenges extend to cost and resource allocation, data management, and quality control. While NGS is cost-effective for large-scale sequencing projects, the initial setup and ongoing maintenance of NGS infrastructure require significant financial outlay. For example, based on reported positivity rates, the transition from a catalog-based carrier screen to full-gene sequencing on the whole exome panel was projected to lead to at least a 2-fold increase in the number of positive cases tested in the laboratory and a substantial increase in the number of variants requiring sanger confirmation. The increase in the number of variants requiring Sanger sequencing confirmation will exponentially increase the cost of performing genetic testing, the labor involved in processing patient samples, the wet-lab reagents and consumables expended on the sequencing, and the turnaround time of results to clinics, impacting the quality of patient care and the cost of overall healthcare. Managing the vast amount of data generated by NGS necessitates robust bioinformatics pipelines and data storage solutions, which require specialized expertise and technology. Integrating and ensuring consistency between NGS and Sanger sequencing data can be complex and time-consuming. Moreover, maintaining high-quality standards for both NGS and Sanger sequencing involves rigorous quality control measures and adherence to regulatory standards, which can further complicate the workflow and increase the demand for meticulous oversight and standardization.

To address the increase in demand of variants requiring Sanger sequencing confirmation and other challenges, disclosed herein are techniques that utilize a two-tier machine learning process to select variants for bypassing Sanger sequencing in a genetic assay, so that the number of Sanger sequencing needed for confirming variants detected in the NGS is substantially decreased and the cost and turnaround time of the genetic assay for delivering results are substantially reduced. The disclosed techniques, which take both variant types and quality features into consideration to evaluate the true positive probability of a variant, overcome biases of only considering prior concordance data as a measure of confidence in determining which variants require confirmation. Experiments show that incorporating the disclosed techniques into genetic assays reduce the total number of variants previously requiring Sanger confirmation down to about 15% or less, significantly reducing the experimental overhead cost and turnaround time.

One illustrative embodiment of the present disclosure is directed to a computer-implemented method that includes performing next generation sequencing (NGS) on nucleic acid obtained from a biological sample of a subject to generate sequencing data; extracting information of a set of variants from the sequencing data, wherein the information of the set of variants comprises a type of each variant in the set of variants and one or more quality features of each variant in the set of variants; clustering the set of variants into one or more subsets of variants based on the type of each variant in the set of variants; generating, using a first machine learning model, a predicted status of each variant in at least one subset of the one or more subsets of variants based on the one or more quality features, wherein the predicted status is a presence status, an absence status, or an unknown status; generating, using a second machine learning model, a confirmatory status of each variant with the unknown status as the predicted status, wherein the confirmatory status is a presence status or an absence status; and performing Sanger sequencing on nucleic acid molecules comprising variants with the absence status as the predicted status or the confirmatory status to confirm an existence of the variants.

### II. Computing Environment

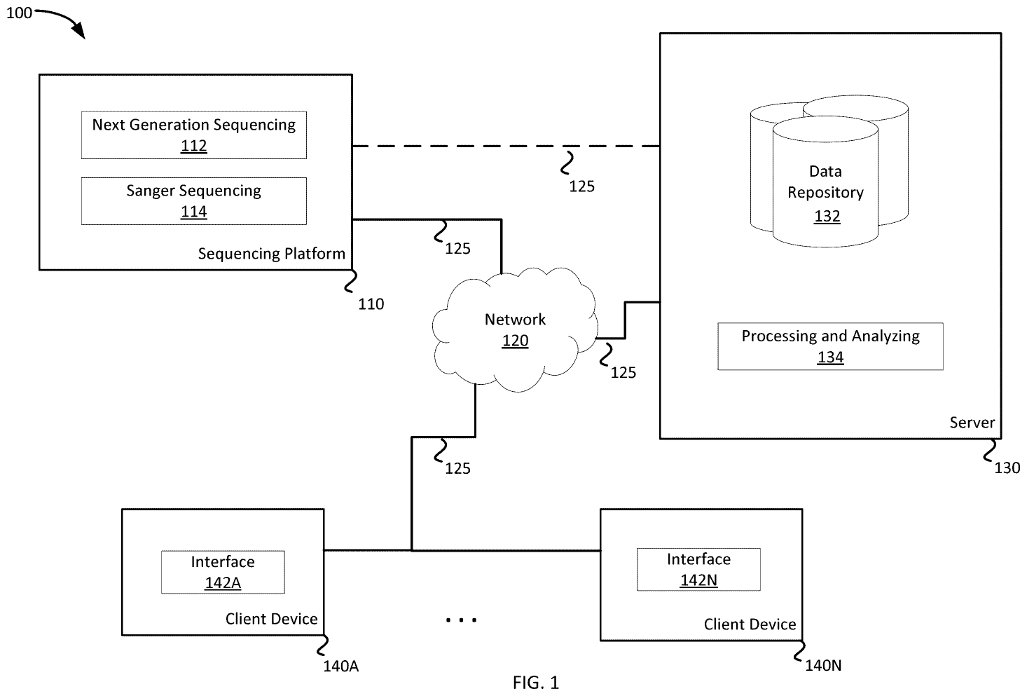

FIG. 1 shows an exemplary computing environment 100 for implementing technologies for bypassing Sanger confirmation in accordance with various embodiments. The computing environment 100 includes a sequencing platform 110, a network 120, a server 130, and one or more client devices 140A-N. Although FIG. 1 illustrates a particular arrangement of components of the computing environment 100, this disclosure contemplates any suitable arrangement of these components and additional components. As an example, and not by way of limitation, the server 130 and the sequencing platform 110 may be connected to each other directly and constitute a genetic assay platform, bypassing the network 120. As another example, the one or more client devices 140A-N, the server 130, and the sequencing platform 110 may be physically or logically co-located with each other in whole or in part. Moreover, although FIG. 1 illustrates a particular number of the components, this disclosure contemplates any suitable number of components (e.g., sequencing platforms 110, networks 120, servers 130, and client devices 140A-N). As an example, and not by way of limitation, the computing environment 100 may include two sequencing platforms 110, one network 120, multiple servers 130, and one client device 140A. In the computing environment 100, the term “unit” can be used interchangeably with “component” and can refer to software (e.g., a single piece of code), hardware (one or more physical components, such as a central processing unit (CPU), graphics processing unit (GPU), memory unit (RAM), storage unit (hard drive or SSD)), a device (e.g., a computer, a sequencer, a sequencing machine, or a combination thereof.

The sequencing platform 110 is configured to perform sequencing tasks including next generating sequencing (NGS) and Sanger sequencing. The sequencing platform 110 may operate fully automatically with loaded samples, or operate semi-automatically with the help of a practitioner. As illustrated in FIG. 1, the sequencing platform 110 may include two units: (1) an NGS unit 112 (e.g., a combination of laboratory equipment and an NGS sequencer such as the G4X Spatial Sequencer by Singular Genomics, the DNBSEQ-G400 Genetic Sequencer by Complete Genomics, the HiSeq 2500 or 300 Sequencing System by Illumina, or any like NGS Sequencing machine or system) capable of performing next-generation sequencing, and (2) a Sanger sequencing unit 114 (e.g., a combination of laboratory equipment and a genetic analyzer configured for Sanger sequencing and fragment analysis by capillary electrophoresis (CE) such as the SeqStudio Genetic Analyzer or Applied Biosystems 3730 Series Genetic Analyzer by ThermoFisher, the Spectrum Compact Capillary Electrophoresis (CE) System by Promega, or any like Sanger Sequencing machine or system) capable of performing Sanger sequencing. In some instances, the sequencing platform 110 may include additional units (not shown) beyond the NGS unit 112 and the Sanger sequencing unit 114, such as a third-generation sequencing (TGS) unit (e.g., performing single molecule real-time (SMRT) sequencing and/or nanopore sequencing), a pyrosequencing unit, an Ion Torrent sequencing unit, and/or a sequencing by ligation (SOLiD) unit. In some instances, the NGS unit 112 is capable of performing functions that are the same as or similar to those of one or more of the additional units.

The NGS unit 112 enables the rapid and high-throughput sequencing of complex genetic libraries, such as whole genomes, whole exomes, transcriptomes, or targeted regions of DNA or RNA. The NGS process performed using the NGS unit 112 begins with a nucleic acid extraction process to isolate high-quality DNA or RNA from a biological sample. This is followed by the preparation of a DNA or RNA library, where the genetic material is fragmented into smaller, more manageable pieces. This can be achieved through mechanical shearing, enzymatic digestion, or sonication. The fragmented DNA or RNA is then prepared for sequencing through the addition of sequencing adapters. These adapters are short sequences of DNA, e.g., double-stranded DNA sequences, that are ligated to the ends of the fragments, allowing them to bind to a flow cell. The flow cell is a specialized surface within the NGS instrument where sequencing takes place.

The NGS process performed using the NGS unit 112 may further include processing to ensure that the fragments are of the appropriate size and concentration for sequencing. This can include size selection, where fragments of a specific length are isolated using gel electrophoresis or magnetic beads. The prepared library may then be quantified and quality-checked using techniques such as quantitative PCR (qPCR) or bioanalyzer assays to ensure that it meets the requirements for sequencing. In some instances, wet-lab manual procedures are involved in the NGS process, including sample collection and preparation (e.g., DNA/RNA extraction), sample quantification and quality assessment (e.g., spectrophotometry, agarose gel electrophoresis), PCR and qPCR setup, library preparation for sequencing (e.g., fragmentation, adapter ligation, purification, size selection), cloning and transformation (e.g., ligation, bacterial transformation), cell culture (e.g., medium preparation, transfection), protein expression and purification (e.g., induction, chromatography), Western blotting (e.g., gel electrophoresis, antibody incubation), immunohistochemistry and immunocytochemistry (e.g., tissue sectioning, antibody staining), and microscopy (e.g., slide preparation, staining). In some instances, the procedures are performed automatically by automated systems and/or robotics.

Once the library is ready, the fragments are introduced into the NGS sequencer, where they are immobilized on the flow cell. The flow cell is a glass slide with a surface coated with oligonucleotides that are complementary to the adapter sequences on the DNA fragments. Generally, through a process called bridge amplification or clonal amplification, the fragments are amplified directly on the flow cell surface. During bridge amplification, each fragment bends over to hybridize with a nearby oligonucleotide on the flow cell, forming a bridge. DNA polymerase then extends the fragment, creating a double-stranded bridge. This process is repeated multiple times, resulting in clusters of identical DNA sequences that are spatially separated on the flow cell. These dense clusters amplify the signal that will be detected during sequencing, ensuring accurate and efficient data collection. It should be understood that different NGS platforms may have their own sequencing chemistries and technologies, generally involving the attachment of the library fragments to the solid surface, amplification to create clusters or colonies of identical sequences, and sequencing-by-synthesis or other methods to read the nucleotide sequence of each fragment.

By using these techniques, the NGS unit 112 can handle a vast number of fragments simultaneously, setting the stage for high-throughput sequencing. The immobilization and amplification steps are important for generating sufficient signal strength from each fragment, which is essential for the subsequent sequencing reactions. The entire process is automated and precisely controlled within the NGS sequencer, allowing for the parallel sequencing of millions to billions of fragments, and ultimately producing a massive amount of data that requires extensive computational resources for analysis.

The massive amount of sequencing data generated by the NGS unit 112 for each sample is immense and requires substantial processing to be useful. Each sample run of an NGS sequencer can produce terabytes of raw sequencing data, including the raw nucleotide sequence reads (e.g., millions to billions of sequence reads), quality scores for each base in each of the sequence reads, and metadata related to the sequencing run. This necessitates an intricate bioinformatics pipeline to transform the raw sequencing data into actionable genetic information. As part of the bioinformatics pipeline the NGS unit 112, the server 130, one or more other components of the sequencing platform 110, or any combination thereof analyze, process, and manage the sequencing data. The first stage in this pipeline is quality control, where tools like FastQC and Trimmomatic are employed to evaluate and enhance the quality of the raw sequence reads. This involves filtering out low-quality sequences and trimming adapter sequences that were added during library preparation. The sheer volume of data, often ranging from gigabytes to terabytes per sequencing run, requires high-performance computing hardware. Multi-core processors and ample RAM are used to handle the parallel processing and large memory requirements of these quality control operations efficiently.

Following quality control, the next step is read alignment, where the filtered, high-quality reads are mapped to a reference genome. This process is computationally intensive due to the complexity and size of the reference genome and the need to align millions to billions of short reads accurately. Alignment tools like BWA (Burrows-Wheeler Aligner) and Bowtie2 may be used for this purpose. High-core-count CPUs and substantial RAM are used to manage the parallel processing demands and to store the reference genome and intermediate data in memory. Fast storage solutions, such as SSDs (Solid State Drives), are used to minimize I/O bottlenecks during read alignment, ensuring swift data access and processing speeds.

Once the reads are aligned, the bioinformatics pipeline proceeds to variant calling, where genetic variants such as single nucleotide polymorphisms (SNPs) and insertions/deletions (indels) are identified. Variant callers such as GATK (Genome Analysis Toolkit) and FreeBayes may perform this task by comparing each aligned read to the reference genome and assessing the likelihood of different variants. This step is highly computationally demanding, requiring significant processing power to handle the large datasets and complex calculations. High-performance computing clusters or cloud-based solutions are often employed to distribute the computational load across multiple nodes and cores. When machine learning algorithms are integrated into this step (as described further herein), additional computational resources are needed to train and apply models that can improve the accuracy and efficiency of variant detection.

The final step in the pipeline is variant annotation, where identified variants are annotated to provide functional information, such as their impact on protein-coding genes or their association with known diseases. Annotation tools like ANNOVAR and SnpEff may be used to add this layer of information, drawing from large databases of genetic data. This step also requires significant computational resources, particularly when dealing with extensive datasets and complex annotations. High-performance CPUs, large amounts of RAM, and fast storage solutions are used to manage and process the data efficiently. To further enhance the processing capabilities, leveraging GPUs (Graphics Processing Units) for parallelizable tasks and ensuring sufficient and fast memory may be used to significantly improve performance. Additionally, scalable infrastructure, such as cloud-based platforms that offer flexible resource allocation, allows for accommodating larger datasets and more complex analyses as NGS technologies continue to advance. By optimizing these hardware aspects, the efficiency and speed of NGS data processing can be significantly enhanced, leading to faster and more accurate genetic analyses.

The Sanger sequencing unit 114 is configured to perform Sanger sequencing for determining or confirming the nucleotide sequence of DNA or RNA. A signal or instruction is received by the Sanger sequencing unit 114 to perform Sanger sequencing. The signal or instruction may come from the client devices 140A-N, the server 130, or the network 120. The signal or instruction may include variants or regions that the Sanger sequencing is performed. In some instances, the Sanger sequencing unit 114 includes software (e.g., Primer3, GeneDistiller, UCSC In-Silico PCR, Alamut Visual, SNPCheck, Vector VNTI Advance, or the like) to design primers to capture specific nucleic acid molecules. In some instances, the primers are universally tagged sequencing primers. In some instances, the Sanger sequencing unit 114 includes a PCR amplification component (e.g., FailSafe PCR System, HotStarTaq Master Mix Kit, or the like) to amplify nucleic acid molecules to be Sanger sequenced. The PCR process may include denaturation (e.g., separating the double-stranded DNA), annealing (e.g., binding of primers to the single-stranded DNA), and extension (e.g., synthesizing new DNA strands using DNA polymerase). The PCR process results in multiple copies of the nucleic acid molecules to ensure sufficient quantities of the nucleic acid molecules to be Sanger sequenced.

The Sanger sequencing unit 114 is also capable of synthesis of a complementary DNA strand using a single-stranded DNA template (or an RNA template), a DNA polymerase enzyme, and a mixture of normal deoxynucleotides (dNTPs) and chain-terminating dideoxynucleotides (ddNTPs). The ddNTPs are fluorescently or radioactively labeled and lack a 3′ hydroxyl group, which prevents further elongation of the DNA strand upon incorporation. By including a small proportion of ddNTPs in the reaction, a series of DNA fragments of varying lengths is generated, each terminating at a specific nucleotide. The resulting DNA fragments are then separated by size using capillary electrophoresis or polyacrylamide gel electrophoresis. In capillary electrophoresis, an electric field is applied to a capillary tube filled with a polymer matrix, which allows the fragments to migrate based on their size. Smaller fragments move faster through the capillary, while larger fragments move more slowly. As the fragments pass through a detector, the fluorescent or radioactive labels are detected, and the sequence of the DNA or RNA is determined by analyzing the order of the labeled fragments.

The Sanger sequencing performed at the Sanger sequencing unit 114 can be in either single direction (unidirectional) or bidirectional (forward and reverse). The output of the Sanger sequencing (e.g., the Sanger sequencing data) can be a chromatogram (e.g., a visual representation of the sequence of the nucleic acid molecule), detailed base calls, and/or associated quality scores. In some instances, the Sanger sequencing data can be compiled and interpreted using the sequencing platform 110 to reconstruct the original DNA or RNA sequence, validate of sequences obtained from the NGS unit 112, or detect variants in the biological materials. For example, the base calls may be compared to the variant calls generated at the NGS unit 112 and a determination is made regarding concordance between the NGS sequencing and the Sanger sequencing. If the base calls and the variant calls are consistent, the variant calls or the sequencing data are confirmed, and the data generated at the NGS unit 112 can be used for further analysis (e.g., determination of a disease or a somatic mutation). If there is a discordance between the base calls and the variant calls, the discordant variant made by the NGS unit 112 may be treated as a false positive (e.g., due to a sequencing error or artifact) and excluded from further analysis. In some instances, the discordant regions will be resequenced by Sanger sequencing or NGS sequencing to confirm the variant or sequence.

The Sanger sequencing data generated by the Sanger sequencing unit 114 can be further combined with the NGS sequencing data and sent to the processing and analyzing unit 134 for further analysis. The Sanger sequence data and/or the NGS sequencing data can also be sent to the client devices 140A-N for display (e.g., through interface 142A-N) or to the server 130 for analysis. Sanger sequencing remains a gold standard for its accuracy and reliability, particularly for smaller-scale sequencing tasks, diagnostic applications, and confirming genetic variations identified by other methods (e.g., the NGS method). In some instances, the Sanger sequencing confirmation may be substituted by another sequencing technique to perform a same or similar function of the Sanger sequencing unit 114 (e.g., to confirm the NGS sequencing result).

The network 120 is contemplated to be any type of networks familiar to those skilled in the art that support data communications using any of a variety of available protocols including without limitation TCP/IP (transmission control protocol/Internet protocol), SNA (systems network architecture), IPX (Internet packet exchange), AppleTalk®, and the like. Merely by way of example, the network 120 may be a local area network (LAN), networks based on Ethernet, Token-Ring, a wide-area network (WAN), a wireless local area network (WLAN), the Internet, a virtual network, a virtual private network (VPN), an intranet, an extranet, a public switched telephone network (PSTN), an infra-red network, a wireless network (e.g., a network operating under any of the Institute of Electrical and Electronics (IEEE) 1002.11 suite of protocols, Bluetooth®, and/or any other wireless protocol), and/or any combination of these and/or other networks.

Links 125 may connect the sequencing platform 110 or a unit thereof (e.g., the NGS unit 112 or the Sanger sequencing unit 114), the server 130 or a unit thereof (e.g., a data repository 132), and/or the client devices 140A-N to the network 120 or to each other. In some embodiments, one or more links 125 include one or more wireline (such as for example Digital Subscriber Line (DSL) or Data Over Cable Service Interface Specification (DOCSIS)), wireless (such as for example Wi-Fi or Worldwide Interoperability for Microwave Access (WiMAX)), or optical (such as for example Synchronous Optical Network (SONET) or Synchronous Digital Hierarchy (SDH)) links. In some embodiments, one or more links 125 each include an ad hoc network, an intranet, an extranet, a VPN, a LAN, a WLAN, a WAN, a WWAN, a MAN, a portion of the Internet, a portion of the PSTN, a cellular technology-based network, a satellite communications technology-based network, another link 125, or a combination of two or more such links 125. Links 125 need not necessarily be the same throughout the computing environment 100. A first link 125 may differ in one or more respects from another link 125.

In various instances, server 130 may be adapted to run one or more services or software applications that enable one or more embodiments described in this disclosure. In certain instances, server 130 may also provide other services or software applications that may include non-virtual and virtual environments. In some examples, these services may be offered as web-based or cloud services, such as under a Software as a Service (SaaS) model to users of the client devices 140A-N. The users operating the client devices 140A-N may in turn utilize one or more client applications to interact with the server 130 to utilize the services provided by these components (e.g., the data repository 132 and/or processing and analyzing unit 134). In the configuration depicted in FIG. 1, server 130 may include one or more components that implement the functions performed by server 135. These components may include software components that may be executed by one or more processors, hardware components, or combinations thereof. It should be appreciated that various different device configurations are possible, which may be different from the computing environment 100. The example shown in FIG. 1 is thus one example of a computing environment (e.g., a distributed system for implementing an example computing system) and is not intended to be limiting.

The server 130 may be comprised of one or more general purpose computers, specialized server computers (including, by way of example, PC (personal computer) servers, UNIX® servers, mid-range servers, mainframe computers, rack-mounted servers, etc.), server farms, server clusters, or any other appropriate arrangement and/or combination. The server 130 may include one or more virtual machines running virtual operating systems, or other computing architectures involving virtualization such as one or more flexible pools of logical storage devices that may be virtualized to maintain virtual storage devices for the server. In various instances, the server 130 may be adapted to run one or more services or software applications that provide the functionality described in the foregoing disclosure.

The computing systems in the server 130 may run one or more operating systems including any of those discussed above, as well as any commercially available server operating system. The server 130 may also run any of a variety of additional server applications and/or mid-tier applications, including HTTP (hypertext transport protocol) servers, FTP (file transfer protocol) servers, CGI (common gateway interface) servers, JAVA® servers, database servers, and the like. Exemplary database servers include without limitation those commercially available from Oracle®, Microsoft®, Sybase®, IBM® (International Business Machines), and the like.

In some implementations, the server 130 may include one or more applications to analyze and consolidate data feeds and/or data updates received from the sequencing platform 110 or the client devices 140A-N. As an example, data feeds and/or data updates may include, but are not limited to, in vivo feeds, in silico feeds, or real-time updates received from public studies, user studies, one or more third party information sources, and data streams (continuous, batch, or periodic), which may include real-time events related to sensor data applications, biological system monitoring, and the like. The server 130 may also include one or more applications to display the data feeds, data updates, and/or real-time events via one or more display devices (the interface 142A-N) of the client devices 140A-N.

The data repository 132 is a data storage entity (or sometimes entities) into which data has been specifically partitioned for an analytical or reporting purpose. The data repository 132 may be used to store data and other information generated or used by the sequencing platform 110, the processing and analyzing unit 134, and/or the client devices 140A-N. For example, the data repository 132 may be used to store data and information to be used as input into a genetic screening assay for generating a final variant call report. In some instances, the data and information relate to genetic sequences (genomic, exomic, and/or targeted) of nucleic acid molecules, high-confidence variants, information on variant type and clinical significance, population allele frequency, and other information used by the genetic assay. The data repository 132 may reside in a variety of locations including the sequencing platform 110, the server 130, or one or more of the client devices 140A-N. For example, a data repository used by the server 130 may be local to server 130 or may be remote from server 130 and in communication with server 130 via a network-based or dedicated connection of the network 120. The computing environment 100 may comprises multiple data repositories and each data repository 132 may be of different types or of the same type. In some embodiments, a data repository 132 may be a database which is an organized collection of data stored and accessed electronically from one or more storage devices of the server 130, and the server 130 may be configured to execute a database application that provides database services to other computer programs or to computing devices (e.g., the client devices 140A-N and the sequencing platform 110) within the computing environment 100. One or more of these databases may be adapted to enable storage, update, and retrieval of data to and from the database in response to SQL-formatted commands or like programming language that is used to manage databases and perform various operations on the data within them.

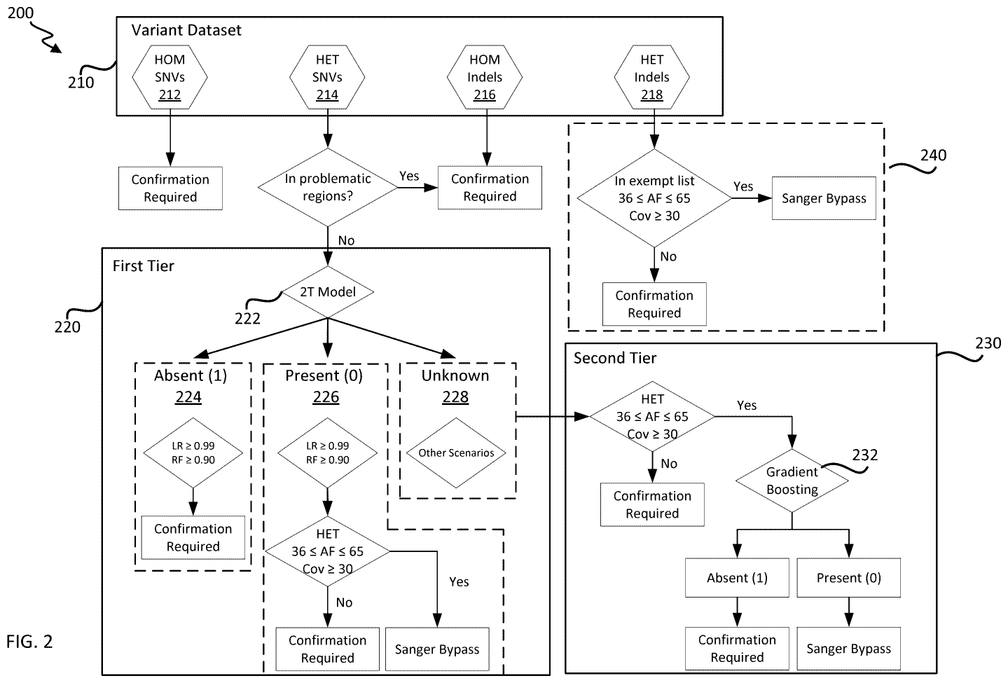

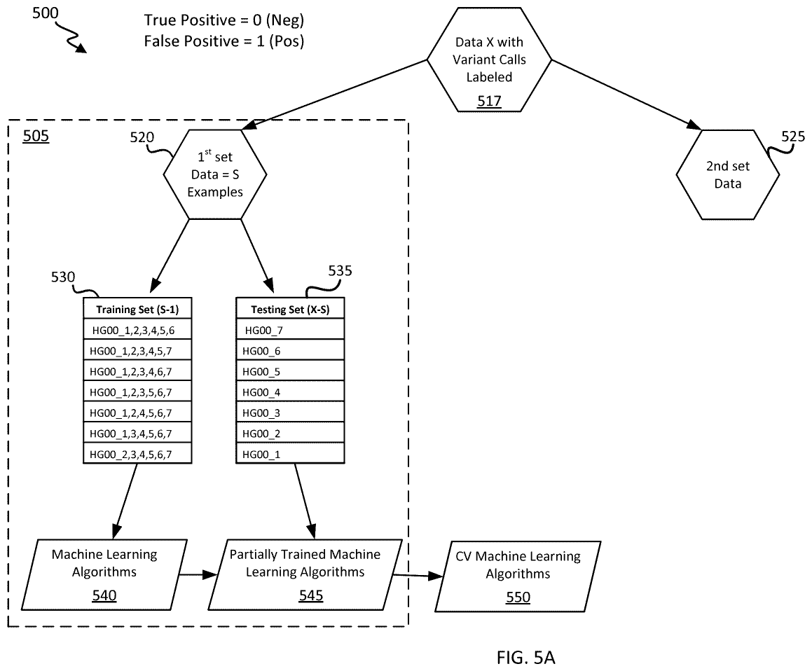

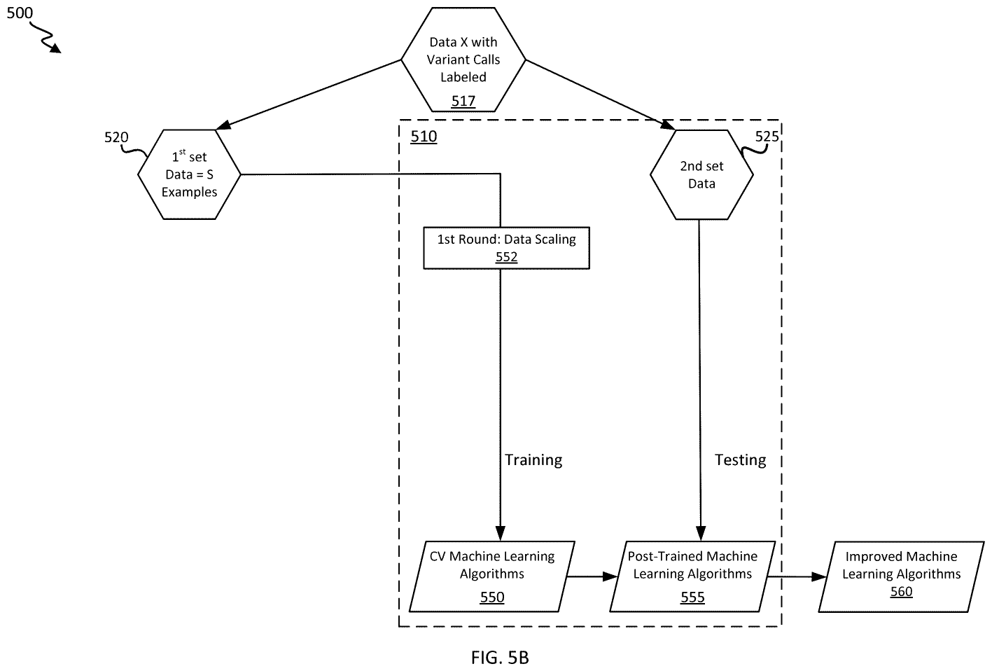

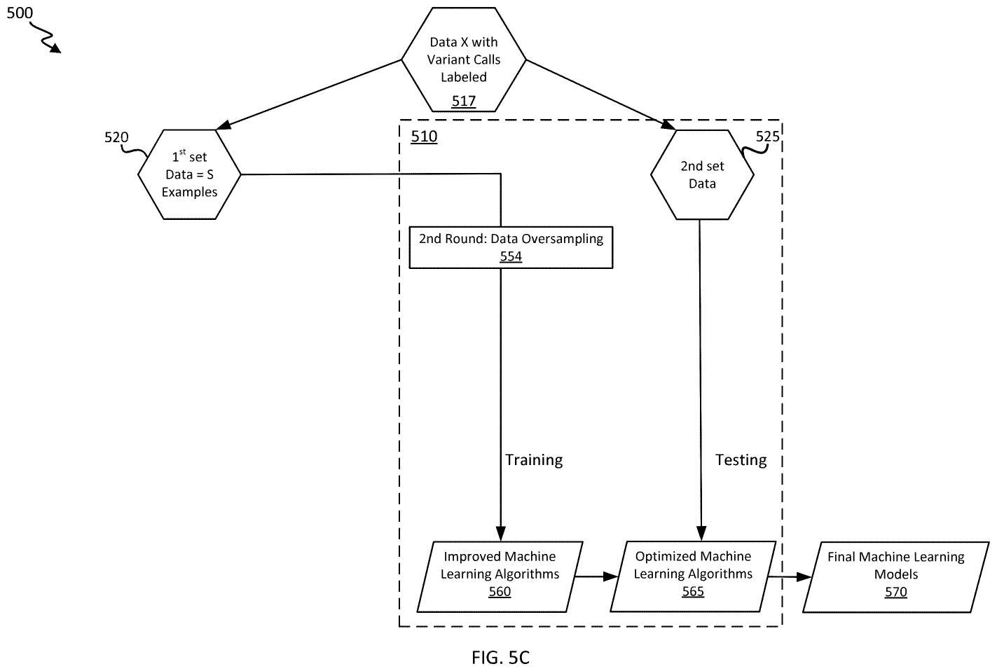

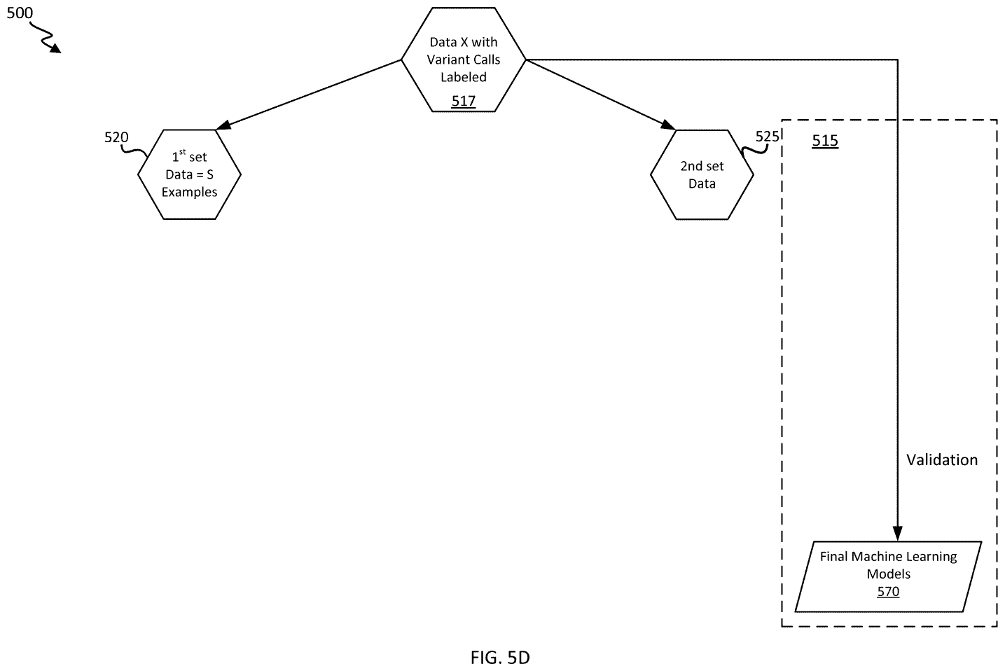

The processing and analyzing unit 134 is configured to process and analyze data (e.g., data stored in the data repository 132, data generated by the sequencing platform 110, or the data sent from the client devices 140A-N). The processing and analyzing unit 134 may further comprise a set of tools for the purpose of processing and analyzing data. For example, the processing and analyzing unit 134 may have a preprocessing tool capable of loading, processing, and saving data (e.g., accessed from the data repository 132) to be used by the preprocessing tool itself and/or a Sanger bypassing tool. The Sanger bypassing tool uses the processed data to identify a subset of segments that are subject to Sanger sequencing and another subset of segments that can bypass the Sanger sequencing. For example, the processing and analyzing unit 134 may be configured to perform the Sanger bypassing process 200 described with respect to FIG. 2 and/or a process 500 described with respect to FIGS. 5A-5D. In some instances, the processing and analyzing unit 134 is used together with the sequencing platform 110 and the data repository 132 to: (i) generate NGS read data for a biological sample for regions of interest (ROIs), (ii) extract variant information including types of variants and quality features from the NGS read data, (iii) cluster variants based on types of the variants, (iv) obtain population allele frequency information for the variants, (v) generate predicted status and/or confirmatory status for the variants, (vi) determine whether Sanger confirmation is required for regions comprising the variants, and (vii) perform the Sange sequencing on the required regions. The NGS read data and the Sanger sequencing data are used to obtain variant calls for the biological sample with improved accuracy and specificity, as described in detail with respect to FIGS. 2-4, 5A-5D, and 6-7. The processing and analyzing unit 134 may reside in a variety of locations including the sequencing platform 110, the server 130, and the client devices 140A-N. For example, a genetic assay comprising the processing and analyzing unit 134 may be local to the server 130 or may be remote from server 130 and in communication with the server 130 via a network-based or dedicated connection of the network 120.

The client device (e.g., the client device 140A) of the computing environment 100 is an electronic device including hardware, software, or embedded logic components or a combination of two or more such components and capable of interacting with the server 130 or a unit thereof (e.g., the data repository 132, the processing and analyzing unit 134) and the sequencing platform 110 or a unit thereof (e.g., the NGS unit 112, the Sanger sequencing unit 114), optionally via the network 120. The client devices 140A-N may include various types of computing systems such as portable handheld devices, general purpose computers such as personal computers and laptops, workstation computers, wearable devices, gaming systems, thin clients, various messaging devices, sensors or other sensing devices, and the like. These computing devices may run various types and versions of software applications and operating systems (e.g., Microsoft Windows®, Apple Macintosh®, UNIX® or UNIX-like operating systems, Linux or Linux-like operating systems such as Google Chrome™ OS) including various mobile operating systems (e.g., Microsoft Windows Mobile®, iOS®, Windows Phone®, Android™, BlackBerry®, Palm OS®). Portable handheld devices may include cellular phones, smartphones, (e.g., an iPhone), tablets (e.g., iPad®), personal digital assistants (PDAs), and the like. Wearable devices may include Ray-Ban Meta smart glasses, Meta Quest, Samsung Gear VR head mounted display (HMD), and other devices. The client devices 140A-N may be capable of executing various different applications such as various Internet-related apps, communication applications (e.g., E-mail applications, short message service (SMS) applications) and may use various communication protocols. This disclosure contemplates any suitable client device configured to generate and output product target discovery content to a user. For example, users may use the client devices 140A-N to execute one or more applications, which may generate one or more discovery or storage requests that may then be serviced in accordance with the teachings of this disclosure. The client devices 140A-N may provide an interface (e.g., a graphical user interface, e.g., the interface 142A) that enables a user of the client device 140A to interact with the client device 140A. The client devices 140A-N may also output information to the user via this interface 142A-N (e.g., displaying a variant call report). Although FIG. 1 depicts N client devices 140A-N, any number of client devices 105 may be supported.

The client devices 140A-N are capable of inputting data, generating data, and receiving data. For example, a user of a client device 140A may send out a request to perform a genetic assay using the interface 142A. The request may be sent out through the network 120 to the sequencing platform 110, and NGS or targeted NGS may be performed on a sample based on the request using the NGS unit 112. After the sequencing, the NGS reads or NGS data may be automatically sent to the server 130 through the network 120 for further processing. For example, the NGS data may be sent to the processing and analyzing unit 134 to generate variant calls and quality features of the variants using the set of tools of the processing and analyzing unit 134. Variant data (e.g., population allele frequencies of the variants) may be extracted or retrieved from the data repository 132 and sent to the processing and analyzing unit 134 together with the NGS data. Machine learning models may also be retrieved from the data repository 132 and provided to the processing and analyzing unit 134. Information may be further processed using the machine learning models and the processing and analyzing unit 134 to determine whether Sanger sequencing is required or can be bypassed. The Sanger bypassing/sequencing information may be sent back to the sequencing platform 110 to perform confirmatory sequencing using the Sanger sequencing unit 114. The Sanger bypassing/sequencing information may also be communicated to the user of the client devices 140A-N and the user may decide whether to perform the bypass/sequencing. The Sanger sequencing data may be sent back to the server 130 or the processing and analyzing unit 134 for subsequent analysis. For example, the NGS data and the Sanger sequencing data may be used together to determine sequences of the biological sample and make variant calling, and/or determine if a subject where the biological sample is obtained has developed or will develop a genetic condition (e.g., a disorder, a disease, or a cancer). The sample variant information and/or the disease diagnosis information may be transmitted to the client devices 140A-N via the network 120. The data (e.g., the NGS data, the Sanger sequencing data, the variant data, the quality features, and/or the population allele frequency information) may also be sent and stored in the data repository 132 for future analysis.

### III. Sanger Bypassing System

FIG. 2 shows a Sanger bypassing process 200 using machine learning models to make predictions on whether Sanger confirmation is required. The decision or prediction can be based on statistical likelihood of a variant being a true positive based on a set of measurable quality features extracted from NGS sequencing data. One goal of the Sanger bypassing process 200 is to reduce the number of true positive variants unnecessarily undergoing confirmatory Sanger sequencing and to instead focus resources, time and efforts in validating variants, such as false positive variants, that do not meet appropriate quality thresholds by NGS. This is achieved through the use of a computer-implemented program that answers a series of yes/no questions (such as variant type, variant location, the presence or absence of a variant in a dataset, whether or not the variants meet specified thresholds, etc.). The Sanger bypassing process 200 receives input from an annotated file that comprises quality features of one or more variant dataset 210, a first tier 220, a second tier 230, and an alternate pathway 240 for processing variants classified as heterozygous indels. The Sanger bypassing process 200 or one or more components of the Sanger bypassing process 200 may be executed as part of the sequencing platform 110, the server 130 of the computer environment 100, or on one or more of the client devices 140A-N described with respect to FIG. 1.

A variant dataset 210 comprising the quality features of one or more variants obtained from a genetic assay or an NGS assay (e.g., a WGS assay, a WES assay, or a targeted sequencing assay) is used as input for the Sanger bypassing process 200. Frequently, assays are performed on samples obtained from patients having genetic screening done to detect one or more genetic variants that can be benign, likely benign, variant of unknown significance, likely pathogenic or pathogenic. Variants are naturally occurring alterations (e.g., areas of the genome displaying changes in one or more nucleotide or chromosomal region) to the DNA sequence not found in a reference sequence. As way of example and not limitation, the types of variants that may be identified in the variant dataset 210 can include: homozygous (HOM) single nucleotide variants (SNVs) 212, heterozygous (HET) SNVs 214, HOM insertion-deletions (indels) 216, and/or HET indels 218. As described later in detail in FIGS. 5A-5D, the variant dataset may further comprises information (e.g., variant labeling) includes annotating variant calls in a group of samples with a truth label obtained from high-confidence variant data. In some instances, the variant dataset 210 is obtained from the sequencing platform 110 described with respect to FIG. 1. In some instances, the variant dataset 210 is obtained from the processing and analyzing unit 134 using sequencing data generated by the sequencing platform 110 described with respect to FIG. 1.

Variants whose sequencing read data indicate the same mutation is present in both alleles of the sample (e.g., HOM SNVs 212 and HOM indels 216) may be determined by the Sanger bypassing process 200 to require Sanger sequencing confirmation. Although not shown, a Sanger bypass assay platform or system can make predictions regarding the necessity of Sanger sequencing confirmation for HOM SNVs 212. When appropriate, an individual specialized in the field (e.g., a lab director, scientists, or the like) may decide if HOM SNVs 212 require Sanger sequencing confirmation based on experience.

In some embodiments, HET indels 218 are input into an alternate pathway 240 that will determine their eligibility for Sanger bypass based on a specific set of criteria. Sanger bypass eligibility for HET indels 218 can include (i) being in complete concordance with previously reported data and (ii) displaying allele frequency ranges consistent with heterozygous variant calls (e.g., allele frequencies (%) between 36-65 and read coverage greater than 30×). As described herein, allele frequency between 36-65 includes whole and rational values, for example, 36, 36.1, 36.5, 37, 38, 39, 40, 41, 42, 43, 44, 45, 46, 47, 48, 49, 50, 51, 52, 53, 54, 55, 56, 57, 58, 59, 60, 61, 62, 63, 64, and 65 and read coverage greater than or equal to 30×includes whole and rational values such as 30, 30.1, 30.5, 35, 40, 45, 50, 55, or greater without a maximum cutoff. Those HET indels 218 that meet the above criteria are eligible for Sanger bypass, while those HET indels 218 that do not meet the above criteria require Sanger sequencing confirmation. In some instances, the alternate pathway 240 is performed using the processing and analyzing unit 134 described with respect to FIG. 1.

Variants labeled as HET SNVs 214 undergo an inquiry as to whether they reside in problematic regions (e.g., areas with homology, low complexity, high complexity, low mappability, and repeat expansions). If yes, those HET SNVs 214 require Sanger sequencing confirmation. If no, they are input into the first tier 220 of the Sanger bypass assay platform or system, which comprises a 2T machine learning model 222 for predicting the statistical likelihood of a variant being a true positive or a false positive based on a set of measurable quality features and three decision branches: an absent variant branch 224, a present variant branch 226, and an unknown variant branch 228. As described herein and used interchangeably, an absent variant is synonymous with a false positive variant, while a present variant is synonymous with a true positive variant. Unknowns are variants that could not be classified as either absent or present by the 2T machine learning model 222. In some instances, the first tier 220 is performed using the processing and analyzing unit 134 or a client device (e.g., 140A) described with respect to FIG. 1. In some instances, the 2T machine learning model 222 is stored in and accessed from the data repository 132 described with respect to FIG. 1.

The 2T machine learning model 222 comprises at least two machine learning models, such as a logistic regression model trained and validated on a subset of high-impact (also referred to herein as “limited”) quality features and SOS balanced data and a random forest classifier trained and validated using all quality features and imbalanced data. Training and validating on all or subsets of quality features and/or balanced or imbalanced data are described with respect to FIGS. 5A-5D. Logistic regression modeling (sometimes referred to as logit regression) is a statistical model that is used to predict the probability of an event taking place. For example, the probability a variant is absent or present. During analysis, the logistic regression model uses limited quality features and balanced data with duplicated false positive variants added to it (e.g., SOS over sampling) to predict if a variant is absent (false positive) or present (true positive). The random forest classifier machine learning model uses the raw, imbalanced labeled variant data with all the quality features to construct a multitude of decision trees and make a prediction based on the class selected by the most trees. For example, if the majority of the decision trees found the variant in question to be a false positive, the model would report false positive for the variant.

To determine which of the three decision branches (the absent variant branch 224, the present variant branch 226, or the unknown variant branch 228) the HET SNVs 214 should be classified as, the 2T machine learning model 222 combines the logistic regression model and random forest classifier model using concordant predictions at fixed probability rates. What this means is that both models predict the same, or concordant, class (absent or present) for the variant at their respective predetermined probability rates, where the confidence threshold for the logistic regression is set to greater than or equal to 0.99 and the confidence threshold for the random forest classifier model is set to greater than or equal to 0.9. As described herein, greater than or equal to a value, for example 0.9, comprises the values 0.9, 0.91, 0.92, 0.93, 0.94, 0.95, 0.96, 0.97, 0.98, 0.99, and 1.0.

Concordance describes the proportion of shared attributes between two systems, for example a logistic regression model and a random forest classifier model, given that one system already possesses the attribute (e.g., variant classified as absent). An attribute is said to be concordant if both systems have the attribute and discordant if one system has the attribute and the other does not. For example, if both the logistic regression model and the random forest classifier model predict a variant to be absent, that variant is considered concordant between the two models and will follow the absent variant branch 224. On the other hand, if the two models reach a discordant, or disagreeing, decision, the variant will traverse down the unknown variant branch 228.

After the HET SNVs 214 are predicted as either absent (false positive variants), present (true positive variants), or unknown by the 2T machine learning model 222, their eligibility for Sanger bypass will be determined based on their corresponding decision branch. HET SNVs 214 that are predicted absent proceed using the absent variant branch 224 and will be confirmed by Sanger sequencing. Those HET SNVs 214 predicted to be present will follow the present variant branch 226. To prevent false positive variants from being incorrectly Sanger bypassed, HET SNVs 214 classified as present also have to pass a sequencing quality check point to determine if they pass or fail quality criteria (e.g., whether their population allele frequency (%) is between about 36 and about 65, whether the variants overlap with technically complex regions, and they have an average read coverage of greater than 30×). Different criteria may be designed based on different genetic assays or laboratory needs. Present HET SNVs that fail to pass these quality criteria will require Sanger sequencing confirmation, while those that do pass the quality criteria qualify for Sanger bypass.

Allele frequency refers to the count of reads supporting the mutation divided by the total read coverage for that locus. A lower allele frequency is indicative of an allele that is less likely to be present and would therefore need to be confirmed by Sanger sequencing. In some embodiments, allele frequency refers to population allele frequency obtained based on variant data obtained from a public database. Technically complex regions can include regions of homology and repetitive sequence tracts. The deeper the sequencing or coverage indicates that more sequencing reads are present at that given region.

When the 2T machine learning model 222 is unable to classify the HET SNVs as absent or present (e.g., the two models reach a discordant decision and/or the confidence thresholds of either or both models is not meet), the variant is classified as unknown and is passed to the second tier 230 of the Sanger bypass assay platform or system. The purpose of the second tier 230 is to use a different machine learning model to try again to predict the presence or absence of the unknown variants. This approach aims to prevent as many false positives as possible from being incorrectly bypassed for Sanger sequencing confirmation as well as rescue as many true positives as possible from unnecessary Sanger confirmation. The first step of the second tier 230 is to confirm the sequencing quality of the unknown variants using the allele frequency and read coverage thresholds described above in the present variant branch 226. If the unknown variant does not meet the allele frequency and/or read coverage thresholds, the unknown variant will require Sanger sequencing confirmation. If the unknown variant does pass the thresholds, it will be input into a third machine learning model. The third machine learning model comprises a gradient boost model 232 that uses the raw, imbalanced labeled variant data and all quality features to predict if the unknown variant if absent and will require Sanger sequencing confirmation or present and is eligible for Sanger bypass. In some instances, the second tier 230 is performed using the processing and analyzing unit 134 or a client device (e.g., 140A) described with respect to FIG. 1. The second tier 230 may be performed using the same or different processing and analyzing unit 134, the same client device (e.g., 140A), or a different client device (e.g., 140N) from the client device where the first tier is performed. In some instances, the gradient boost model 232 is stored in and accessed from the data repository 132 described with respect to FIG. 1.

For the variants classified as absent based on their quality features by the Sanger bypass assay platform or system, Sanger sequencing confirmation is required. Briefly, Sanger sequencing specifically utilizes chain-termination where specialized DNA bases (dideoxynucleotides or ddNTPs) are randomly incorporated into a growing DNA chain of nucleotides (A, C, G, T) generating different length DNA fragments. Capillary electrophoresis separates the fragments by size and a laser is used to excite the unique fluorescence signal associated with each ddNTP. The fluorescence signal captured shows which base is present at a given location of the target region being sequenced. The Sanger sequencing can be performed using the sequencing platform 110 described with respect to FIG. 1.

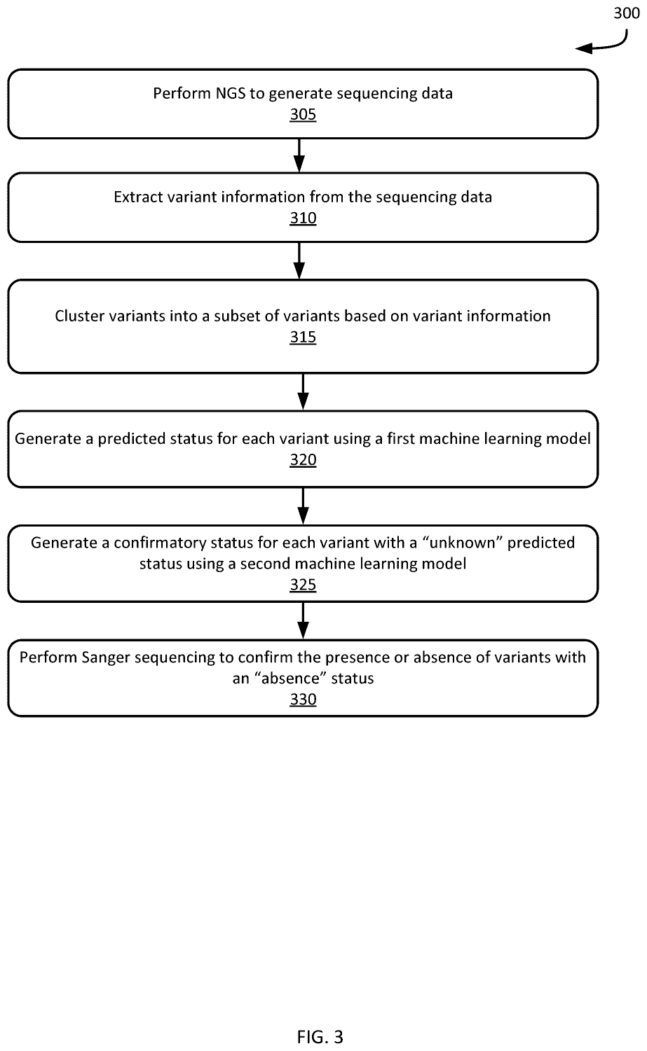

FIG. 3 shows a flowchart illustrating a process 300 for utilizing Sanger bypassing techniques in genetic assays to determine which variants can be bypassed for Sanger sequencing confirmation and which variants requires Sanger sequencing confirmation in accordance with various embodiments. The processing depicted in FIG. 3 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 3 and described below is intended to be illustrative and non-limiting. Although FIG. 3 depicts the various processing steps occurring in a particular sequence or order, this is not intended to be limiting. In certain alternative embodiments, the steps may be performed in some different orders, or some steps may also be performed in parallel.

At block 305, NGS is performed on nucleic acid obtained from a biological sample of a subject to generate sequencing data. The NGS can be performed using the sequencing platform 110 described with respect to FIG. 1. The NGS may be WGS, WES, or targeted sequencing. The nucleic acid can be DNA segments or molecules, or RNA segments or molecules. In some embodiments, the biological sample is collected from a subject when a clinical genetic assay is administered to be performed to the subject. As used herein, the term “genetic assay,” “genetic testing,” “clinical genetic assay,” or “genetic screening test” refers to a process of testing individuals or populations for specific genetic traits, mutations, or abnormalities that may indicate a predisposition to certain diseases, conditions, or inherited disorders, including but not limited to the followings: prenatal genetic screening tests (e.g., non-invasive prenatal testing (NIPT) or non-invasive prenatal screening (NIPS), first trimester screening, second trimester screening (quad screen), carrier screening, amniocentesis, and chorionic villus sampling (CVS)); newborn screening tests (e.g., the heel prick test (Guthrie test)); cancer genetic screening (e.g., BRCA1 and BRCA2 testing, Lynch syndrome screening, and FAP (familial adenomatous polyposis) testing); cardiovascular genetic screening (e.g., familial hypercholesterolemia testing and hypertrophic cardiomyopathy testing); neurological genetic screening (e.g., Huntington's disease testing and Alzheimer's disease genetic testing); metabolic and other genetic disorders screening (e.g., cystic fibrosis testing, thalassemia and sickle cell disease testing, and hemochromatosis testing); pharmacogenomic testing includes (e.g., cytochrome P450 testing); ancestry and health-related genetic screening; rare disease screening (e.g., exome sequencing and whole genome sequencing); genetic screening for specific populations; carrier screening; and prenatal and preconception screening (e.g., expanded carrier screening). In some embodiments, “genetic assay” refers to “carrier screening.” In some embodiments, the genetic assay excludes any assay performed on samples obtained from a pregnant female.

At block 310, variant information is extracted from the sequencing data using a pre-programmed computer script. The extraction can be performed using the sequencing platform 110, the server 130, or the client devices 140A-N described with respect to FIG. 1. The variant information includes variant types and quality features of the variants. The variant types may include: homozygous (HOM) single nucleotide variants (SNVs), heterozygous (HET) SNVs, HOM insertion-deletions (indels), and HET indels. The variant types may further include SVs, CNVs, translocations, and tandem repeats. The quality features may include read count (the number of times a DNA segment is sequenced), read coverage (the average number of times a nucleotide is read), frequency (the proportion of reads supporting a variant), allele frequency, forward count and reverse count (indicating the number of reads aligned in the forward and reverse directions, respectively), forward/reverse ratio, average quality, probability (the likelihood of a true variant), read position probability, read direction probability, homopolymer sequences and their length, and complex regions (e.g., repetitive elements or high GC content) (see Table 1). Tools or scripts used for the variant calling may include GATK (Genome Analysis Toolkit), FreeBayes, Samtools, BCFtools, VarScan, Platypus, Strelka, DeepVariant, and the like. The choice of tools may depend on the sequencing data, clinical or laboratory design, and/or analysis requirements.

At block 315, variants are clustered into subsets of variants based on the variant information. The clustering can be performed using the sequencing platform 110, the server 130, or the client devices 140A-N described with respect to FIG. 1. For example, as shown in FIG. 2, the variants are clustered into subsets of HOM SNVs 212, HET SNVs 214, HOM indels 216, and HET indels 218. In some embodiments, only the HET SNVs are subject to further processing and all other variants are required or recommended to be Sanger confirmed. In some embodiments, the HET indels are further filtered based on predetermined criteria. For example, if the allele frequency for a HET indel is about 36-65 with a coverage of greater than or equal to 30×, the HET indel can bypass Sanger sequencing. Otherwise, the Sanger confirmation is still required.

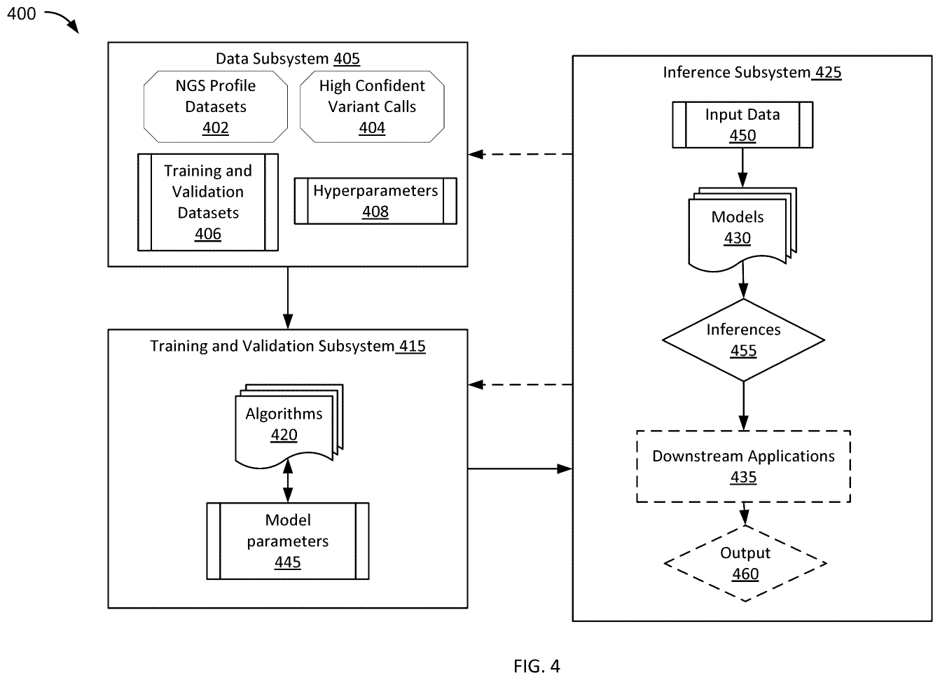

At block 320, a predicted status for each variant in a cluster is generated using a first machine learning model. The generation can be performed using the processing and analyzing unit 134 of the server 130, or on the client devices 140A-N described with respect to FIG. 1. The first machine learning model may be the 2T machine learning model 222 described with respect to FIG. 2. The cluster may be subset of the HET SNVs. Before performing the prediction using the first machine learning model, a filtering step may be performed. For example, variants in “problematic” regions may be excluded from further analysis and require to be Sanger confirmed. The problematic regions may include complex regions or regions with homology, low complexity, and/or repeat expansions. The first machine learning model may be a logistic regression model, a random forest, an EasyEnsemble model, an AdaBoost model, or a gradient boosting model. Other examples of algorithms used in the first machine learning model include, without limitation, linear regression, decision tree, Support Vector Machines, Naives Bayes algorithm, Bayesian classifier, linear classifier, K-Nearest Neighbors, K-Means, dimensionality reduction algorithms, grid search algorithm, genetic algorithm, and Artificial Neural Networks or large language models (LLMs) such as convolutional neural network (“CNN”), an inception neural network, a U-Net, a V-Net, a residual neural network (“Resnet”), a transformer neural network, a recurrent neural network, a Generative adversarial network (GAN), or other variants of Deep Neural Networks (“DNN”) (e.g., a multi-label n-binary DNN classifier or multi-class DNN classifier). In some embodiments, the first machine learning model is a model combining logistic regression and random forest. In some embodiments, the first machine learning model is trained using reference NGS data of reference samples obtained from a database and labeled variant data obtained from the database. Details about training and development of the machine-learning models are described in detail with respect to FIG. 4 and FIGS. 5A-5D.

The predicted status may be generated based on the quality features extracted at block 310. In some embodiments, the predicted status include (i) “presence,” which confirms the variant is a true positive and does not require further analysis or Sanger confirmation status, (ii) “absence,” which predicts the called variant is a false positive and requires performing Sanger sequencing to confirm if the variant is truly present or not, and (iii) “unknown,” which means the predicted status is insufficient to determine bypassing or performing Sanger sequencing. The variants with “unknown” status are subject to further analysis (e.g., block 325). In some embodiments, when the predicted status is “present,” a further filtering step may be performed to further the accuracy of the Sanger bypassing pipeline. For example, if the allele frequency for the variant with the “presence” label is about 36-65 with a coverage of greater than or equal to 30×, the variant can bypass Sanger sequencing. Otherwise, the Sanger confirmation is still required.

At block 325, a confirmatory status is generated for each variant with an “unknown” predicted status using a second machine learning model. The generation can be performed using the processing and analyzing unit 134 of the server 130, or on the client devices 140A-N described with respect to FIG. 1. The second machine learning model may be the gradient boosting model 232 described with respect to FIG. 2. Before performing the confirmation using the second machine learning model, a filtering step may be performed. For example, variants with the allele frequency about 36-65 and a coverage greater than or equal to 30× may be subject to further analysis; otherwise, Sanger confirmation is required. The second machine learning model may be a logistic regression model, a random forest, an EasyEnsemble model, an AdaBoost model, or a gradient boosting model. Other examples of algorithms used in the second machine learning model include those can be used in the first machine learning model discussed at block 320. The confirmation status may be generated based on the quality features extracted at block 310. In some embodiments, the confirmation status include (i) “presence,” which confirms the variant is a true positive and does not require further analysis or Sanger confirmation status, and (ii) “absence,” which confirms the variant may be a false positive and requires performing Sanger sequencing to confirm if the variant is truly present or not. In some embodiments, the second machine learning model is trained using reference NGS data of reference samples obtained from a database and labeled variant data obtained from the database. Details about training are illustrated with respect to FIG. 4 and FIGS. 5A-5D.

At block 330, the Sanger sequencing is performed on regions comprising variants with an “absence” status to validate the presence or absence of the variants. The Sanger sequencing can be performed using the sequencing platform 110 described with respect to FIG. 1. In some embodiments, targeted nucleic acid molecules are subjected to a sequencing reaction, which includes a mixture of normal dNTP) and fluorescently labeled ddNTPs. The ddNTPs are incorporated into the growing DNA strand during replication but cause termination when they are added, resulting in DNA fragments of varying lengths. Each ddNTP is labeled with a different fluorescent dye that corresponds to one of the four nucleotide bases (A, T, C, G), allowing for differentiation. The resulting DNA fragments are then separated by size using capillary electrophoresis. As the fragments pass through a laser, the fluorescent labels are excited and emit light at specific wavelengths, which is detected and recorded by a sensor. The sequence of the original DNA strand is determined by analyzing the order of the fluorescent signals, producing a chromatogram that represents the nucleotide sequence of the target nucleic acid molecules.

In some embodiments, a testing report for the subject is generated based on the sequencing data, the variant information, the predicted status, the confirmatory status, and/or results of the Sanger sequencing. The testing report may be displayed to a user of a client device (e.g., client device 140A). In some embodiments, the testing report is encrypted.

### IV. Training and Using Machine Learning Models in a Sanger Bypassing System

FIG. 4 shows a block diagram of a machine learning pipeline 400 comprising several subsystems that work together to train, validate, and implement one or more machine learning models in accordance with various embodiments. The machine learning pipeline 400 may be executed as part of the server 130 (e.g., the processing and analyzing unit 134) of the computer environment 100 described in FIG. 1. The machine learning pipeline 400 comprises a data subsystem 405 for collecting, generating, preprocessing, and labeling of data (e.g., from NGS profile datasets 402, high-confident variant calls 404, or training and validation datasets 406), training and validation subsystem 415 that facilitates the training and validation of one or more machine learning algorithms 420, and inference subsystem 425 for deploying and implementing one or more trained machine learning models 430 independently or in combination with one or more other downstream applications 435 (e.g., systems or services) for downstream processes. Each of the subsystems or operations of the machine learning pipeline 400 may be implemented by the server 130 or part of the server 130 described with respect to FIG. 1. In some embodiments, the machine learning pipeline 400 or one or more operations of the machine learning pipeline 400 are performed on one or more of the client devices 140A-N described with respect to FIG. 1.

As used herein, machine learning algorithms (also described herein as simply algorithm or algorithms) are procedures that are run on datasets (e.g., training and validation datasets) and perform pattern recognition on datasets, learn from the datasets, and/or are fit on the datasets. Examples of machine learning algorithms include linear and logistic regression, decision trees, artificial neural networks, k-means, and k-nearest neighbor. In contrast, machine learning models (also described herein as simply model or models) are the output of the machine learning algorithms and are comprised of model data and a prediction algorithm. In other words, the machine learning model is the program that is saved after running a machine learning algorithm on training data and represents the rules, numbers, and any other algorithm-specific data structures required to make inferences. For example, a linear regression algorithm may result in a model comprised of a vector of coefficients with specific values, a decision tree algorithm may result in a model comprised of a tree of if-then statements with specific values, or neural network, backpropagation, and gradient descent algorithms together result in a model comprised of a graph structure with vectors or matrices of weights with specific values.

### (A) Data Subsystem

Data subsystem 405 is used to collect, generate, preprocess, and label data to be used to train and validate one or more machine learning algorithms 420. The data collection can include exploring various data sources such as public datasets, private data collections, or real-time data streams, depending on a project's needs. In some instances, a data source is a public or online repository of information or examples pertinent to a general or target domain space. Many domains have publicly available datasets provided by governments, universities, or organizations. For example, many government and private entities offer datasets on healthcare, environmental data, and more through various portals. For proprietary needs, data might be available through partnerships or purchases from private companies that specialize in data aggregation. In other instances, a data source is a private repository of information or examples pertinent to a general or target domain space. Once a data source is identified, data subsystem 405 can be used to collect data through appropriate methods such as downloading from online repositories, web scraping, using APIs for real-time data, creating datasets through surveys and experiments, or by running assays. The acquired raw data may be further preprocessed to generate the training and validation datasets 406.

In some instances, raw data may be generated as opposed to being collected or acquired. Data generating may comprise data synthesis and/or data augmentation. Different data synthesis and/or data augmentation techniques may be implemented by the data subsystem 405 to generate data to be used for the training and validation subsystem 415. Data synthesizing involves creating entirely new data points from scratch. This technique may be used when real data is insufficient, too sensitive to use, or when the cost and logistical barriers to obtaining more real data are too high. The synthesized data should be realistic enough to effectively train a machine learning model, but distinct enough to comply with regulations (e.g., copyright and data privacy), if necessary. Techniques such as Generative Adversarial Networks (GANs) or Variational Autoencoders (VAEs) may be used to generate new data examples. These models learn the distribution of real data and attempt to produce new data examples that are statistically similar but not identical. Data augmentation, on the other hand, refers to techniques used to artificially expand the size of a dataset by creating modified versions of existing data examples. The primary goal of data augmentation is to increase variation in the data in order to make the model more robust to variations it might encounter in the real world, thereby improving its ability to generalize from the training data to unseen data. This is especially common in image and speech recognition tasks but is applicable to other data types as well. For images, data augmentation may include rotations, flipping, scaling, or altering the color/lighting conditions. For text, data augmentation may include synonyms replacement, back translation, or sentence shuffling. For audio, data augmentation may include changes made to pitch, speed, or background noise.

In some embodiments, the NGS profile datasets 402 may be generated from one or more NGS assays (e.g., WES assays such as the Twist exome panel or any other gene panel) used for genetic screening. The NGS profile datasets 402 comprise raw sequence read data (raw data) from a subject's genome, exome, or targeted regions of interest, processed sequence read data, quality features that describe characteristics of the sequence read data, or any combination thereof. In some instances, the NGS profile datasets 402 are acquired from a clinical laboratory or health care system (e.g., a genetic screening system, a patient record system, clinical trial testing system, and the like). In some instances, the NGS profile datasets 402 are acquired from a data storage structure such as a database, a laboratory or hospital information system, or any other modality for acquiring NGS assay results for subjects. In other instances, the NGS profile datasets 402 are acquired directly from a genetic screening assay system or clinical trial testing system that performs sequencing (e.g., NGS). The data subsystem 105 can be configured to provide the NGS profile datasets 402 to a data preprocessing module. One of ordinary skill in the art should understand that if an end user wants to apply the techniques described herein to WGS, WES, or targeted regions of interest, then the machine learning models described herein would need to be trained and tested using datasets in a similar manner as described herein with respect to NGS profile datasets.

When the NGS profile datasets 402 are directly acquired from a genetic assay, the data can be provided as raw read files (e.g., FASTQ files), alignment files (e.g., BAM files), variant files (e.g., VCF files), and the like. In more detail, machine sequencing of samples produces a large number of short reads deposited in a file with associated quality scores. These reads are typically aligned to a reference sequence, such as a reference genome, and the results are deposited in an alignment file (e.g., BAM). Variants are called and their properties relevant to the sequence (e.g., type of variant) are annotated and deposited in a variant file (e.g., variant call format (VCF)). In some instances, the NGS profile datasets 402 may be analyzed by outsourced sequencing/bioinformatic software (e.g., CLCbio and QIAGEN CLC Genomics Workbench) that generate annotated files (xml files).

The high-confidence variant calls 404 (also referred to as gold standards or benchmark variant calls) may be accessed from publicly available data sources (e.g., NCBI, GIAB, etc.). Moreover, these datasets may be generated by multiple sequencing technologies (Sanger, NGS, and the like) used for validating variant calling in pipelines, such as a Sanger bypass pipeline described herein. The high-confidence variant calls 404 can comprise small variants and variants in more difficult regions of the genome and are stored in VCF files for integration into variant calling pipelines.

As described herein, variants comprise naturally occurring alterations to the DNA sequence not found in the reference sequence, and the alterations can be classified as benign, likely benign, variant of unknown significance, likely pathogenic or pathogenic. Moreover, variants can comprise both germline variants (e.g., variants present in all the body's cells) and somatic variants (variants that arise during the lifetime of an individual). Examples of variants include small structural variants (less than 50 base pairs) such as single nucleotide variants (SNVs), single nucleotide polymorphisms (SNPs) and small structural variants (SVs) (e.g., deletions, insertions, insertions and deletions, sometimes referred to as indels) and larger (greater than 50 base pairs) SVs such as chromosomal rearrangements (e.g., translocations and inversions). In some embodiments, SNVs/SNPs are the result of single point mutations that can cause synonymous changes (nucleotide change does not alter the encoded amino acid), missense changes (nucleotide change does alter the encoded amino acid), or nonsense changes (resulting amino acid change converts the encoded codon to a stop codon). Further, variants can occur in both coding and non-coding regions of the genome and detected by NGS technologies.

Preprocessing may be implemented using data subsystem 405, serving as a bridge between raw data acquisition and effective model training. The primary objective of preprocessing is to transform raw data into a format that is more suitable and efficient for analysis, ensuring that the data fed into machine learning algorithms is clean, consistent, and relevant. This step can be useful because raw data often comes with a variety of issues such as missing values, noise, irrelevant information, and inconsistencies that can significantly hinder the performance of a model. By standardizing and cleaning the data beforehand, preprocessing helps in enhancing the accuracy and efficiency of the subsequent analysis, making the data more representative of the underlying problem the model aims to solve.

Preprocessing may be performed using a processor (e.g., a CPU, GPU, TPU, FPGA, the like, or any combination thereof), memory, and storage that operates software or computer program instructions (e.g., TensorFlow, PyTorch, Keras, and the like) to execute arithmetic, logic, input and output commands for processing acquired data. One operation of the processor is to generate a labeled variant dataset for training, validating, and/or testing one or more machine learning models. To accomplish this, the processor annotates variant calls within the NGS profile datasets 402 with true positive labels based on the high-confidence variant calls 404. For example, the high-confidence variant calls 404 have all known variants labeled as truths. If the NGS profile datasets 402 also have the same known variant present, the variant is labeled as present or true positive. On the other hand, if the NGS profile dataset 402 includes variants not found in the high-confidence variant calls 404, these variants are labeled as absent or false positives. The labeled variant dataset can comprise small structural variants such as SNVs/SNPs, indels, and the like.

Further, the processor measures weights, coefficients, and importance values for all quality features, a subset of quality features, or both by scaling acquired datasets (e.g., the NGS profile datasets 402 or the labeled variant dataset described above). Data scaling comprises adjusting the features in a machine learning model so that all features are on a relatively similar scale close to normal distribution. Further, data scaling also helps to identify quality features that have the highest impact on the performance of the machine learning models being assessed (e.g., false positive capture rates and true positive flag rates) and remove redundant quality features. The processor performs scaling on the NGS profile datasets 402 to determine the relative contribution of each feature to the associated true positive or false positive label. Methods for data scaling include: MinMaxScaler, RobustScaler, StandardScaler, Normalizer, and any other methods known to one of skill in the art.

Given the high degree of accuracy of NGS bioinformatic pipelines and variant calling tools to identify small variants, there is a much smaller proportion of false positive variants in the NGS profile datasets 402. To overcome this, the processor can implement oversampling and/or undersampling techniques to adjust the class ratio of the dataset (e.g., the ratio between true positive variants and false positive variants). Oversampling techniques can include random oversampling, simple over sampling (SOS), synthetic minority oversampling (SMOTE), adaptive synthetic sampling (ADASYN), augmentation, and the like. Techniques used for undersampling include random undersampling, cluster, tomek links, undersampling with ensemble learning, and the like.

An example of preprocessing, data may be collected from the NGS profile datasets 402 and the high-confident variant calls 404 to generate training and validation datasets 406. In the instance that machine learning pipeline 400 is used for supervised or semi-supervised learning of machine learning models, labeling techniques can be implemented as part of the data collection. The quality and accuracy of data labeling directly influence the mode's performance, as labels serve as the definitive guide that the model uses to learn the relationships between the input features and the desired output. Effective labeling ensures that the model is trained on correct and clear examples, thus enhancing its ability to generalize from the training data to real-world scenarios.

In some instances, the ground truth values (labels) are provided within raw data. For example, when the raw data is a DNA segment, the label may be whether the variant is present in the DNA segment. The label may be based on the high confident variant calls or may be manually labeled by a trained practitioner. Labeling techniques can vary significantly depending on the type of data and the specific requirements of the project. Manual labeling, where human annotators label the data, is one method that can be used. This approach is useful when a detailed understanding and judgment are required. However, manual labeling is time-consuming and prone to inconsistency, especially with many annotators. To mitigate this, semi-automated labeling tools may be used as part of data subsystem 405 to pre-label data using algorithms, which human annotators may then review and correct as needed. Another approach is active learning, a technique where the model being developed is used to label new data iteratively. The model suggests labels for new data points, and human annotators may review and adjust certain predictions such as the most uncertain predictions. This technique optimizes the labeling effort by focusing human resources on a subset of the data, e.g., the most ambiguous cases, improving efficiency and label quality through continuous refinement.

Once collected, generated, preprocessed, and/or labeled, the data may then be split into the training and validation datasets 406. The training and validation datasets 406 may comprise the raw data and/or the preprocessed data. The training and validation datasets 406 are typically split into at least three subsets of data: training, validation, and testing. The training set is used to fit the model, where the machine learning model learns to make inferences based on the training data. The validation set, on the other hand, is utilized to tune hyperparameters 408 and prevent overfitting by providing a sandbox for model selection. Finally, the testing set serves as a new and unseen dataset for the model, used to simulate real-world application and evaluate the final model's performance. The process of splitting ensures that the model can perform well not just on the data it was trained on, but also on new, unseen data, thereby validating and testing its ability to generalize.

Various techniques can be employed to split the data effectively, with each method aiming to maintain a good representation of the overall dataset in each subset. A simple random split (e.g., a 70/20/10%, 80/10/10%, or 60/25/15%) is the most straightforward approach, where examples from the data are randomly assigned to each of the three sets. However, more sophisticated methods may be necessary to preserve the underlying distribution of data. For instance, stratified sampling may be used to ensure that each split reflects the overall distribution of a specific variable, particularly useful in cases where certain categories or outcomes are underrepresented. Another technique, k-fold cross-validation, involves rotating the validation set across different subsets of the data, maximizing the use of available data for training while still holding out portions for validation. These methods help in achieving more robust and reliable model evaluation and are useful in the development of predictive models that perform consistently across varied datasets.

Data subsystem 405 is also used for collecting, generating, setting, or implementing the hyperparameters 408 for the training and validation subsystem 415. The hyperparameters 408 control the overall behavior of the models. Unlike model parameters 445 that are learned automatically during training, the hyperparameters 408 are set before training begins and have a significant impact on the performance of the model. For example, in a neural network, hyperparameters include the learning rate, number of layers, number of neurons/nodes per layer, activation functions, convolution kernel width, the number of kernels for a model, the number of graph connections to make during a lookback period, and the maximum depth of a tree in a random forest among others. These settings can determine how quickly a model learns, its capacity to generalize from training data to unseen data, and its overall complexity. Correctly setting hyperparameters is important because inappropriate values can lead to models that underfit or overfit the data. Underfitting occurs when a model is too simple to learn the underlying pattern of the data, and overfitting happens when a model is too complex, learning the noise in the training data as if it were signal.

### (B) Training, Validation, and Testing

The training and validation subsystem 415 is comprised of a combination of specialized hardware and software to efficiently handle the computational demands required for training, validating, and testing a machine learning model. On the hardware side, high-performance GPUs (Graphics Processing Units) may be used for their ability to perform parallel processing, drastically speeding up the training of complex models, especially deep learning networks. CPUs (Central Processing Units), while generally slower for this task, may also be used for less complex model training or when parallel processing is less critical. TPUs (Tensor Processing Units), designed specifically for tensor calculations, provide another level of optimization for machine learning tasks. On the software side, a variety of frameworks and libraries are utilized, including TensorFlow, PyTorch, Keras, and scikit-learn. These tools offer comprehensive libraries and functions that facilitate the design, training, validation, and testing of a wide range of machine learning models across different computing platforms, whether local machines, cloud-based systems, or hybrid setups, enabling developers to focus more on model architecture and less on underlying computational details.

Training is the initial phase of developing machine learning models 430 where the model learns to make predictions or decisions based on training data provided from the training and validation datasets 406. During this phase, the model iteratively adjusts its model parameters 445 to achieve a preset optimization condition. In a supervised machine learning training process, the preset optimization condition can be achieved by minimizing the difference between the model output (e.g., predictions, classifications, or decisions) and the ground truth labels in the training data. In some instances, the preset optimization condition can be achieved when the preset fixed number of iterations or epochs (full passes through the training dataset) is reached. In some instances, the preset optimization condition is achieved when the performance on the validation dataset stops improving or starts to degrade. In some instances, the preset optimization condition is achieved when a convergence criterion is met, such as when the change in the model parameters falls below a certain threshold between iterations. This process, known as fitting, is fundamental because it directly influences the accuracy and effectiveness of the model.

In an exemplary training phase performed by the training and validation subsystem 415, the training subset of data is input into the machine learning algorithms 420 to find a set of model parameters 445 (e.g., weights, coefficients, trees, feature importance, and/or biases) that minimizes or maximizes an objective function (e.g., a loss function, a cost function, a contrastive loss function, a cross-entropy loss function, an Out-of-Bag (OOB) score, etc.). To train the machine learning algorithms 420 to achieve accurate predictions, “errors” (e.g., a difference between a predicted label and the ground truth label) need to be minimized. In order to minimize the errors, the model parameters can be configured to be incrementally updated by minimizing the objective function over the training phase (“optimization”). Various different techniques may be used to perform the optimization. For example, to train machine learning algorithms such as a neural network, optimization can be done using back propagation. The current error is typically propagated backwards to a previous layer, where it is used to modify the weights and bias in such a way that the error is minimized. The weights are modified using the optimization function. Other techniques such as random feedback, Direct Feedback Alignment (DFA), Indirect Feedback Alignment (IFA), Hebbian learning, and the like can also be used to update the model parameters 445 in a manner as to minimize or maximize an objective function. This cycle is repeated until a desired state (e.g., a predetermined minimum value of the objective function) is reached.

The training phase is driven by three primary components: the model architecture (which defines the structure of the machine learning algorithm(s) 420), the training data (which provides the examples from which to learn), and the learning algorithm (which dictates how the model adjusts its model parameters). The goal is for the model to capture the underlying patterns of the data without memorizing specific examples, thus enabling it to perform well on new, unseen data.

The model architecture is the specific arrangement and structure of the various components and/or layers that make up a model. In the context of a neural network, the model architecture may include the configuration of layers in the neural network, such as the number of layers, the type of layers (e.g., convolutional, recurrent, fully connected), the number of neurons in each layer, and the connections between these layers. In the context of a random forest consisting of a collection of decision trees, the model architecture may include the configuration of features used by the decision trees, the voting scheme, and hyperparameters such as the number of trees in the forest, the maximum depth of each tree, the minimum number of samples required to split a node, and the maximum number of features to consider when looking for the best split. In some instances, the model architecture is configured to perform multiple tasks. For example, a first component of the model architecture may be configured to perform a feature selection function, and a second component of the model architecture may be configured to perform a feature scoring function. The different components may correspond to different algorithms or models, and the model architecture may be an ensemble of multiple components.

Model architecture also encompasses the choice and arrangement of features and algorithms used in various models, such as decision trees or linear regression. The architecture determines how input data is processed and transformed through various computational steps to produce the output. The model architecture directly influences the model's ability to learn from the data effectively and efficiently, and it impacts how well the model performs tasks such as classification, regression, or prediction, adapting to the specific complexities and nuances of the data it is designed to handle.

The model architecture can encompass a wide range of machine learning algorithms 420 suitable for different kinds of tasks and data types. Examples of machine learning algorithms 420 include, without limitation, linear regression, logistic regression, decision tree, Support Vector Machines, Naives Bayes algorithm, Bayesian classifier, linear classifier, K-Nearest Neighbors, K-Means, random forest, dimensionality reduction algorithms, grid search algorithm, genetic algorithm, AdaBoosting algorithm, Gradient Boosting Machines, and Artificial Neural Networks such as convolutional neural network (“CNN”), an inception neural network, a U-Net, a V-Net, a residual neural network (“Resnet”), a transformer neural network, a recurrent neural network, a Generative adversarial network (GAN), or other variants of Deep Neural Networks (“DNN”) (e.g., a multi-label n-binary DNN classifier or multi-class DNN classifier). These algorithms can be implemented using various machine learning libraries and frameworks such as TensorFlow, PyTorch, Keras, and scikit-learn, which provide extensive tools and features to facilitate model building, training, validation, and testing.

The learning algorithm is the overall method or procedure used to adjust the model parameters 445 to fit the data. It dictates how the model learns from the data provided during training. This includes the steps or rules that the algorithm follows to process input data and make adjustments to the model's internal parameters (e.g., weights in neural networks) based on the output of the objective function. Examples of learning algorithms include gradient descent, backpropagation for neural networks, and splitting criteria in decision trees.

Various techniques may be employed by training and validation subsystem 415 to train machine learning models 430 using the learning algorithm, depending on the type of model and the specific task. For supervised learning models, where the training data includes both inputs and expected outputs (e.g., ground truth labels), gradient descent is a possible method. This technique iteratively adjusts the model parameters 445 to minimize or maximize an objective function (e.g., a loss function, a cost function, a contrastive loss function, etc.). The objective function is a method to measure how well the model's predictions match the actual labels or outcomes in the training data. It quantifies the error between predicted values and true values and presents this error as a single real number. The goal of training is to minimize this error, indicating that the model's predictions are, on average, close to the true data. Common examples of loss functions include mean squared error for regression tasks and cross-entropy loss for classification tasks.

The adjustment of the model parameters 445 is performed by the optimization function or algorithm, which refers to the specific method used to minimize (or maximize) the objective function. The optimization function is the engine behind the learning algorithm, guiding how the model parameters 445 are adjusted during training. It determines the strategy to use when searching for the best weights that minimize (or maximize) the objective function. Gradient descent is a primary example of an optimization algorithm, including its variants like stochastic gradient descent (SGD), mini-batch gradient descent, and advanced versions like Adam or RMSprop, which provide different ways to adjust learning rates or take advantage of the momentum of changes. For example, in training a neural network, backpropagation may be used with gradient descent to update the weights of the network based on the error rate obtained in the previous epoch (cycle through the full training dataset). Another technique in supervised learning is the use of decision trees, where a tree-like model of decisions is built by splitting the training dataset into subsets based on an attribute value test. This process is repeated on each derived subset in a recursive manner called recursive partitioning.

In unsupervised learning, where training data does not include labels, different techniques are used. Clustering is one method where data is grouped into clusters that maximize the similarities of data within the same cluster and maximize the differences with data in other clusters. The K-Means algorithm, for example, assigns each data point to the nearest cluster by minimizing the sum of distances between data points and their respective cluster centroids. Another technique, Principal Component Analysis (PCA), involves reducing the dimensionality of data by transforming it into a new set of variables, the principal components, which are uncorrelated and ordered so that the first few retain most of the variation present in all of the original variables. These techniques help uncover hidden structures or patterns in the data, which can be essential for feature reduction, anomaly detection, or preparing data for further supervised learning tasks.

Validating is another phase of developing machine learning models 430 where the model is checked for deficiencies in performance and the hyperparameters 408 are optimized based on validation data provided from the training and validation datasets 406. The validation data helps to evaluate the model's performance, such as accuracy, precision, recall, or F1-score, to gauge how well the training is ongoing, for example, by monitoring if an underfitting or overfitting is occurring. Hyperparameter optimization, on the other hand, involves adjusting the settings that govern the model's learning process (e.g., learning rate, number of layers, size of the layers in neural networks) to find the combination that yields the best performance on the validation data. One optimization technique is grid search, where a set of predefined hyperparameter values are systematically evaluated. The model is trained with each combination of these values, and the combination that produces the best performance on the validation set is chosen. Although thorough, grid search can be computationally expensive and impractical when the hyperparameter space is large. A more efficient alternative optimization technique is random search, which samples hyperparameter combinations from a defined distribution randomly. This approach can in some instances find a good combination of hyperparameter values faster than grid search. Advanced methods like Bayesian optimization, genetic algorithms, and gradient-based optimization may also be used to find optimal hyperparameters more effectively. These techniques model the hyperparameter space and use statistical methods to intelligently explore the space, seeking hyperparameters that yield improvements in model performance.

An exemplary validation process includes iterative operations of inputting the validation subset of data into the trained algorithm(s) using a validation technique such as K-Fold Cross-Validation, Leave-one-out Cross-Validation, Leave-one-group-out Cross-Validation, Nested Cross-Validation, or the like, to fine-tune the hyperparameters and ultimately find the optimal set of hyperparameters. In some instances, a 5-fold cross-validation technique may be used to avoid overfitting the trained algorithm and/or to limit the number of selected features per split to the square-root of the total number of input features. In some instances, training dataset is split into 5 equal-size cohorts (or about equal-size), and every four of the cohorts are used to train an algorithm to generate five models (e.g., cohorts #1, 2, 3, and 4 are used to train and generate model 1, cohorts #1, 2, 3, and 5 are used to train and generate model 2, cohorts #1, 2, 4, and 5 are used to train and generate model 3, cohorts #1, 3, 4, and 5 are used to train and generate model 4, and cohorts #2, 3, 4 and 5 are used to train and generate model 5). Each model is evaluated (or validated) using the unused cohort in the training (e.g., for model 5, cohort #1 is used for validation). The overall performance of the training can be evaluated by an average performance of the five models. K-fold cross-validation provides a more robust estimate of a model's performance compared to a single training/validation split because it utilizes the entire dataset for both training and evaluation and reduces the variance in the performance estimate.

Once a machine learning model has been trained and validated, it undergoes a final evaluation using test data provided from the training and validation datasets 406, which is a separate subset of the data that has not been used during the training or validation phases. This step is crucial as it provides an unbiased assessment of the model's performance in simulating real-world operation. The test dataset serves as new, unseen data for the model, mimicking how the model would perform when deployed in actual use. During testing, the model's predictions are compared against the true values in the test dataset using various performance metrics such as accuracy, precision, recall, F1, AUC, and mean squared error, depending on the nature of the problem (classification or regression). This process helps to verify the generalizability of the model-its ability to perform well across different data samples and environments-highlighting potential issues like overfitting or underfitting and ensuring that the model is robust and reliable for practical applications. The machine learning models 430 are fully validated and tested once the output predictions have been deemed acceptable by user defined acceptance parameters. Acceptance parameters may be determined using correlation techniques such as Bland-Altman method and the Spearman's rank correlation coefficients and calculating performance metrics such as the error, accuracy, precision, recall, receiver operating characteristic curve (ROC), etc.

### (C) Inference Phase for Machine Learning Models

The inference subsystem 425 is comprised of various components for deploying the machine learning models 430 in a production environment (e.g., use in a genetic assay as described with respect to FIG. 2). Deploying the machine learning models 430 includes moving the models from a development environment (e.g., the training and validation subsystem 415, where it has been trained, validated, and tested), into a production environment where it can make inferences on real-world data (e.g., input data 450). This step typically starts with the model being saved after training, including its parameters and configuration such as final architecture and hyperparameters. It is then converted, if necessary, into a format that is suitable for deployment, depending on the deployment environment. For instance, a model trained in a scientific computing environment such as Python might be converted into a Java-friendly format for integration into a larger enterprise application. Deployment can be conducted on various platforms, including on-premises servers, cloud environments like AWS, Azure, Google.

Once deployed, the model is ready to receive input data 450 and return outputs (e.g., inferences 455). In some instances, the model resides as a component of a larger system or service (e.g., including additional downstream applications 435). In some instances, the machine learning models 430 and/or the inferences 455 can be used by the downstream applications 435 to provide further information. For example, the inferences 455 can be used to aid qualified personnel (e.g., oncologists) to help diagnosis and/or determine whether treatment should be administered to a patient. In some instances, the inferences 455 can be used to aid qualified personnel to determine a specific type of treatment to administer to a patient based on the inference results. The downstream applications can be configured to generate an output 460. In some instances, the output 460 comprises a report including inferences 455 and information generated by the downstream applications 435.

In an exemplary inference subsystem 425, the input data 450 includes sequencing data generated from one or more biological samples from a patient having been diagnosed a disease (e.g., cancer) or suspect to have or will develop the disease. The sequencing data may be generated by performing NGS on nucleic acid obtained from the one or more biological samples collected from the patient. The one or more biological samples may be a single or multiple tissue samples (e.g., a bladder tissue, lung tissue, tumor section, etc.) or liquid sample obtained from the patient.

To manage and maintain its performance, a deployed model may be continuously monitored to ensure it performs as expected over time. This involves tracking the model's prediction accuracy, response times, and other operational metrics. Additionally, the model may require retraining or updates based on new data or changing conditions in the environment it is applied in. This can be useful because machine learning models can drift over time due to changes in the underlying data they are making predictions on—a phenomenon known as model drift. Therefore, maintaining a machine learning model in a production environment often involves setting up mechanisms for performance monitoring, regular evaluations against new test data, and potentially periodic updates and retraining of the model to ensure it remains effective and accurate in making predictions.

FIGS. 5A-5D are block diagrams illustrating a process 500 for training, validating, and testing a Sanger bypass assay platform or system in accordance with various embodiments. The goal of process 500 is to generate a Sanger bypass assay platform or system capable of making predictions on the statistical likelihood of a variant being a true positive based on a set of measurable quality features extracted from processed sequencing data (e.g., annotated VCF files). In other words, the model is trained to have a high false positive “capture rate” (sensitivity: TP/(TP+FN) in the machine learning context), minimizing the number of false positive variant calls that could be missed by the model and allowed onto a final patient report. The model should also have a low true positive flagging rate (“TP flag rate,” false positive rate: FP/(TN+FP) in the machine learning context, true calls incorrectly marked as false), meaning that the fewest true positive variant calls will be flagged by the model to be sent for confirmatory testing. This goal is achieved using three dependent model development phases: a leave-one-out cross-validation (LOOCV) phase 505 (FIG. 5A), a second training and testing phase 510 (FIGS. 5B and 5C) that executes multiple rounds of training and testing to optimize machine learning models, and a final model validation phase 515 (FIG. 5D). Although not explicitly discussed, it should be understood that the false positive capture rate and/or true positive flag rate may be modified based on the type of sample. For example, to ensure capture of clinically relevant variants that may fall outside of the reporting criteria, the capture rate may be increased.

As depicted in FIG. 5A, the LOOCV phase 505 is for implementing a cross-validation approach to evaluate how well different machine learning models perform at capturing false positive variants and not flagging true positive variants across different genetic backgrounds. This method uses different portions of the data to train and test machine learning algorithms on different iterations. Given S samples, machine learning algorithms are trained on (S-1) samples then evaluated using a left-out sample to simulate receiving a “new” sample. This is performed a total of S times (e.g., each sample is left out once), leading to a multifold LOOCV. The first round of the second training and testing phase 510 (see FIG. 5B) is for a classical validation approach that uses a single split (e.g., 50/50%) between portions of the data and scaling all the quality features in the first 50% of the data to create a training set used to further train the machine learning algorithms. The second 50% of the data is a validation/test set used to further validate the machine learning algorithms on data that the machine learning algorithms have never seen before. The goal of the first round of the second training and testing phase 510 is to determine the relative contribution of each quality feature associated with either true or false positive variants and generate improved machine learning models to be implemented in the second round of the second training and testing phase 510 (see FIG. 5C). The second round also uses a classical validation approach where the first 50% of the data is used for training and the second 50% is used for testing. The first 50% of the data has oversampling techniques applied and is used to train the improved machine learning models from the first round of the second phase. The goal of the second round of the second training and testing phase 510 (FIG. 5C) is to determine if oversampling improves the performance of the machine learning models and to output final machine learning models to be implemented in a Sanger bypass assay platform or system. The final model validation phase 515 (see FIG. 5D), is for validating the selected optimized machine learning models in the Sanger bypass assay platform or system. To achieve this, all the data initially used to train/validate/test the machine learning models is input into the Sanger bypass assay platform or system and the output predictions of the variants are assessed by comparing the predicted output to ground truths. The combination of training and testing in the LOOCV phase 505 (FIG. 5A), the multiple different rounds of the second training and testing phase 510 (FIGS. 5B and 5C), and the final model validation phase 515 (FIG. 5) is used to generate final machine learning models that are implemented into the Sanger bypass assay platform or system that are capable of making predictions on the statistical likelihood of a variant being a true positive.

Initially in FIG. 5A, a labeled variant dataset 517 comprised of truth labeled variants and their quality features are accessed. For example, the labeled variant dataset 517 may be stored in a database or memory storage device and accessed by a training system such as the training and validation subsystem 415 described with respect to FIG. 4. Further, the labeled variant dataset 517 can comprise variant calls from multiple different genetic backgrounds (e.g., multiple cell lines' exomes). The variant calls in the labeled variant dataset 517 should be high-confidence variant calls. In other words, the variant calls should be either confirmed or benchmarked in-house using various sequencing and variant calling technologies and/or obtained from a reliable source known for distributing or providing confirmed or benchmarked variant call data. The labeled variant dataset 517 comprise sets of measurable quality features that are used for training and testing various machine learning algorithms. The quality features include but are not necessarily limited to the following: read frequency, read count metrics, coverage, quality, read position probability, read direction probability, homopolymer traits, and overlap with low-complexity sequence (see Table 1).

TABLE 1
Complete list of quality features selected for training.

| Features/Variables | Definition |
|---|---|
| Read count | The number of ‘countable’ reads supporting the allele. Countable |
| | reads are defined based on the user-defined settings in the variant |
| | callers. |
| Coverage | The read coverage at this position. Only ‘countable’ reads are |
| | considered. |
| Frequency | ‘Count’ divided by ‘Coverage’ |
| Forward count | The number of ‘countable’ forward reads supporting the allele |
| Reverse count | The number of ‘countable’ reverse reads supporting the allele |
| Forward/Reverse ratio | The number of ‘countable’ forward reads divided by the number of |
| | ‘countable’ reverse |
| Average quality | The average base quality score of the bases supporting a variant. |
| Probability | posterior probability of the particular site-type called. |
| Read position probability | The test probability for the test of whether the distribution of the |
| | read positions variant in the variant carrying reads is different |
| | from that of all the reads covering the variant position. |
| Read direction probability | The test probability for the test of whether the distribution among |
| | forward and reverse reads of the variant carrying reads is different |
| | from that of all the reads covering the variant position |
| Homopolymer | The column contains “Yes” if the variant is likely to be a |
| | homopolymer error and “No” if not. This is assessed by inspecting |
| | all variants in homopolymeric regions longer than 2. A variant |
| | will get the mark “yes” if it is a homopolymeric length variation |
| | of the reference allele, or a length variation of another variant that |
| | is a homopolymeric variation of the reference allele. |
| Homopolymer length | homopolymer length |
| Complex region | region with known homology and/or low mappability |

As described herein, machine learning algorithms or machine learning models can include any machine learning algorithms or machine learning models known to one skilled in the art. By way of example, machine learning algorithms or machine learning models can include Logistic Regression, Random Forest, EasyEnsemble, AdaBoost, and Gradient Boosting.

To facilitate model training and testing in the LOOCV phase 505 (FIG. 5A) and the multiple rounds of the second training and testing phase 510 (FIGS. 5B and 5C), the labeled variant dataset 517 is split into a first subset of training data 520 and a second subset of testing data 525 (illustrated in FIGS. 5A-5D). The splitting may be performed based on a predefined ratio (e.g., a 50/50%, 90/10%, or 70/30%) or the splitting may be performed randomly or in accordance with a more complex validation technique such as K-Fold Cross-Validation, Leave-one-out Cross-Validation, Leave-one-group-out Cross-Validation, Nested Cross-Validation, or the like to minimize sampling bias and overfitting. As illustrated in FIG. 5A, the first subset of training data 520 comprises a number of examples (S) that comprise labeled variant calls and annotated quality features for training and testing machine learning algorithms. The second subset of testing data 525 comprises a number of examples (T) that comprise labeled variant calls and annotated quality features for testing machine learning algorithms. The variant calls may be further annotated (e.g., in the instance the variant call is associated with an example in a training subset) with a ground truth label to allow the machine learning algorithms to learn which features are the most significant predictors of the presence or absence of a variant. Since false positive variant calls (variants called by a bioinformatics pipeline but absent from the truth set) are the primary target, false positive variant calls are labeled as positives (e.g., binary label “1”) when passed to the machine learning algorithms. Similarly, true positive variant calls passed to the machine learning algorithms are labeled as negatives (e.g., binary label “0”).

The LOOCV phase 505 (FIG. 5A) splits the first subset of training data 520 into a cross-validation training set 530 (a-n) and a cross-validation testing set 535 (a-n), using all but one example (S−1) as part of the training set. The machine learning algorithms 540 are then trained on the cross-validation training set 530 (a), using all quality features to generate partially trained machine learning algorithms 545. The partially trained machine learning algorithms 545 are then used to evaluate the consistency of the false positive capture rates and true positive flagging rates of the partially trained machine learning algorithms 545 across different genetic backgrounds. This training/testing process is repeated (S) times for each variant type being examined (where (S) is the total number of examples in the first subset of training data 520), leaving out a different observation from the cross-validation training set 530 (a-n) each time for the cross-validation testing set 535 (a-n) generating cross-validated machine learning algorithms 550 that are used in the first round of the second training and testing phase 510 (FIG. 5B). The training of machine learning algorithms 540 using the cross-validation training set 530 (a-n) and the testing of partially trained machine learning algorithms 545 using the cross-validation testing set 535 (a-n) may be performed in a similar manner as described with respect to the training and validation subsystem 415 described with respect to FIG. 4.

The cross-validated machine learning algorithms 550 are used in the first round of the second training and testing phase 510 of process 500 (FIG. 5B). The cross-validated machine learning algorithms 550 are trained again using the first subset of training data 520. However, prior to training, the first subset of training data 520, is scaled 552 so that all quality features contributing to the machine learning algorithms are on a relatively similar scale close to normal distribution. This prevents quality features with different units (e.g., seconds, minutes, hours) and thus different scales from greatly biasing the model's weight values. Methods for scaling 552 can include: MinMaxScaler, RobustScaler, StandardScaler, Normalizer, and any other methods known to one of skill in the art. Another benefit of scaling 552 the first subset of training data 520 is to identify high-impact quality features and to remove redundant features, which can improve the overall performance of the cross-validated machine learning algorithms 550. High-impact quality features (also referred to as a subset of high-impact quality features or a limited subset of quality features) refer to the quality features (listed in Table 1) that contribute the most to either true positive or false positive variant detection. Based on the coefficient values or the importance values for each quality feature generated from training, high-impact quality features are selected for each cross-validated machine learning algorithms 550. In some cases, scaling 552 does not have an effect on the performance of the cross-validated machine learning algorithms 550, and those cross-validated machine learning algorithms 550 are retrained using an unscaled version of the first subset of training data 520 with all quality features. In other instances, scaling 552 does have an effect on the performance of the cross-validated machine learning algorithms 550 and those models will use only their associated high-impact quality features. Training on the scaled or unscaled version of the first subset of training data 520 generates post-trained machine learning algorithms 555 that are then tested, using the second subset of testing data 525. Testing confirms that the post-trained machine learning algorithms 555 trained on high-impact quality features or trained on all the quality features do in fact show improved performance. At the end of testing, improved machine learning algorithms 560 are generated and used in the second round of the second training and testing phase 510 (FIG. 5C).

During the second round of the second training and testing phase 510 (FIG. 5C), the first subset of training data 520 and the second subset of testing data 525 are again used to train and test the improved machine learning algorithms. Further, the second round comprises applying oversampling techniques to the variant calls in the in the first subset of training data 520. Oversampling 554 involves enriching the minority class of data points as a way to compensate for imbalanced data (e.g., NGS analysis identifies a disproportionately larger number of true positive variants compared to false positive variants). Examples of this approach include randomly duplicating data points from the minority dataset (false positive variants) like in simple over sampling (SOS) or generating synthetic data points according to a k-nearest neighbor analysis of minority data point clustering as in synthetic minority oversampling (SMOTE). The improved machine learning algorithms 560 are trained on a balanced version (e.g., oversampling applied) of the first subset of training data 520 to assess if balanced data improves models' performance (e.g., the false positive capture rates and true positive flagging rates). In some instances, training on a balanced dataset does not improve the performance of some of the improved machine learning models, in which case, they are retrained on the first subset of training data 520 without oversampling (e.g., imbalanced dataset). Training generates optimized machine learning algorithms 565 that are then tested using the second subset of testing data 525. Testing confirms that the optimized machine learning algorithms 565 trained on either balanced data or imbalanced data do in fact show improved performance. At the end of testing, final machine learning models 570 are generated and used in the final model validation phase 515 (FIG. 5D). The final machine learning models 570 are trained and validated on any combination of high-impact quality features/all the quality features and balance/imbalanced data. For example, one of the final machine learning models 570 may be trained on the high-impact quality features and balanced data. As another example, another final machine learning model 570 may be trained on all the quality features and the imbalanced data. Once the second round of the second training and testing phase 510 is complete, one or more of the final machine learning models 570 with the best overall false positive capture rate and true positive flagging rate are selected and input into the first tier or second tier of the Sanger bypass assay platform or system for a final validation in the final model validation phase 515 (FIG. 5D). Although not explicitly described, it should be understood that the second training and testing phase 510 may have additional rounds of training and testing (e.g., more than two) when necessary.

The final model validation phase 515 (FIG. 5D) uses the entire labeled variant dataset 517 to evaluate how the selected one or more final machine learning models 570 perform in the Sanger bypass assay platform or system. The final machine learning models 570 can be used to predict a response value (e.g., statistical likelihood of a variant being a true positive or false positive) for the variants in the labeled variant dataset 517 and a measure of how well the model performed or made the prediction of the response value is calculated (e.g., mean squared error (MSE)). After completion of the LOOCV phase 505 (FIG. 5A), the multiple rounds of the second training and testing phase 510 (FIGS. 5B and 5C), and the final model validation phase 515 (FIG. 5D), the final machine learning models 570 are implemented in the Sanger bypass assay platform or system. In addition, the final machine learning models 570 report out a list of variants that pass through a Sanger bypass decision tree (described in detail in FIG. 5) that determines which variants will either require Sanger sequencing confirmation or which variants qualify to be bypassed for Sanger sequencing confirmation.

### V. Additional Flowcharts

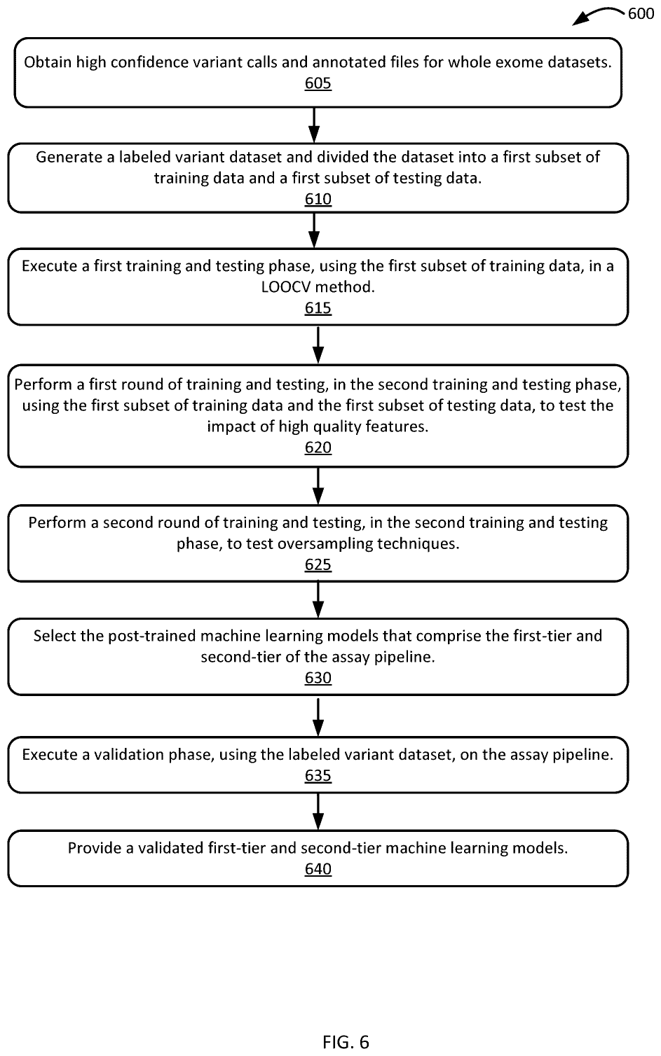

FIG. 6 is a flowchart illustrating a process 600 for training one or more machine learning models to classify variants as either being false positives or true positives based on quality features in accordance with various embodiments. The processing depicted in FIG. 6 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 6 and described below is intended to be illustrative and non-limiting. Although FIG. 6 depicts the various processing steps occurring in a particular sequence or order, this is not intended to be limiting. In certain alternative embodiments, the steps may be performed in some different orders, or some steps may also be performed in parallel.

At block 605, high-confidence variant calls labelled as truths and annotated files generated from whole exome datasets are obtained (e.g., accessed from a sequencer or database). The high-confidence variant calls can be accessed from a publicly available data source (e.g., GIAB benchmark VCF files) and have been validated using one or more sequencing technologies (Sanger, NGS, and the like). Further, the high-confidence variants comprise small variants and variants in more difficult regions of the genome. The annotated files may be generated as part of performing a whole exome sequencing assay. In addition, the annotated files comprise one or more variants and their quality features identified from the whole exome sequencing assays.

At block 610, a labelled variant dataset is generated by annotating the one or more variant calls in the annotated files with a truth label based on the truth labels from the high-confidence variant dataset obtained in block 605. Once annotation is complete, the labelled variant dataset comprises variants with true positive labels and false positive labels. A true positive label indicates the variant was found in both the high-confidence variant dataset and the annotated files, whereas a false positive label indicates the variant was not found in the high-confidence variant dataset but was found in the annotated files. Finally, the labelled variant dataset is divided (e.g., 50/50%) into a first subset of training data and a first subset of testing data using stratification of the truth label. In so doing, the first subset of training data and the first subset of testing data comprise similar proportions of true positive and false positive variants.

At block 615, the first subset of training data is used in a first training and testing phase that applies a LOOCV method to train and validate one or more machine learning algorithms. The purpose of the first training and testing phase is to evaluate the consistency of the one or more machine learning models to generate high false positive capture rates and low true positive flagging rates across different genomic backgrounds. Initially, the first subset of training data is split into a cross-validation training dataset and a cross-validation testing dataset. The cross-validation training dataset comprises one less of the total number of samples (S−1), while the cross-validation testing dataset comprises the left-out sample. For example, if there are a total of 7 samples in the first subset of data, the cross-validation training dataset may comprise samples 1, 2, 3, 4, 5, and 6 while the cross-validation testing dataset comprises sample 7. The total number of times the first subset of data is split is based on the total number of samples, where each sample is left out once, allowing for multiple iterations of the LOOCV phase. During the training, the one or more machine learning algorithms use the cross-validation training dataset and all quality features to generate initial false positive capture rates and true positive flagging rates for the one or more partially trained machine learning algorithms. The partially trained machine learning algorithms are then tested/validated, using the cross-validation testing dataset, to assess how consistently the partially trained machine learning models perform across different genetic backgrounds. At the end of testing, one or more cross-validated machine learning models are generated and input into the second phase of training and testing.

At block 620, the one or more cross-validated machine learning algorithms enter a second training and testing phase where one or more rounds of training and testing are performed using the first subset of training data (used in block 615) and the first subset of testing data (generated in block 610). During a first round of the one or more rounds of the second training and testing phase, the quality features comprising the first subset of training data are scaled to generate a scaled subset of training data. Scaling prevents quality features with different units (e.g., seconds, minutes, hours) from greatly biasing the model's weight values and helps to remove quality features with similar model contributions. The one or more cross-validated machine learning algorithms are trained on the scaled subset of training data to generate one or more post-trained machine learning models. During training, the coefficient values or importance values of the quality features are evaluated for each of the one or more post-trained machine learning algorithms to identify high-impact quality features that contribute the most to the associated true positive or false positive variant label. The high-impact quality features are selected from the list of quality features displayed in Table 1 and do not have to be the same for all the post-trained machine learning algorithms. In some cases, training on the scaled dataset does not influence the coefficient values or the importance values. Accordingly, those post-trained machine learning models are retrained using the first subset of training data without any scaling and all the quality features are used. The one or more post-trained machine learning models trained on the high-impact quality features and the one or more post-trained machine learning models trained on all the quality features are then tested using the first subset of testing data to validate that training on the high-impact quality features or all the quality features improves the false positive capture rate and the true positive flagging rate of the one or more optimized machine learning models. Following testing, one or more improved machine learning models are generated that are trained on either: (i) high-impact quality features or (ii) all the quality features.

At block 625, the second round of the one or more rounds of the second training and testing phase is executed. During the second round, the first subset of training data undergoes SOS or SMOTE oversampling to generate a balanced dataset. Oversampling increases the proportion of false positive variants to true positive variants in the first subset of training data, essentially making the data more “balanced”. The improved machine learning algorithms from block 620 (e.g., the one or more improved machine learning models trained on high-impact quality features and the one or more improved machine learning models trained on all quality features) are trained on the balanced dataset to generate one or more optimized machine learning models. Like in the first round, training in the second round also comprises fine tuning a set of parameters for the one or more improved machine learning models trained on the high-impact quality features and the one or more improved machine learning models trained on all the quality features that maximizes the false positive capture rate and minimizes the true positive flagging rate so that a value of the loss or error function using the set of parameters is smaller than a value of the loss or error function using another set of parameters in a previous iteration. Next, the false positive capture rates and the true positive flagging rates are evaluated to determine whether the balanced dataset improves the performance of the one or more optimized machine learning models. In some instances, training on the balanced data does improve the performance of the one or more optimized machine trained on the high-impact quality features/all the quality features. Other times, training on the balanced data does not improve the performance of the one or more optimized machine trained on the high-impact quality features/all the quality features. For the optimized machine learning models that did not show improved performance, they are retrained using the first subset of training data without any oversampling (e.g., imbalanced data) to achieve improved performance. Once all optimized machine learning models are generated, another round of testing is performed. Testing is done using the first subset of testing data to generate one or more final machine learning models and to validate that either training on the balanced dataset or the imbalanced dataset improves the performance of the one or more optimized machine learning models. As a result, the second round of training and testing generates one or more final machine learning models trained on (i) all quality the features and the imbalanced data, (ii) all the quality features and the balanced data, (iii) the subset of high impact quality features and the imbalanced data, and (iv) the subset of high impact quality features and the balanced data.

At block 630, several (e.g., at least three) of the one or more final machine learning models output at block 625 are selected to be implemented in the first tier and second tier of the Sanger bypass assay platform or system, based on their overall false positive capture rates and true positive flag rates. The first-tier machine learning models can include a logistic regression model trained on the high-impact quality features and SOS balanced data and a random forest classifier model trained on all quality features and imbalanced data. The second-tier machine learning model can include a gradient boosting model trained on all quality features and imbalanced data.

At block 635, a final validation, using the labeled variant dataset from block 610 is performed on the selected first- and second-tier machine learning models. Final validation involves inputting the labeled variant dataset into the Sanger bypass assay platform or system and evaluating the final output against ground truths from the high-confidence variant dataset.

At block 645, the validated first-tier and second-tier machine learning models are provided and are implemented in the Sanger bypass assay platform. The final models are trained to predict if one or more variants are true positives or false positives based on their associated quality features.

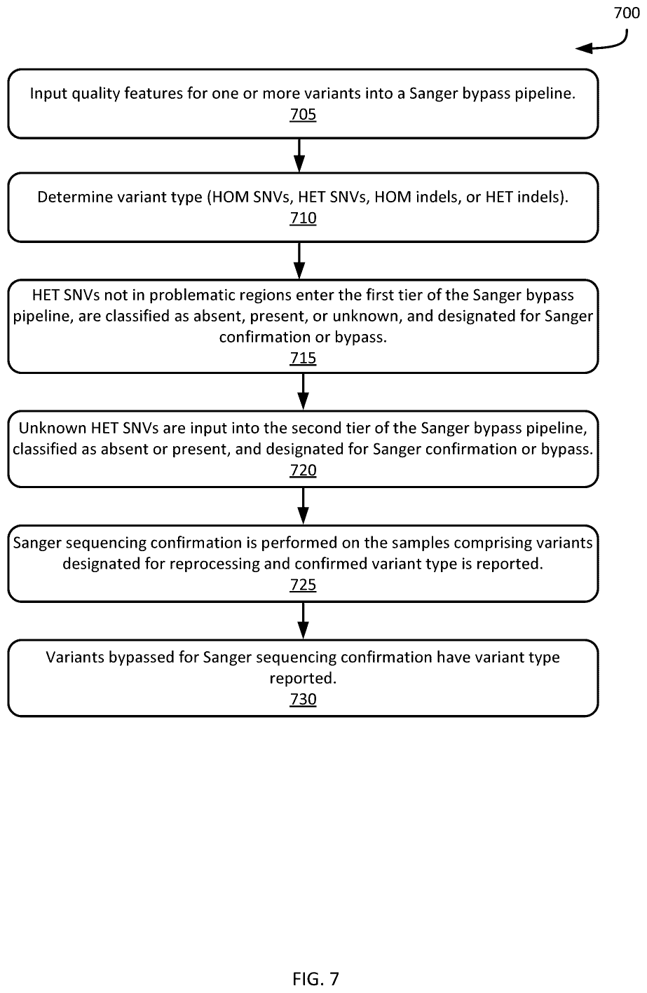

FIG. 7 is a flowchart illustrating a process 700 for how the Sanger bypass assay platform or system uses machine learning models to determine which variants will be bypassed for Sanger sequencing confirmation and which variants will require Sanger sequencing confirmation in accordance with various embodiments. The processing depicted in FIG. 7 may be implemented in software (e.g., code, instructions, program) executed by one or more processing units (e.g., processors, cores) of the respective systems, hardware, or combinations thereof (e.g., the intelligent selection machine). The software may be stored on a non-transitory store medium (e.g., on a memory device). The method presented in FIG. 7 and described below is intended to be illustrative and non-limiting. Although FIG. 7 depicts the various processing steps occurring in a particular sequence or order, this is not intended to be limiting. In certain alternative embodiments, the steps may be performed in some different orders, or some steps may also be performed in parallel.

Process 700 begins at block 705 where an annotated file comprising quality feature data for one or more variants are input into a Sanger bypass assay platform or system. The annotated file is generated from whole exome sequencing assays conducted on patient samples undergoing a clinical genetic screen. Genetic screening typically uses WES to detect one or more variants or alterations to the DNA sequence not found in the reference sequence, and then based on the clinical effect of the variant, (e.g., benign, likely benign, variant of unknown significant, likely pathogenic or pathogenic), provide next steps for patient care. Variants are often described based on their one or more nucleotides or chromosomal regions affected. Common examples include heterozygous (HET) single nucleotide variants (SNVs), homozygous (HOM) SNVs, HOM insertion-deletions (indels), or HET indels.

In some instances, the annotated file comprises WES data for clinical samples (e.g., specimens and cell lines) that are processed on several different sequencing flow cells for variant identification. Quality control steps, including filtering of variants that do not meet specific criteria or thresholds (e.g., display gene overlap, lack a GE score, come from a depleted specimen, or are known truths) are performed.

At block 710, variant type is determined (e.g., HOM SNVs, HET SNVs, HOM indel, or HET indel) and based on the variant type, the Sanger bypass pipeline makes several decisions to determine if Sanger confirmation is required. Homozygous variants (e.g., HOM SNVs and HOM indels) are almost always designated for Sanger sequencing confirmation, unless otherwise specified by a trained lab professional. HET indel variants are bypassed for Sanger confirmation if they appear in an exemption list comprising variants that are in concordance with previous data and display quality thresholds (e.g., allele frequency ranges and read coverage) consistent with heterozygous calls. See Table 10 for a list of exempt indels. Otherwise, HET indels are also designated for Sanger sequencing confirmation. HET SNVs that are found in problematic regions (e.g., areas with homology, low complexity, and repeat expansions) are also designated for Sanger sequencing confirmation.

At block 715, variants classified as HET SNVs and not located in problematic regions enter the first tier of the Sanger bypass pipeline. Here, the HET SNVs are input into at least two trained machine learning models that predict if the HET SNVs are absent (false positives), present (true positives), or unknown (could not be classified as false positive or a true positive). The trained machine learning models include a logistic regression model that uses limited high-impact quality features and data balanced via oversampling techniques (e.g., SOS) and has a confidence threshold set to greater than or equal to 0.99. In addition, the first tier also includes a random forest classifier model that uses all quality features and imbalanced data with a confidence threshold set to 0.9. See FIG. 2 for a more detailed description. HET SNVs classified as absent are designated for Sanger sequencing confirmation. HET SNVs classified as present are confirmed to pass quality thresholds (e.g., have an allele frequency between 36-65 and read coverage greater than or equal to 30). Those HET SNVs that meet the criteria and quality thresholds of the first-tier machine learning models qualify for Sanger sequencing bypass while the HET SNVs that do not meet the criteria and quality thresholds are designated for Sanger confirmation.

At block 720, the HET SNVs classified as unknown enter the second tier of the Sanger bypass pipeline. Similar to the present HET SNVs, the unknowns are confirmed to meet the quality thresholds of the first-tier machine learning models (e.g., have an allele frequency between 36-65 and read coverage greater than or equal to 30). If the unknown variant does not meet this quality threshold, it is designated for Sanger sequencing confirmation. For those that do meet the quality threshold, they are input into a third trained machine learning model. The third trained machine learning model is a gradient boosting model that uses all quality features and imbalanced data to predict if the unknown variant is absent or present. See FIG. 2 for a more detailed description. Unknowns classified as absent are designated for Sanger sequencing confirmation, while those classified as present qualify for Sanger sequencing bypass.

At block 725, Sanger sequencing confirmation is performed on the samples comprising variants designated for reprocessing. After reprocessing, the Sanger confirmed results of the variants are reported along with a notation that reprocessing was required.

At block 730, those variants designated to be bypassed for Sanger sequencing confirmation have their variant type from the original WES assay (from block 705) reported and are noted as not requiring Sanger sequencing confirmation. Once all variants have been processed and accurately reported, a final patient report is generated and provided to clinicians. The patient report can contain information pertaining to the type of genetic screen received (e.g., catalog-based carrier screen, full gene panel screen, and the like), the genomic location of the variant, variant type, nucleotide or chromosomal alteration detected, if Sanger resequencing was required, as well as any other information obtained from the Sanger bypass assay platform or system.

### VI. Examples

The following examples are offered by way of illustration, and not by way of limitation.

### Materials

Whole exome libraries for seven Genomes in A Bottle (GIAB) cell lines (Table 2) were sequenced twice on two flow cells (CBI-435 and CBI-440). The sequenced data were analyzed with the CLCBio Clinical Lab Service to generate annotated files with quality features that were used for training and testing various machine-learning algorithms. Samples included in the GIAB reference cell lines comprised a UTAH CEPH female characterized in the Hapmap Project, and two trios enrolled in the Personal Genome Project (Table 2). In addition, GIAB benchmark files containing high-confidence variant calls were also downloaded from the National Center for Biotechnology Information (NCBI) site and used as the truth set for supervised learning and model performance assessment.

TABLE 2
GIAB Reference Materials/Cell Lines Used in Validation

| NIST ID | Corriell ID | HeatMap Ethnicity | Family Association |
|---|---|---|---|
| HG001 | NA12878 | Utah/Mormon | daughter |
| HG002 | NA24385 | Ashkenazi Jewish | son |
| HG003 | NA24149 | Ashkenazi Jewish | father |
| HG004 | NA24143 | Ashkenazi Jewish | mother |
| HG005 | NA24631 | Han Chinese | son |
| HG006 | NA24694 | Han Chinese | father |
| HG007 | NA24695 | Han Chinese | mother |

### Overview of the Training and Testing Strategy

There are two primary goals of the Sanger bypass assay platform or system: (i) reduce the number of true positive variant calls being unnecessarily confirmed by Sanger sequencing, and (ii) increase the “capture rate” of false positive variants so very few if any are missed by the Sanger bypass assay platform or system and included on the final patient report. In order to design machine learning models that can enrich false positive variant calls, five different machine learning models were trained, and their performance assessed using a number of quality features.

Logistic regression, random forest, EasyEnsemble, AdaBoost, and gradient boosting machine learning models were selected for predictive modeling of high-confidence variants detected in the GIAB specimen cell lines. The features used for model training and assessment included allele frequency, read count metrics, coverage, quality, read position probability, read direction probability, homopolymer traits, and overlap with low-complexity sequence (e.g., complex regions). Also see Table 1. A labeled variant dataset was generated by annotating each variant in the GIAB cell line samples with truth labels based on the high-confidence variant calls in the GIAB benchmark file. This approach allowed the machine learning algorithms to learn which quality features were the most significant predictors of the presence or absence of a variant (also described in FIGS. 1-5). The labeled variant dataset was then split, based on truth stratification, into training and testing datasets.

A LOOCV was performed using the training dataset of the labeled variant labeled datasets (method also described in FIG. 2) and two minimum acceptable capture rates of 95% and 99% were tested. A capture rate of 95% or 99% indicates that 5/100 or 1/100 false positive calls are missed, respectively. Given S samples, the five machine learning models were first trained on (S−1) samples, then tested using the left-out sample to simulate receiving a “new” sample. Because false positive variant calls (variants called by the pipeline but absent from the truth set) are the primary target, they were labeled as positives (binary label “1”) when passed to the machine-learning algorithms. Similarly, true positive variant calls passed to the machine-learning algorithm were labeled as negatives (binary label “0”). LOOCV was performed a total of S times (each sample was left out once), leading to a sevenfold LOOCV analysis. In addition, the process was repeated for each of the four types of variants: heterozygous (HET) SNV, homozygous (HOM) SNV, HET indels, and HOM indels. In total, the LOOCV process produced 280 tests to account for all 7 GIAB cell lines, the 5 machine learning models, the 2 different recall rates assessed, and the 4 variant types tested.

After completion of the LOOCV, a second training and testing was performed, using both the training and testing datasets from the labeled variant datasets. The second training and testing comprised of two rounds: a first round for identifying high impact quality features and a second round to determine if balancing the variant data would improve the performance of the models. During the first round, the quality features in the training dataset were scaled to see if model performance could be improved by normalizing the quality features and removing repetitive quality features. In so doing, more focus could be placed on the quality features that contributed the most to the associated true or false positive variant (e.g., high-impact quality features). The impact of training on high impact quality features versus all quality features was tested using the testing dataset and a decision was made for each machine learning model to use either the high impact quality features or all the quality features, based on the coefficient or importance values for each quality feature. The coefficient or importance values reveal the contribution of each quality feature to its corresponding true positive or false positive variant label. At the end of the first round, all five cross-validated machine learning models have either been trained and tested using high-impact quality features or on all quality features.

During the second round of training and testing, oversampling techniques were used to account for the imbalance in data representation (e.g., over representation of true positive calls compared to the lesser false positive calls). The methods selected to achieve balanced datasets for evaluation included SOS, which randomly duplicates data points from the minority dataset (false positive variants), and SMOTE, which generates synthetic data points according to a k-nearest neighbor analysis of minority data point clustering. Oversampling techniques were applied to the training dataset and balanced and imbalanced data were used to train the machine learning models trained on the high-impact quality features or all quality features. The testing dataset was used to determine which oversampling technique improved the performance of the models. After completion of the first and second rounds of the second training and testing phase, optimized machine learning models were output that included: (i) models trained on all quality features and imbalanced data, (ii) models trained on all quality features and balanced data, (iii) models trained on high-impact quality features and imbalanced data, and (iii) and models trained on high-impact quality features and balanced data. These models were then selected for implementation in the Sanger bypass assay platform or system.

### Assessment of Model Performance Characteristics

### Leave-One-Out Cross-Validation Quality Feature Predictions

Multiple statistical metrics (e.g., false positive capture rate and true positive flag rate) were assessed during the initial LOOCV training and testing phase using all high-confidence variants with known truth and all available quality features. Feature weights/coefficients for HET SNVs and HOM SNVs were estimated using both raw and scaled data to determine the relative contribution of each feature to the associated true positive or false positive label (Tables 3 and 4). Only those weights/coefficients for logistic regression or importance values for, random forest and gradient boosting machine learning models are shown. When the MinMaxScaler function was applied to the logistic regression (LR) data, the function scales all the features individually into a range from 0-1, or −1-1 if there are negative values, to compress inliers within a narrow range. In so doing, the scaled LR coefficients drastically decreased or increased in value compared to the raw, unscaled LR with several quality features (average read quality, probability, and read direction probability) showing sign changes (e.g., going from positive to negative and vice versa) for both HET SNVs and HOM SNVs. Thus, it was determined that using limited, high-impact quality features for LR model training was beneficial to model performance. Scaling did not have an impact on the importance values for either the random forest model or the gradient boosting model, thus these models were trained with all quality features.

TABLE 3
Feature coefficients for HET SNVs

| | | Read | | Fwd | Rev | Fwd/rev | Avg | | Read | Read dir | Ishomo | Homo | Complex |
|---|---|---|---|---|---|---|---|---|---|---|---|---|---|
| HET SNV | Freq | count | Cov | count | count | ratio | qual | Prob | pos prob | prob | poly | poly len | Region |
| LR_coefs_raw | −0.15 | −0.23 | 0.00 | 0.20 | 0.21 | −7.54 | 0.10 | 0.47 | −1.43 | −0.58 | −0.02 | 0.07 | 2.65 |
| LR_coefs_scaled | −11.44 | −1.68 | 2.18 | 15.63 | 16.59 | −1.79 | −0.40 | −0.63 | −3.16 | 0.61 | −0.15 | 0.39 | 2.51 |
| RF_importance | 0.22 | 0.01 | 0.03 | 0.04 | 0.03 | 0.00 | 0.00 | 0.00 | 0.23 | 0.05 | 0.00 | 0.00 | 0.38 |
| GradientBoosting | 0.24 | 0.03 | 0.11 | 0.07 | 0.05 | 0.01 | 0.00 | 0.00 | 0.36 | 0.02 | 0.00 | 0.00 | 0.11 |

** Abbreviations:
Freq: frequency;
Cov: coverage;
Fwd: forward;
Rev: reverse;
Avg qual: average quality;
Prob: probability;
pos prob: position probability;
dir prob: direction probability;
Ishomo poly: homopolymer or not homopolymer;
Homo poly len: homopolymer length

TABLE 4
Feature coefficients for HOM SNVs

| | | Read | | Fwd | Rev | Fwd/rev | Avg | | Read | Read dir | Ishomo | Homo | Complex |
|---|---|---|---|---|---|---|---|---|---|---|---|---|---|
| HOM SNV | Freq | count | Cov | count | count | ratio | qual | Prob | pos prob | prob | poly | poly len | Region |
| LR_coefs_raw | −0.05 | −0.29 | 0.18 | 0.07 | 0.08 | −1.06 | −0.01 | 0.11 | −0.84 | 0.09 | 0.00 | −0.18 | 1.45 |
| LR_coefs_scaled | −6.60 | 3.40 | 3.75 | 3.28 | 3.52 | 0.39 | −0.42 | 0.00 | −3.58 | 0.77 | 0.00 | −0.65 | 2.46 |
| RF_importance | 0.26 | 0.07 | 0.08 | 0.10 | 0.09 | 0.01 | 0.01 | 0.00 | 0.21 | 0.00 | 0.00 | 0.00 | 0.17 |
| GradientBoosting | 0.06 | 0.11 | 0.09 | 0.12 | 0.16 | 0.22 | 0.18 | 0.00 | 0.01 | 0.00 | 0.00 | 0.04 | 0.00 |

** Abbreviations:
Freq: frequency;
Cov: coverage;
Fwd: forward;
Rev: reverse;
Avg qual: average quality;
Prob: probability;
pos prob: position probability;
dir prob: direction probability;
Ishomo poly: homopolymer or not homopolymer;
Homo poly len: homopolymer length

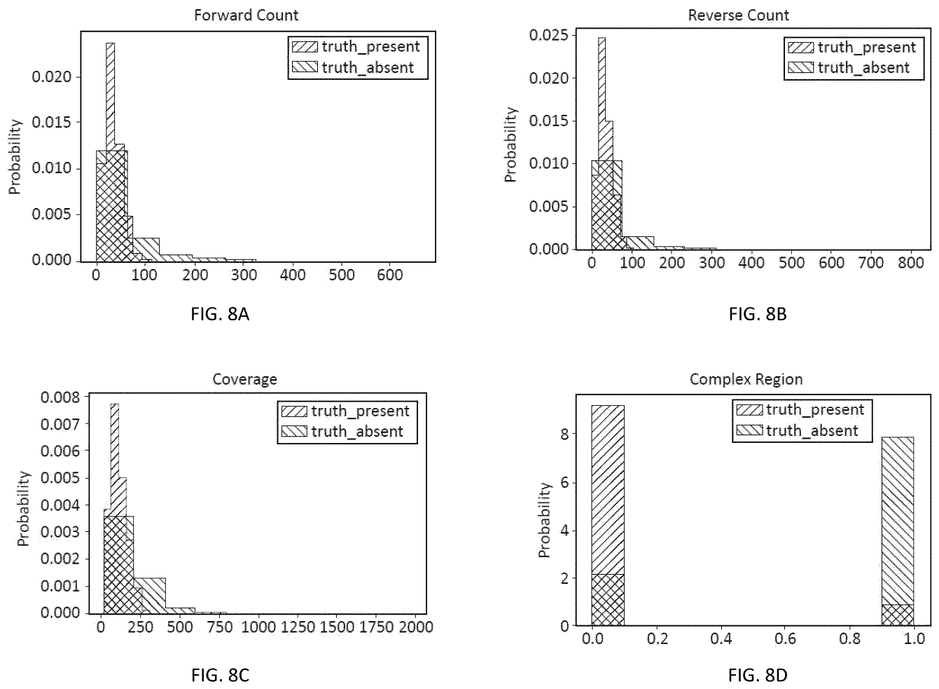

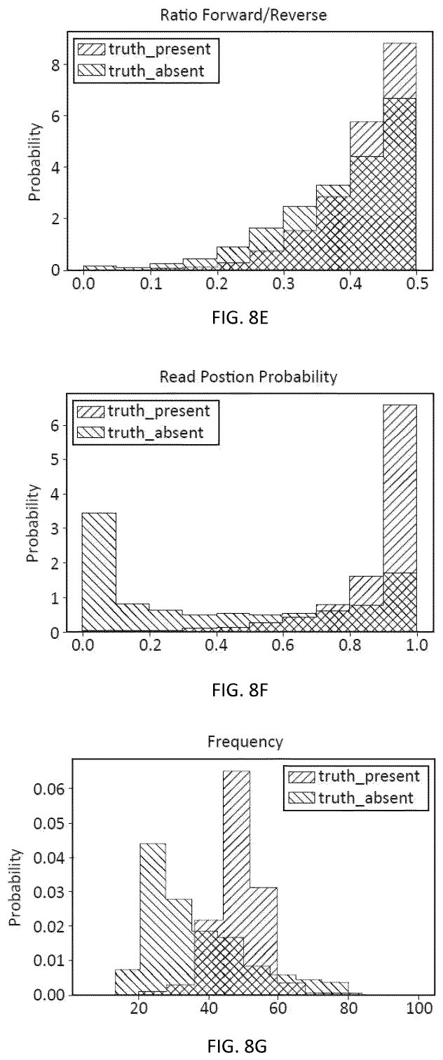

As shown in the density plots of FIGS. 8A-8G, features with positive effects (forward read count, reverse read count, coverage, and complex regions) were more likely to be associated with false positive signal (FIGS. 8A-8D). Interestingly, these features show a strong density overlap between truths that are present (purple) and truths that are absent (salmon).

On the other hand, FIGS. 8E-8G display negative features (ratio of forward/reverse reads, read position probability and read frequency) with a higher probability of being associated with true positive variants. Unlike the positive features, the negative features display better separation between truths that are present (purple) and truths that are absent (salmon). Analysis of the most optimal performing machine learning model

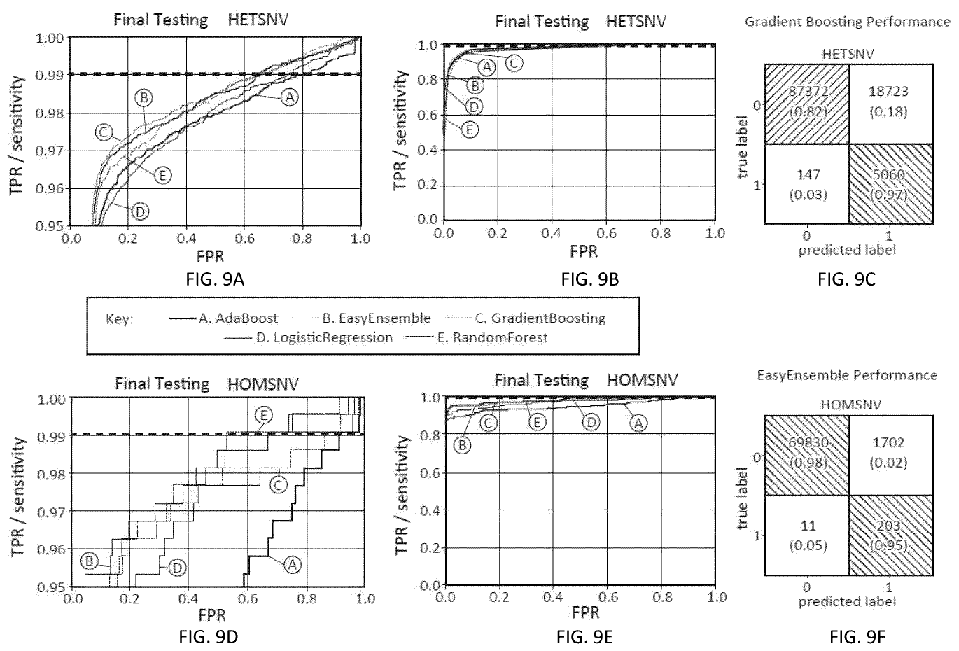

Tables 5 and 6 and FIGS. 9A-9F show summary tables and graphs respectively of the true positive and false positive rates from training various machine learning algorithms on the imbalanced raw data. Both cross-validation (Table 5) and final testing (Table 6) on all features suggested that gradient boosting is optimal for heterozygous SNV predictions, whereas EasyEnsemble performs better for homozygous SNV when considering all-around performance. However, the logistic regression and random forest models exceeded the performance of the gradient boosting and EasyEnsemble algorithms with respect to false positive capture rates. Evaluation of the cross-validation for indel variants suggested that machine-learning predictions on this class of variant are not reliable, thus indels were not tested in the final testing phase (Table 5). An alternative strategy (see FIG. 2 and Table 10 below) will be needed to determine which indels are eligible for Sanger bypass.

TABLE 5
Accuracy of calling HET SNVs, HOM SNVs, HET indels, and HOM indels predictions during cross-validation.

| | | Recall 0.95 (TPR) | | Recall 0.99 (TPR) | | |
|---|---|---|---|---|---|---|
| | | Cross-val FP | Cross-val TP | Cross-val FP | Cross-val TP | Cross-val |
| Variant/ | | capture rate | flag rate | capture rate | flag rate | ROC AUC |
| genotype | Models | (TPR %) | (FPR %) | (TPR %) | (FPR %) | (%) |
| HET | Random Forest | 94.22 +− 1.65 | 50.68 +− 8.22 | 99.07 +− 0.64 | 82.30 +− 4.86 | 92.79 +− 1.05 |
| SNVs | AdaBoost | 88.19 +− 2.88 | 12.75 +− 2.47 | 91.90 +− 2.27 | 29.62 +− 4.25 | 93.83 +− 1.04 |
| | Gradient | 91.34 +− 2.32 | 19.25 +− 3.72 | 96.56 +− 0.66 | 54.33 +− 4.26 | 94.77 +− 0.81 |
| | Boosting | | | | | |
| | EasyEnsemble | 93.81 +− 1.63 | 34.46 +− 5.17 | 98.50 +− 0.76 | 75.12 +− 5.66 | 94.34 +− 0.88 |
| | Logistic | 94.88 +− 1.52 | 41.81 +− 6.89 | 99.00 +− 0.45 | 89.43 +− 3.54 | 94.52 +− 0.71 |
| | Regression | | | | | |
| HOM | Random Forest | 87.39 +− 11.73 | 0.65 +− 0.22 | 96.91 +− 2.83 | 18.08 +− 8.96 | 98.33 +− 1.41 |
| SNVs | AdaBoost | 77.72 +− 14.24 | 0.01 +− 0.01 | 77.72 +− 14.24 | 0.01 +− 0.01 | 92.35 +− 5.93 |
| | Gradient | 80.60 +− 13.44 | 0.00 +− 0.00 | 82.16 +− 13.67 | 0.06 +− 0.05 | 96.72 +− 3.69 |
| | Boosting | | | | | |
| | EasyEnsemble | 90.82 +− 8.23 | 1.02 +− 0.35 | 93.81 +− 4.68 | 2.53 +− 1.29 | 97.87 +− 1.33 |
| | Logistic | 93.67 +− 5.97 | 19.16 +− 7.35 | 98.87 +− 1.98 | 50.52 +− 9.29 | 97.03 +− 2.23 |
| | Regression | | | | | |
| HET | Random Forest | 92.54 +− 1.92 | 87.43 +− 3.46 | 98.27 +− 1.06 | 96.91 +− 1.46 | 63.02 +− 1.94 |
| indels | AdaBoost | 71.46 +− 1.35 | 60.72 +− 2.41 | 81.56 +− 2.10 | 74.44 +− 2.62 | 58.42 +− 2.22 |
| | Gradient | 78.87 +− 2.82 | 64.68 +− 2.91 | 89.56 +− 1.75 | 80.29 +− 2.49 | 63.88 +− 1.81 |
| | Boosting | | | | | |
| | EasyEnsemble | 92.88 +− 1.55 | 85.13 +− 3.81 | 97.90 +− 1.15 | 95.07 +− 1.61 | 62.92 +− 1.95 |
| | Logistic | 94.84 +− 1.54 | 90.82 +− 3.04 | 99.02 +− 0.77 | 98.55 +− 1.01 | 62.40 +− 2.80 |
| | Regression | | | | | |
| HOM | Random Forest | 91.80 +− 2.79 | 81.74 +− 3.94 | 97.08 +− 1.68 | 93.38 +− 2.10 | 64.66 +− 2.02 |
| indels | AdaBoost | 70.64 +− 1.70 | 55.78 +− 3.04 | 78.24 +− 2.27 | 66.87 +− 3.62 | 61.97 +− 1.63 |
| | Gradient | 79.00 +− 3.44 | 58.96 +− 2.51 | 88.11 +− 2.68 | 73.41 +− 2.58 | 67.36 +− 2.14 |
| | Boosting | | | | | |
| | EasyEnsemble | 91.54 +− 2.12 | 84.45 +− 3.96 | 97.19 +− 1.59 | 93.85 +− 3.19 | 61.92 +− 1.85 |
| | Logistic | 95.20 +− 2.90 | 94.92 +− 2.39 | 98.57 +− 2.03 | 99.10 +− 1.10 | 60.02 +− 2.48 |
| | Regression | | | | | |

** Abbreviations:
TPR: true positive rate;
Cross-val: cross-validation;
FP: false positive;
FPR: false positive rate;
ROC: receiver operating characteristic;
AUC: area under the curve

TABLE 6
Model performance for heterozygous and homozygous SNVs in the
final test using imbalanced raw data and all quality features.

| | | Recall 0.99 (TPR) | | | | | |
|---|---|---|---|---|---|---|---|
| | | Final FP | Final | Final TP | | Final | |
| | | capture | TP flag | capture | Final FP | ROC | |
| Variant/ | | rate | rate | rate | flag rate | AUC | Threshold |
| genotype | Models | (TPR %) | (TPR %) | (TNR %) | (FNR %) | (%) | (%) |
| HET | Random Forest | 99.13 | 37.00 | 63.00 | 0.87 | 97.85 | 10.27 |
| SNVs | AdaBoost | 94.94 | 6.47 | 93.53 | 5.06 | 98.02 | 49.44 |
| | Gradient | 98.12 | 10.74 | 89.26 | 1.88 | 98.67 | 0.42 |
| | Boosting | | | | | | |
| | EasyEnsemble | 98.67 | 19.41 | 80.59 | 1.33 | 98.30 | 48.64 |
| | Logistic | 98.67 | 36.18 | 63.82 | 1.33 | 98.07 | 0.29 |
| | Regression | | | | | | |
| HOM | Random Forest | 98.54 | 23.72 | 76.28 | 1.46 | 99.10 | 7.12 |
| SNVs | AdaBoost | 83.68 | 0.01 | 99.99 | 16.02 | 96.34 | 51.57 |
| | Gradient | 89.32 | 0.02 | 99.98 | 10.68 | 97.90 | 1.47 |
| | Boosting | | | | | | |
| | EasyEnsemble | 95.63 | 2.67 | 97.33 | 4.37 | 98.56 | 50.17 |
| | Logistic | 99.03 | 63.98 | 36.02 | 0.97 | 97.40 | 0.02 |
| | Regression | | | | | | |

** Abbreviations:
TPR: true positive rate;
TP: true positive;
TNR: true negative rate;
FP: false positive;
FPR: false positive rate;
FNR: false negative rate;
ROC: receiver operating characteristic;
AUC: area under the curve

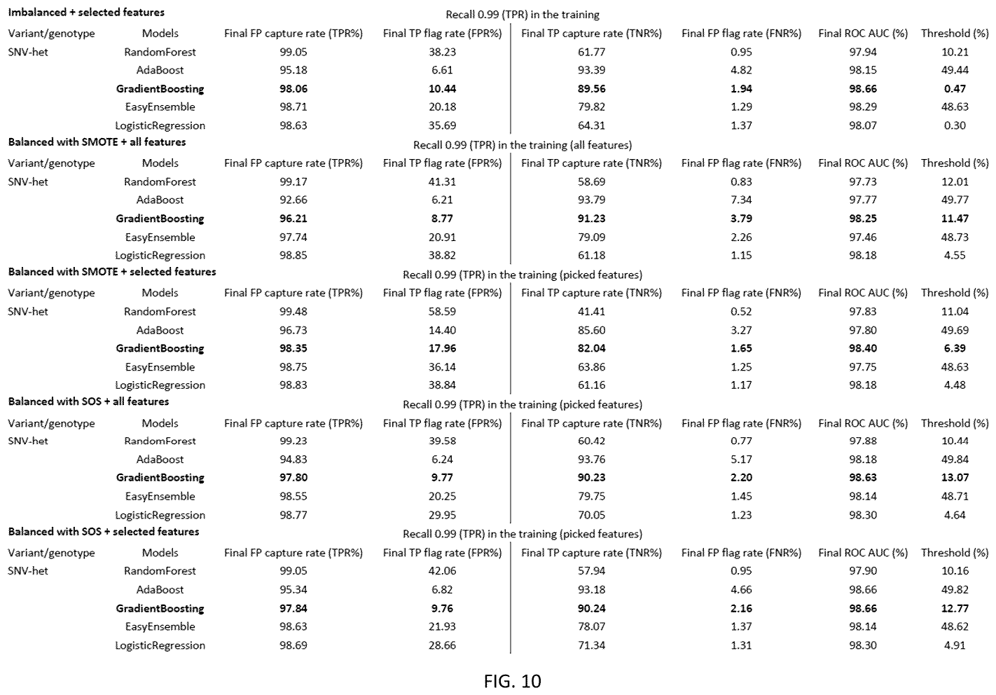

FIGS. 9A-9B display receiver operating characteristic (ROC) curves measuring the performance (e.g., the sensitivity and recall) of numerous machine learning modules including AdaBoost (W), EasyEnsemble (©), gradient boosting ((©), logistic regression (OD), and random forest ((E) in their ability to classify a HET SNV as present or absent during final testing (see FIGS. 9A and 9B). Both cross-validation testing and final testing are described in detail with respect to FIGS. 4 and 5A-5D. A ROC curve is a graphical plot that illustrates the diagnostic ability of a binary classifier system as its discrimination threshold is varied. The ROC curve is created by plotting the true positive rate (TPR) against the false positive rate (FPR) at various threshold settings. The true-positive rate is also known as sensitivity, recall or probability of detection. The false-positive rate is also known as probability of false alarm and can be calculated as (1−specificity). The ROC curve is thus the sensitivity or recall as a function of fall-out (e.g., robustness) where a model that approaches >80% is considered robust. Specifically, FIGS. 9A and 9D show zoomed-in ROC curves with sensitivity >=0.95 for the same machine learning models tested in FIGS. 9B and 9E for both HET SNV and HOM SNV predictions. During both cross-validation testing (see Table 5) and final testing (see FIG. 10 and Table 6) the EasyEnsemble machine learning model (orange) performed the best for homozygous SNV prediction.

FIG. 9C shows a confusion matrix describing the performance of the gradient boosting machine learning model to predict heterozygous SNV predictions (e.g., absent or present). A confusion matrix (also referred to as an error matrix) is a table that is used to define the performance of a classification algorithm. Each row of the matrix represents a known truth or actual value, while each column represents a predicted value. When using a 2×2 confusion matrix each of the four quadrants represents unique outcomes (e.g., starting in the top left quadrant and rotating clockwise: true negative, false positive, true positive, and false negative) that concisely summarize the results of the testing classifier, for example, the presence or absence of a variant. As illustrated in FIG. 9C. 87,372 (82%) of variants with truth labels were correctly predicted as truth by the model, 5,060 (97%) of variants labeled as false positives were successfully detected as false calls, whereas 18,723 (18%) of variants with truth labels were incorrectly predicted to be false positive and 147 (3%) false positives were missed by the model. In FIG. 9F. the confusion matrix for the performance of the EasyEnsemble machine learning model to predict HOM SNVs is shown. Specifically, 69,830 (98%) of variants with truth labels were correctly predicted as truth by the model, 203 (95%) of variants labeled as false positives were successfully detected as false calls, whereas 1,702 (2%) of variants with truth labels were incorrectly labeled as false positive and 11 (5%) false positives were missed by the model.

### Analysis of Oversampling Techniques to Generate Balanced Training Data

Table 7 and FIG. 10 show comparisons of machine-learning model performance after training with balanced vs. imbalanced datasets and all features verse select high-impact features in accordance with various embodiments. F1 scores, a machine-learning metric for precision and recall, were calculated for each combination. These data suggest that gradient boosting and random forest achieved an optimal balance between high false positive capture rates and true positive flag rates when the models were trained on the imbalanced dataset and all 13 features, whereas logistic regression performs best with SOS on limited features (frequency, read count, coverage, forward count, reverse count, forward-reverse ratio, read position probability, and complex region).

TABLE 7
Final models selection of the top performing combinations
of features and datasets for each variant type.

| | | | | | | | | Thred | | |
|---|---|---|---|---|---|---|---|---|---|---|
| Variant | Stats/Features | MLM | TPR | FPR | TNR | FNR | ROC | (%) | SS | F1 |
| HET | IMB_all | GB | 98.2 | 10.74 | 89.26 | 1.88 | 98.67 | 0.42 | 0.902 | 0.897 |
| SNV | SOS_pick | LR | 98.69 | 28.66 | 71.34 | 1.31 | 98.3 | 4.91 | 0.966 | 0.821 |
| | IMB_all | RF | 99.13 | 37 | 63 | 0.87 | 97.85 | 10.27 | 1 | 0.773 |
| HOM | SMOTE_all | RF | 98.54 | 17.52 | 82.48 | 1.46 | 99.23 | 6.12 | 0.949 | 0.883 |
| SNV | SMOTE_pick | Easy | 97.09 | 10.62 | 89.38 | 2.91 | 98.33 | 48.66 | 0.788 | 0.837 |
| | SMOTE_all | LR | 97.09 | 52.27 | 47.73 | 2.91 | 96.32 | 3.94 | 0.788 | 0.549 |

Abbreviations:
Stats: Statistics;
GB: gradient boost;
LR: logistic regression;
RF: random forest;
MLM: machine learning models;
TPR: true positive rate;
FPR: false positive rate;
TNR: true negative rate;
FNR: false negative rate;
Thred: threshold;
SS: scaled capture rate score;
IMB: imbalanced;
SOS: simple over sampling;
SMOTE: synthetic minority oversampling technique

Incorporation of the Statistical Models and Additional Filtering Criteria into a Framework for Sanger Bypass

Although gradient boosting outperformed (highest F1 score) logistic regression and random forest with respect to true positive flag rates, both models had slightly higher false positive capture rates (FIG. 10 and Table 7). In order to exploit this feature in a conservative manner, the predictions of a combined model (hereafter referred to as the “2T” model) that relied on concordant predictions at fixed probabilities was selected as the first-tier model for sanger bypass predictions (discussed in detail in FIG. 2). The 2T model comprises logistic regression and random forest models wherein the best performing model for logistic regression (SOS with select features) and random forest (raw imbalanced data, all features) were selected and the confidence thresholds were set at 99% and 90%, respectively.

Because a substantial proportion of the test variants could not be classified as a true positive or false positive by the 2T model and were thus unknown, a second tier comprising a third machine learning model was added to the Sanger bypass pipeline. In so doing, the number of unnecessary Sanger sequencing confirmations could be further limited. The chosen machine learning model was the gradient boosting model using raw imbalanced data and all features for training (described in detail in FIG. 2) due to its low true positive flag rates in performance analysis. The final logic for bypass of heterozygous SNVs combined these models with additional guardrails (e.g., variants present in complex regions are confirmed by Sanger and thresholds for allele frequency and read coverage) to capture potential false positives in regions of homology and ambiguous allele frequencies. All variants overlapping with regions of homology or low map ability would require sanger sequencing regardless of machine-learning classifications. The list of regions and genomic coordinates ineligible for Sanger bypass are maintained in a separate file used for filtering by CBI.

Table 8 shows the performance of the combined first-tier 2T model and second-tier gradient boost model on variant calls identified in the GIAB cell lines. True positive variant calls were present in both the annotated files (e.g., the whole exome sequences of the GIAB cell lines) and GIAB truth set (e.g., the high-confidence, benchmark dataset), whereas false positives are variants present in the annotated files but absent from the GIAB truth set.

TABLE 8
Performance of Sanger Bypass Model on GIAB Cell Lines

| | | | GIAB cell line variants | | |
|---|---|---|---|---|---|
| | | Gradient | True | False | |
| | 2T | Boosting | Positive | Positive | Totals |
| Models | Predicted | | 44,850 | 9 | 44,859 |
| prediction | Present | | | | |
| | Predicted | | 542 | 4,231 | 4,773 |
| | Absent | | | | |
| | Unknown | Absent | 22,119 | 5,714 | 172,857 |
| | | Present | 144,886 | 138 | |

| | | | |
|---|---|---|---|
| Total | 212,397 | 10,092 | 222,489 |

Broken down, a total of 44,859 variants were predicted to be present (true positive) by the final models where only 9 of those variants were incorrectly predicted to be present and will not be confirmed by Sanger sequencing confirmation. Moreover, the model predicted that 4,773 of the variants were absent (false positives) with only 542 incorrectly being tagged and unnecessarily receive Sanger sequencing confirmation. A total of 172,857 variants could not be classified as present or absent by the 2T model and had to be processed by the gradient boosting machine learning model in a second tier of the Sanger bypass pipeline. Gradient boosting identified 145,024 (144,886+138) variants as present and 27,833 (22,119+5,714) variants as absent; the ones tagged as ‘present’ by the gradient boosting will unnecessarily require Sanger confirmation because they are considered as true positives by the model. However, 138 of the 145,024 ‘present’ variants will incorrectly not receive Sanger sequencing confirmation due to inaccurate classification. In addition, 27,833 ‘absent’ calls will go to Sanger, even though 22,119 of them are incorrectly tagged as false positives.

In summary, 222,489 variants with known truths comprised the GIAB cell line samples. In summary, approximately 85% ((44,850+144,886)/222,489) of the total variants analyzed in the GIAB cell line samples were correctly identified as true positives and were bypassed for Sanger confirmation, approximately 4.5% ((4,231+5,714)/222,489) of the total variants were correctly identified as false positives and appropriately received Sanger sequencing confirmation, approximately 10.2% (542+22,119)/222,489) of the total variants were incorrectly identified as false negatives and would unnecessarily receive Sanger sequencing confirmation, and finally, less than 0.1% (9+138/222,489) of the total variants represent missed false positives that would incorrectly not receive Sanger sequencing confirmation. These data indicate that 80-90% of the variant calls passed through the 3 machine learning models. Further, the 2T model alone is also suggested to have a reasonably low incorrect false positive prediction rate of 0.2% (9/(9+4,231)).

Table 9 shows the performance of the combined first-tier 2T models and second-tier model on 60 reportable HET and HOM SNVs calls identified in 44 clinical samples. True positive and false positive variant calls were based on the discretion of qualified lab professionals. As observed in Table 9, 9 (2+7) out of the 60 SNVs, or 15%, were not confirmed by the model and will require confirmatory Sanger sequencing. This preliminary validation confirms the findings of the GIAB validation described in Table 8 above, further supporting the benefit of utilizing machine learning tools to reduce the number of variants that require Sanger confirmation.

TABLE 9
Performance of Sanger Bypass Model on Clinical Samples

| | Optimized model | |
|---|---|---|
| 2T_prediction | (1: fasle call, 0: true call) | Reportable SNVs |
| Absent/1 | 1 | 2 |
| | 0 | 0 |
| Present/0 | 1 | 0 |
| | 0 | 15 |
| unknown | 1 | 7 |
| | 0 | 36 |

Early assessment of machine-learning predictions on indels suggested poor performance on this category, thus an alternate strategy was needed to bypass common high-confidence variants. This strategy comprised a two-point criterion for determining which indels would be eligible for Sanger bypass. First, using the Inheritest v.2 panel, indels had to be in complete concordance between NGS and Sanger, and second, the variants also had to display allele frequency ranges and read coverage consistent with heterozygous calls. In total, five variants in high-complexity regions (Table 10) were selected for bypass. Of note, the Galt Duarte variant is eligible for sanger bypass but no longer reportable for carrier screening based on revised internal variant classification.

TABLE 10
Select heterozygous indels eligible for
Sanger Bypass on the Twist exome panel.

| | | | Inheritest |
|---|---|---|---|
| Gene | NMID | Variant | v2 catalog ID |
| GALT | NM_000155 | c.-119 -116delGTCA | GALT_293 |
| CFTR | NM_000492 | c.1521_1523delCTT | CFTR_244 |
| BBS10 | NM_024685 | c.271dupT | BBS10_5 |
| SLC37A4 | NM_001164277 | c.1042_1043delCT | SLC37A4_50 |
| HBB | NM_000518 | c.27dupG | HBB_36 |
| CFTR | NM_000492 | c.1519_1521delATC | CFTR_243 |

### Validation of the Inheritest V4 Bypass Pathway

The objective of validation in the context of the bypass logic was to determine the accuracy of predictions for variants eligible for bypass of confirmation by Sanger. Importantly, validation required assessment of the model performance on variants not previously seen during the training and testing phase of development. This validation was performed as part of the broader Inheritest v4/Twist exome panel analytical validation.

For the bypass validation component, variants identified in clinical specimens and cell lines tested across two flow cells (CBI-1289 and CBI-1810_1894) were passed to the machine-learning models for predictive classifications. Variants that did not meet reporting criteria according to the panel in which the gene overlaps (e.g., benign and likely benign variants in Inheritest genes), variants lacking GE scores (internal database of all classified variants), and variants identified in depleted specimens were excluded from the validation set. Additionally, variants with known truth were also excluded, leaving 94 variants for model assessment (Table 11). Sanger sequencing was performed to establish a truth set for each heterozygous SNV.

The concordance rate between machine-learning predictions and Sanger sequencing was 98% in this validation study (Table 11). Two variants could not be definitively confirmed by Sanger sequencing. The ERCC2 c.1847G>C variant identified in specimen 2228799078360 failed to confirm after testing with two sets of unique primers. A common SNP (chrl9:45856144G>A) identified by NGS was captured by the 2^{nd} primer design, excluding the possibility of allelic dropout, and no additional variants were observed surrounding primer-binding sites, which suggests that preferential amplification of one allele is unlikely. Notably, a minor peak consistent with the target missense change was observed in both the forward and reverse sequence when Fail Safe Buffer G was used for PCR (GC content of ERCC2 exon 20 ˜60%), but the relative imbalance in ratios at this position and the common SNP remain unexplained. Visual inspection of the raw sequence data did not provide additional insights into the cause of this discrepancy as no obvious hallmarks of a false positive variant or miscall were present (allele frequency=54.6, no apparent strand bias or position bias, no complex variants). ERCC2 exon 20 was added to the list of regions ineligible for sanger bypass. Repeat NGS to assess reproducibility may be considered at a later time. The second unconfirmed variant (MCCC2 c.1015G>A; exon 11) was identified in specimen 2228799078970. In this case, the specimen tested by NGS was depleted and an alternate tube was used for Sanger confirmation. Repeat testing of the alternate tube is required to rule out a specimen swap. MCCC2 exon 11 has also been added to list of regions ineligible for sanger bypass until the investigation into this discrepancy is resolved.

TABLE 11
Summary of sanger confirmation results for heterozygous SNVs
selected for validation of the machine-learning models.

| flow cell | sample_id | gene | nmid | cdot | chr | start |
|---|---|---|---|---|---|---|
| CBI-1289 | 2228799078470 | APC | NM_000038 | c.3920T > A | 5 | 112175211 |
| CBI-1289 | 2228799078360 | ERCC2 | NM_000400 | c.1847G > C | 19 | 45856059 |
| CBI-1289 | 2228799077420 | ERCC6 | NM_000124 | c.2167C > T | 10 | 50690735 |
| CBI-1289 | 2228799079090 | GLA | NM_000169 | c.427G > A | X | 100656740 |
| CBI-1289 | 2228799078340 | GPD1L | NM_015141 | c.839C > T | 3 | 32200588 |
| CBI-1289 | 2228799079280 | IL7R | NM_002185 | c.651G > A | 5 | 35873695 |
| CBI-1289 | 2228799078970 | MCCC2 | NM_022132 | c.1015G > A | 5 | 70936845 |
| CBI-1289 | 2228799079180 | MYH7 | NM_000257 | c.2890G > C | 14 | 23893148 |
| CBI-1289 | 2228799078340 | MYL3 | NM_000258 | c.530A > G | 3 | 46899903 |
| CBI-1289 | 2228799078890 | NGLY1 | NM_018297 | c.1201A > T | 3 | 25775422 |
| CBI-1289 | 2228799077100 | PKP2 | NM_004572 | c.1114G > A | 12 | 33021917 |
| CBI-1289 | 2228799079150 | SLC26A4 | NM_000441 | c.1151A > G | 7 | 107330570 |
| CBI-1289 | 2228799077130 | ABCB4 | NM_000443 | c.1529A > G | 7 | 87069546 |
| CBI-1289 | 2228799077340 | ACADSB | NM_001609 | c.443C > T | 10 | 124800121 |
| CBI-1289 | 2228799078360 | ALOX12B | NM_001139 | c.1562A > G | 17 | 7979005 |
| CBI-1289 | 2228799078990 | ALOXE3 | NM_021628 | c.1889C > T | 17 | 8006708 |
| CBI-1289 | 2228799078940 | BARD1 | NM_000465 | c.2137G > A | 2 | 215593597 |
| CBI-1289 | 2228799078230 | BCHE | NM_000055 | c.1253G > T | 3 | 165547569 |
| CBI-1289 | 2228799078350 | BCHE | NM_000055 | c.293A > G | 3 | 165548529 |
| CBI-1289 | 2228799078350 | BTD | NM_001370658 | c.1270G > C | 3 | 15686693 |
| CBI-1289 | 2228799079760 | BTD | NM_001370658 | c.1308A > C | 3 | 15686731 |
| CBI-1289 | 2228799077340 | BTD | NM_001370658 | c.451G > A | 3 | 15685874 |
| CBI-1289 | 2228799079150 | CLCN1 | NM_000083 | c.2680C > T | 7 | 143048771 |
| CBI-1289 | 2228799078900 | DOCK8 | NM_203447 | c.54-1G > T | 9 | 271626 |
| CBI-1289 | 2228799078460 | FBN1 | NM_000138 | c.7412C > G | 15 | 48717607 |
| CBI-1289 | 2228799079300 | FKRP | NM_024301 | c.1073C > T | 19 | 47259780 |
| CBI-1289 | 2228799078960 | G6PD | NM_000402 | c.1058T > C | X | 153761240 |
| CBI-1289 | 2228799078950 | GJB2 | NM_004004 | c.101T > C | 13 | 20763620 |
| CBI-1289 | 2228799078810 | HFE | NM_000410 | c.187C > G | 6 | 26091179 |
| CBI-1289 | 2228799078920 | HFE | NM_000410 | c.845G > A | 6 | 26093141 |
| CBI-1289 | 2228799078570 | KCNE2 | NM_172201 | c.229C > T | 21 | 35743006 |
| CBI-1289 | 2228799077500 | LDLRAP1 | NM_015627 | c.605C > A | 1 | 25889633 |
| CBI-1289 | 2228799077360 | MSH6 | NM_000179 | c.893G > A | 2 | 48026015 |
| CBI-1289 | 2228799079220 | MUTYH | NM_001128425 | c.1187G > A | 1 | 45797228 |
| CBI-1289 | 2228799079070 | MYH7 | NM_000257 | c.11C > T | 14 | 23902931 |
| CBI-1289 | 2228799078540 | NAGA | NM_000262 | c.973G > A | 22 | 42457056 |
| CBI-1289 | 2228799077340 | OCA2 | NM_000275 | c.1327G > A | 15 | 28230247 |
| CBI-1289 | 2228799077250 | PCSK9 | NM_174936 | c.1180G > A | 1 | 55523187 |
| CBI-1289 | 2228799077480 | POMGNT1 | NM_017739 | c.1539 + 1G > A | 1 | 46657769 |
| CBI-1289 | 2228799078910 | PTEN | NM_000314 | c.935A > G | 10 | 89720784 |
| CBI-1289 | 2228799079320 | SERPINA1 | NM_000295 | c.1096G > A | 14 | 94844947 |
| CBI-1289 | 2228799078070 | SOS1 | NM_005633 | c.755T > C | 2 | 39278394 |
| CBI-1289 | 2228799079040 | TCIRG1 | NM_006019 | c.1674 − 1G > A | 11 | 67816547 |
| CBI-1289 | 2228799079350 | USH2A | NM_206933 | c.6902T > C | 1 | 216144022 |
| CBI-1810_1894 | 2229399077490 | ABCB4 | NM_000443 | c.2800G > A | 7 | 87041333 |
| CBI-1810_1894 | 2229399077340 | ACADM | NM_000016 | c.799G > A | 1 | 76215194 |
| CBI-1810_1894 | 2229399079860 | ACADM | NM_000016 | c.985A > G | 1 | 76226846 |
| CBI-1810_1894 | 2228799079820 | ALDOB | NM_000035 | c.448G > C | 9 | 104189856 |
| CBI-1810_1894 | 2228799079840 | ATM | NM_000051 | c.4784A > G | 11 | 108165661 |
| CBI-1810_1894 | 2228799080240 | ATP7B | NM_000053 | c.3316G > A | 13 | 52516618 |
| CBI-1810_1894 | 2229399077440 | ATP7B | NM_000053 | c.2605G > A | 13 | 52524268 |
| CBI-1810_1894 | 2229399079920 | ATP7B | NM_000053 | c.1995G > A | 13 | 52534410 |
| CBI-1810_1894 | 2228799079810 | ATP7B | NM_000053 | c.347T > C | 13 | 52549009 |
| CBI-1810_1894 | 2228799080200 | BBS1 | NM_024649 | c.1169T > G | 11 | 66293652 |
| CBI-1810_1894 | 2229399080560 | BBS2 | NM_031885 | c.1895G > C | 16 | 56530894 |
| CBI-1810_1894 | 2229399080570 | BRCA2 | NM_000059 | c.4819A > G | 13 | 32913311 |
| CBI-1810_1894 | 2229399079860 | CAPN3 | NM_000070 | c.2257G > A | 15 | 42702858 |
| CBI-1810_1894 | 2228799080310 | CYP1B1 | NM_000104 | c.1405C > T | 2 | 38298092 |
| CBI-1810_1894 | 2229399077520 | DPYD | NM_000110 | c.2846A > T | 1 | 97547947 |
| CBI-1810_1894 | 2229399079940 | FAH | NM_000137 | c.782C > T | 15 | 80465431 |
| CBI-1810_1894 | 2229399080570 | GJB2 | NM_004004 | c.109G > A | 13 | 20763612 |
| CBI-1810_1894 | 2228799080210 | GJB2 | NM_004004 | c.35G > T | 13 | 20763686 |
| CBI-1810_1894 | 2228799080100 | GLA | NM_000169 | c.352C > T | X | 100658816 |
| CBI-1810_1894 | 2229399077700 | GLDC | NM_000170 | c.2216G > A | 9 | 6554768 |
| CBI-1810_1894 | 2229399080620 | HBB | NM_000518 | c.316 − 197C > T | 11 | 5247153 |
| CBI-1810_1894 | 2228799079860 | HLCS | NM_000411 | c.1519 + 5G > A | 21 | 38139514 |
| CBI-1810_1894 | 2229399080520 | HSD17B4 | NM_000414 | c.743G > A | 5 | 118829516 |
| CBI-1810_1894 | 2229399079780 | MEFV | NM_000243 | c.2177T > C | 16 | 3293310 |
| CBI-1810_1894 | 2229399080040 | MEFV | NM_000243 | c.2082G > A | 16 | 3293405 |
| CBI-1810_1894 | 2229399079850 | MPL | NM_005373 | c.305G > C | 1 | 43804305 |
| CBI-1810_1894 | 2229399080550 | NBN | NM_002485 | c.127C > T | 8 | 90994994 |
| CBI-1810_1894 | 2228799080370 | NDUFAF5 | NM_024120 | c.519 + 4A > G | 20 | 13779150 |
| CBI-1810_1894 | 2229399080700 | NPHS1 | NM_004646 | c.565G > T | 19 | 36341309 |
| CBI-1810_1894 | 2229399080020 | NPHS2 | NM_014625 | c.686G > A | 1 | 179526214 |
| CBI-1810_1894 | 2228799079920 | PAH | NM_000277 | c.1068C > G | 12 | 103237555 |
| CBI-1810_1894 | 2229399080430 | PAH | NM_000277 | c.688G > A | 12 | 103248932 |
| CBI-1810_1894 | 2228799079910 | PMM2 | NM_000303 | c.422G > A | 16 | 8905010 |
| CBI-1810_1894 | 2229399079940 | PMM2 | NM_000303 | c.713G > A | 16 | 8941654 |
| CBI-1810_1894 | 2228799079950 | RAG1 | NM_000448 | c.1566G > T | 11 | 36596420 |
| CBI-1810_1894 | 2229399080690 | RPGRIP1 | NM_020366 | c.3358A > G | 14 | 21811213 |
| CBI-1810_1894 | 2229399077300 | SMPD1 | NM_000543 | c.1550A > T | 11 | 6415491 |
| CBI-1810_1894 | 2229399077710 | USH2A | NM_206933 | c.12145G > A | 1 | 215853640 |
| CBI-1810_1894 | 2229399077640 | ABCC8 | NM_000352 | c.3976G > A | 11 | 17418752 |
| CBI-1810_1894 | 2229399080670 | ACSF3 | NM_174917 | c.1081G > A | 16 | 89180850 |
| CBI-1810_1894 | 2228799080180 | ASL | NM_000048 | c.571C > T | 7 | 65551777 |
| CBI-1810_1894 | 2228799079900 | CPT2 | NM_000098 | c.149C > A | 1 | 53662764 |
| CBI-1810_1894 | 2229399080690 | DHCR7 | NM_001360 | c.1342G > A | 11 | 71146507 |
| CBI-1810_1894 | 2229399077670 | FKRP | NM_024301 | c.826C > A | 19 | 47259533 |
| CBI-1810_1894 | 2229399079950 | GALK1 | NM_000154 | c.1036G > A | 17 | 73754362 |
| CBI-1810_1894 | 2229399077450 | GCDH | NM_000159 | c.262C > A | 19 | 13002779 |
| CBI-1810_1894 | 2228799080170 | LDLR | NM_000527 | c.682G > T | 19 | 11216264 |
| CBI-1810_1894 | 2228799079830 | MYBPC3 | NM_000256 | c.1468G > A | 11 | 47364285 |
| CBI-1810_1894 | 2228799080130 | MYL3 | NM_000258 | c.170C > A | 3 | 46902303 |
| CBI-1810_1894 | 2228799080300 | TSEN54 | NM_207346 | c.919G > T | 17 | 73518081 |

| | | | | | | | Sanger |
|---|---|---|---|---|---|---|---|
| | | | | | | | confirm |
| flow cell | sample_id | end | ref | alt | Exon # | Strand | (Y/N) |
| CBI-1289 | 2228799078470 | 112175211 | T | A | 16 | + | Y |
| CBI-1289 | 2228799078360 | 45856059 | C | G | 20 | − | N |
| CBI-1289 | 2228799077420 | 50690735 | G | A | 10 | − | Y |
| CBI-1289 | 2228799079090 | 100656740 | C | T | 3 | − | Y |
| CBI-1289 | 2228799078340 | 32200588 | C | T | 6 | + | Y |
| CBI-1289 | 2228799079280 | 35873695 | G | A | 5 | + | Y |
| CBI-1289 | 2228799078970 | 70936845 | G | A | 11 | + | N* |
| CBI-1289 | 2228799079180 | 23893148 | C | G | 23 | − | Y |
| CBI-1289 | 2228799078340 | 46899903 | T | C | 5 | − | Y |
| CBI-1289 | 2228799078890 | 25775422 | T | A | 8 | − | Y |
| CBI-1289 | 2228799077100 | 33021917 | C | T | 4 | − | Y |
| CBI-1289 | 2228799079150 | 107330570 | A | G | 10 | + | Y |
| CBI-1289 | 2228799077130 | 87069546 | T | C | 13 | − | Y |
| CBI-1289 | 2228799077340 | 124800121 | C | T | 4 | + | Y |
| CBI-1289 | 2228799078360 | 7979005 | T | C | 12 | − | Y |
| CBI-1289 | 2228799078990 | 8006708 | G | A | 15 | − | Y |
| CBI-1289 | 2228799078940 | 215593597 | C | T | 11 | − | Y |
| CBI-1289 | 2228799078230 | 165547569 | C | A | 2 | − | Y |
| CBI-1289 | 2228799078350 | 165548529 | T | C | 2 | − | Y |
| CBI-1289 | 2228799078350 | 15686693 | G | C | 4 | + | Y |
| CBI-1289 | 2228799079760 | 15686731 | A | C | 4 | + | Y |
| CBI-1289 | 2228799077340 | 15685874 | G | A | 4 | + | Y |
| CBI-1289 | 2228799079150 | 143048771 | C | T | 23 | + | Y |
| CBI-1289 | 2228799078900 | 271626 | G | T | 2 | + | Y |
| CBI-1289 | 2228799078460 | 48717607 | G | C | 60 | − | Y |
| CBI-1289 | 2228799079300 | 47259780 | C | T | 4 | + | Y |
| CBI-1289 | 2228799078960 | 153761240 | A | G | 9 | − | Y |
| CBI-1289 | 2228799078950 | 20763620 | A | G | 2 | − | Y |
| CBI-1289 | 2228799078810 | 26091179 | C | G | 2 | + | Y |
| CBI-1289 | 2228799078920 | 26093141 | G | A | 4 | + | Y |
| CBI-1289 | 2228799078570 | 35743006 | C | T | 2 | + | Y |
| CBI-1289 | 2228799077500 | 25889633 | C | A | 6 | + | Y |
| CBI-1289 | 2228799077360 | 48026015 | G | A | 4 | + | Y |
| CBI-1289 | 2228799079220 | 45797228 | C | T | 13 | − | Y |
| CBI-1289 | 2228799079070 | 23902931 | G | A | 3 | − | Y |
| CBI-1289 | 2228799078540 | 42457056 | C | T | 8 | − | Y |
| CBI-1289 | 2228799077340 | 28230247 | C | T | 13 | − | Y |
| CBI-1289 | 2228799077250 | 55523187 | G | A | 7 | + | Y |
| CBI-1289 | 2228799077480 | 46657769 | C | T | 17 | − | Y |
| CBI-1289 | 2228799078910 | 89720784 | A | G | 8 | + | Y |
| CBI-1289 | 2228799079320 | 94844947 | C | T | 5 | − | Y |
| CBI-1289 | 2228799078070 | 39278394 | A | G | 6 | − | Y |
| CBI-1289 | 2228799079040 | 67816547 | G | A | 15 | + | Y |
| CBI-1289 | 2228799079350 | 216144022 | A | G | 36 | − | Y |
| CBI-1810_1894 | 2229399077490 | 87041333 | C | T | 23 | 1 | Y |
| CBI-1810_1894 | 2229399077340 | 76215194 | G | A | 9 | −3 | Y |
| CBI-1810_1894 | 2229399079860 | 76226846 | A | G | 11 | −3 | Y |
| CBI-1810_1894 | 2228799079820 | 104189856 | C | G | 5 | −3 | Y |
| CBI-1810_1894 | 2228799079840 | 108165661 | A | G | 32 | 0 | Y |
| CBI-1810_1894 | 2228799080240 | 52516618 | C | T | 15 | −2 | Y |
| CBI-1810_1894 | 2229399077440 | 52524268 | C | T | 11 | −3 | Y |
| CBI-1810_1894 | 2229399079920 | 52534410 | C | T | 7 | 0 | Y |
| CBI-1810_1894 | 2228799079810 | 52549009 | A | G | 2 | 0 | Y |
| CBI-1810_1894 | 2228799080200 | 66293652 | T | G | 12 | −3 | Y |
| CBI-1810_1894 | 2229399080560 | 56530894 | C | G | 15 | −3 | Y |
| CBI-1810_1894 | 2229399080570 | 32913311 | A | G | 11 | 0 | Y |
| CBI-1810_1894 | 2229399079860 | 42702858 | G | A | 21 | 0 | Y |
| CBI-1810_1894 | 2228799080310 | 38298092 | G | A | 3 | −3 | Y |
| CBI-1810_1894 | 2229399077520 | 97547947 | T | A | 22 | −3 | Y |
| CBI-1810_1894 | 2229399079940 | 80465431 | C | T | 9 | −3 | Y |
| CBI-1810_1894 | 2229399080570 | 20763612 | C | T | 2 | −3 | Y |
| CBI-1810_1894 | 2228799080210 | 20763686 | C | A | 2 | −3 | Y |
| CBI-1810_1894 | 2228799080100 | 100658816 | G | A | 2 | 0 | Y |
| CBI-1810_1894 | 2229399077700 | 6554768 | C | T | 19 | −3 | Y |
| CBI-1810_1894 | 2229399080620 | 5247153 | G | A | i2 | −3 | Y |
| CBI-1810_1894 | 2228799079860 | 38139514 | C | T | 8 | −3 | Y |
| CBI-1810_1894 | 2229399080520 | 118829516 | G | A | 11 | 0 | Y |
| CBI-1810_1894 | 2229399079780 | 3293310 | A | G | 10 | −3 | Y |
| CBI-1810_1894 | 2229399080040 | 3293405 | C | T | 10 | −3 | Y |
| CBI-1810_1894 | 2229399079850 | 43804305 | G | C | 3 | −3 | Y |
| CBI-1810_1894 | 2229399080550 | 90994994 | G | A | 2 | −3 | Y |
| CBI-1810_1894 | 2228799080370 | 13779150 | A | G | 6 | 1 | Y |
| CBI-1810_1894 | 2229399080700 | 36341309 | C | A | 5 | −3 | Y |
| CBI-1810_1894 | 2229399080020 | 179526214 | C | T | 5 | −1 | Y |
| CBI-1810_1894 | 2228799079920 | 103237555 | G | C | 11 | −3 | Y |
| CBI-1810_1894 | 2229399080430 | 103248932 | C | T | 6 | −3 | Y |
| CBI-1810_1894 | 2228799079910 | 8905010 | G | A | 5 | −3 | Y |
| CBI-1810_1894 | 2229399079940 | 8941654 | G | A | 8 | 0 | Y |
| CBI-1810_1894 | 2228799079950 | 36596420 | G | T | 2 | −3 | Y |
| CBI-1810_1894 | 2229399080690 | 21811213 | A | G | 22 | 0 | Y |
| CBI-1810_1894 | 2229399077300 | 6415491 | A | T | 6 | 1 | Y |
| CBI-1810_1894 | 2229399077710 | 215853640 | C | T | 62 | −1 | Y |
| CBI-1810_1894 | 2229399077640 | 17418752 | C | T | 32 | 0 | Y |
| CBI-1810_1894 | 2229399080670 | 89180850 | G | A | 6 | 0 | Y |
| CBI-1810_1894 | 2228799080180 | 65551777 | C | T | 8 | −3 | Y |
| CBI-1810_1894 | 2228799079900 | 53662764 | C | A | 1 | −3 | Y |
| CBI-1810_1894 | 2229399080690 | 71146507 | C | T | 9 | −3 | Y |
| CBI-1810_1894 | 2229399077670 | 47259533 | C | A | 4 | −3 | Y |
| CBI-1810_1894 | 2229399079950 | 73754362 | C | T | 7 | −2 | Y |
| CBI-1810_1894 | 2229399077450 | 13002779 | C | A | 4 | −2 | Y |
| CBI-1810_1894 | 2228799080170 | 11216264 | G | T | 4 | −3 | Y |
| CBI-1810_1894 | 2228799079830 | 47364285 | C | T | 16 | 0 | Y |
| CBI-1810_1894 | 2228799080130 | 46902303 | G | T | 3 | 0 | Y |
| CBI-1810_1894 | 2228799080300 | 73518081 | G | T | 8 | −3 | Y |

### VII. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

1.-28. (canceled)

29. A computer-implemented method comprising:
performing next generation sequencing (NGS) on nucleic acid obtained from a biological sample of a subject to generate sequencing data;
extracting information of a set of variants from the sequencing data, wherein the information of the set of variants comprises a type of each variant in the set of variants and one or more quality features of each variant in the set of variants;
clustering the set of variants into one or more subsets of variants based on the type of each variant in the set of variants;
generating, using a first machine learning model, a predicted status of each variant in at least one subset of the one or more subsets of variants based on the one or more quality features, wherein the predicted status is a presence status, an absence status, or an unknown status;
generating, using a second machine learning model, a confirmatory status of each variant with the unknown status as the predicted status, wherein the confirmatory status is a presence status or an absence status; and
performing Sanger sequencing on nucleic acid molecules comprising variants with the absence status as the predicted status or the confirmatory status to confirm an existence of the variants.

30. The computer-implemented method of claim 29, further comprising generating a testing report for the subject based on the sequencing data, the information of the set of variants, the predicted status of each variant in the at least one subset of the one or more subsets of variants, the confirmatory status of each variant with the unknown status as the predicted status, and/or results of the Sanger sequencing.

31. The computer-implemented method of claim 29, wherein the type of each variant is a heterozygous single nucleotide variant (SNV), a homozygous SNV, a heterozygous insertion-deletion (indel), or a homozygous indel.

32. (canceled)

33. The computer-implemented method of claim 29, further comprising performing Sanger sequencing on regions corresponding to homozygous SNVs or homozygous indels.

34. (canceled)

35. The computer-implemented method of claim 29, further comprising:
extracting information of a second set of variants from the sequencing data, wherein the second set of variants comprises variants in complexity regions; and
performing Sanger sequencing on regions corresponding to the second set of variants.

36. The computer-implemented method of claim 29, further comprising:
determining (i) an allele frequency and (ii) a read coverage for a variant with a present or unknown status as the predicted status;
determining (i) the allele frequency or (ii) the read coverage failing a predetermined criterion; and
performing Sanger sequencing on a region corresponding to the variant.

37-38. (canceled)

39. The computer-implemented method of claim 29, further comprising:
performing NGS on reference samples obtained from a database to generate reference sequencing data; and
training the first machine learning model and the second machine learning model using labeled variant data obtained from the database and the reference sequencing data.

40-42. (canceled)

43. A system comprising:
one or more data processors; and
a non-transitory computer readable medium storing instructions which, when executed on the one or more data processors, cause the one or more data processors to perform operations comprising:
performing next generation sequencing (NGS) on nucleic acid obtained from a biological sample of a subject to generate sequencing data;
extracting information of a set of variants from the sequencing data, wherein the information of the set of variants comprises a type of each variant in the set of variants and one or more quality features of each variant in the set of variants;
clustering the set of variants into one or more subsets of variants based on the type of each variant in the set of variants;
generating, using a first machine learning model, a predicted status of each variant in at least one subset of the one or more subsets of variants based on the one or more quality features, wherein the predicted status is a presence status, an absence status, or an unknown status;
generating, using a second machine learning model, a confirmatory status of each variant with the unknown status as the predicted status, wherein the confirmatory status is a presence status or an absence status; and
performing Sanger sequencing on nucleic acid molecules comprising variants with the absence status as the predicted status or the confirmatory status to confirm an existence of the variants.

44. The system of claim 43, wherein the operations further comprise generating a testing report for the subject based on the sequencing data, the information of the set of variants, the predicted status of each variant in the at least one subset of the one or more subsets of variants, the confirmatory status of each variant with the unknown status as the predicted status, and/or results of the Sanger sequencing.

45. The system of claim 43, wherein the type of each variant is a heterozygous single nucleotide variant (SNV), a homozygous SNV, a heterozygous insertion-deletion (indel), or a homozygous indel.

46. (canceled)

47. The system of claim 43, wherein the operations further comprise performing Sanger sequencing on regions corresponding to homozygous SNVs or homozygous indels.

48. (canceled)

49. The system of claim 43, wherein the operations further comprise:
extracting information of a second set of variants from the sequencing data, wherein the second set of variants comprises variants in complexity regions; and
performing Sanger sequencing on regions corresponding to the second set of variants.

50. The system of claim 43, wherein the operations further comprise:
determining (i) an allele frequency and (ii) a read coverage for a variant with a present or unknown status as the predicted status;
determining (i) the allele frequency or (ii) the read coverage failing a predetermined criterion; and
performing Sanger sequencing on a region corresponding to the variant.

51-52. (canceled)

53. The system of claim 43, wherein the operations further comprise:
performing NGS on reference samples obtained from a database to generate reference sequencing data; and
training the first machine learning model and the second machine learning model using labeled variant data obtained from the database and the reference sequencing data.

54. (canceled)

55. A computer-program product tangibly embodied in a non-transitory machine-readable medium, including instructions configured to cause one or more data processors to perform operations comprising:
performing next generation sequencing (NGS) on nucleic acid obtained from a biological sample of a subject to generate sequencing data;
extracting information of a set of variants from the sequencing data, wherein the information of the set of variants comprises a type of each variant in the set of variants and one or more quality features of each variant in the set of variants;
clustering the set of variants into one or more subsets of variants based on the type of each variant in the set of variants;
generating, using a first machine learning model, a predicted status of each variant in at least one subset of the one or more subsets of variants based on the one or more quality features, wherein the predicted status is a presence status, an absence status, or an unknown status;
generating, using a second machine learning model, a confirmatory status of each variant with the unknown status as the predicted status, wherein the confirmatory status is a presence status or an absence status; and
performing Sanger sequencing on nucleic acid molecules comprising variants with the absence status as the predicted status or the confirmatory status to confirm an existence of the variants.

56. The computer-program product of claim 55, wherein the operations further comprise generating a testing report for the subject based on the sequencing data, the information of the set of variants, the predicted status of each variant in the at least one subset of the one or more subsets of variants, the confirmatory status of each variant with the unknown status as the predicted status, and/or results of the Sanger sequencing.

57. The computer-program product of claim 55, wherein the type of each variant is a heterozygous single nucleotide variant (SNV), a homozygous SNV, a heterozygous insertion-deletion (indel), or a homozygous indel.

58. (canceled)

59. The computer-program product of claim 55, wherein the operations further comprise performing Sanger sequencing on regions corresponding to homozygous SNVs or homozygous indels.

60. (canceled)

61. The computer-program product of claim 55, wherein the operations further comprise:
extracting information of a second set of variants from the sequencing data, wherein the second set of variants comprises variants in complexity regions; and
performing Sanger sequencing on regions corresponding to the second set of variants.

62. The computer-program product of claim 55, wherein the operations further comprise:
determining (i) an allele frequency and (ii) a read coverage for a variant with a present or unknown status as the predicted status;
determining (i) the allele frequency or (ii) the read coverage failing a predetermined criterion; and
performing Sanger sequencing on a region corresponding to the variant.

63-66. (canceled)
